SEQUENCE LISTING (16 sequences)

SEQ ID NO: 1 - protein, 338 aa, Lycopersicon esculentum
MGRTPCCEKVGIKRGRWTAEEDQILTNYIISNGEGSWRSLPKNAGLLRCGKSCRLRWINYLRSDLKRGNITSQEEDIIIKLHATLGNRWSLIAEHLSGRTDNEIKNYWNSHLSRKVDSLRIPSDEKLPKAVVDLAKKGIPKPIKKSSISRPKNKKSNLLEKEALCCTNMPACDSAMELMQEDLAKIEVPNSWAGPIEAKGSLSSDSDIEWPRLEEIMPDVVIDDEDKNTNFILNCFREEVTSNNVGNSYSCIEEGNKKISSDDEKIKLLMDWQDNDELVWPTLPWELETDIVPSWPQWDDTDTNLLQNCTNDNNNYEEATTMEINNQNHSTIVSWLLS

SEQ ID NO: 2 - protein, 60 aa, Lycopersicon esculentum
MGRTPCCEKVGIKRGRWTAEEDQILTNYIISNGEGSWRSLPKNAGLLRCGKSCRLRWINY

SEQ ID NO: 3 - protein, 338 aa, Lycopersicon esculentum
MGRTPCCEKVGIKRGRWTAEEDQILTNYIISNGEGSWRSLPKNAGLLRCRKSCRLRWINYLRSDLKRGNITSQEEDIIIKLHATLGNRWSLIAEHLSGRTDNEIKNYWNSHLSRKVDSLRIPSDEKLPKAVVDLAKKGIPKPIKKSSISRPKNKKSNLLEKEALCCTNMPACDSAMELMQEDLAKIEVPNSWAGPIEAKGSLSSDSDIEWPRLEEIMPDVVIDDEDKNTNFILNCFREEVTSNNVGNSYSCIEEGNKKISSDDEKIKLLMDWQDNDELVWPTLPWELETDIVPSWPQWDDTDTNLLQNCTNDNNNYEEATTMEINNQNHSTIVSWLLS

SEQ ID NO: 4 - DNA, 1017 bp, Lycopersicon esculentum
atgggaagaacaccttgttgtgaaaaagtgggcatcaagagaggcagatggactgcagaagaagatcaaattctcactaattatattatttctaatggagaaggctcttggaggtcgttacctaaaaatgccggattattgagatgcggaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaattaataaccaaaatcatagtaccattgtatcttggcttttgtcttag

SEQ ID NO: 5 - DNA, 1017 bp, Lycopersicon esculentum
atgggaagaacaccttgttgtgaaaaagtgggcatcaagagaggcagatggactgcagaagaagatcaaattctcactaattatattatttctaatggagaaggctcttggaggtcgttacctaaaaatgccggattattgagatgcggaaagagttgtagactacgatggattaattattagaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaattaataaccaaaatcatagtaccattgtatcttggcttttgtcttag

SEQ ID NO: 6 - DNA, 1017 bp, Lycopersicon esculentum
atgggaagaacaccttgttgtgaaaaagtgggcatcaagagaggcagatggactgcagaagaagatcaaattctcactaattatattatttctaatggagaaggctcttggaggtcgttacctaaaaatgccggattattgagatgccgaaagagttgtagactacgatggattaattatttgaggtctgatctcaagagagggaacattacttctcaagaggaagatataattataaagttacatgcaactttgggtaacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaattaataaccaaaatcatagtaccattgtatcttggcttttgtcttag

SEQ ID NO: 7 - DNA, 5185 bp, Lycopersicon esculentum
  exon (1034)..(1167): Exon 1
  exon (1258)..(1386): Exon 2
  exon (2824)..(3173): Exon 3
  exon (3767)..(4170): Exon 4
gatgttgatcgaaagggagcgaatagagtacgttatttgatttttaaataataatactcgaggtttgaaaataaaattttagaacaatcgaaattgagggtatcgaatatgtttaaccttaaaacggtacaattatttagatttatagacttcaatcaaggacatatataaggattaagatagtcacaaacacgtgatttgtatactatattttgattaatgaatgggcaaattaaaatgaaataacaaatatatatttataacgagacataaatacgttaagtaaatagaattagttctgaaatccgatgtcgatagctagcaacgtcatcgaataaccttgattatattggtattaattgaaacaaatataacgtagaacaaattaagctcaagatcttaaaaacacataaagatattacattactacaaatgaattaaaaaatgcgataatttacaatgaaggaaaaggaatttttttattaagtaaaatcatagagtaatcaccacccactatgactcccatctacctggttaaagaaaaattagcataaaaaagtcttttatatatatatatatatatatgaagcaaagtgttctaattatgaataaagaaatatttattagattataacgatgattatatttaggatggagctagcaatttatcagaggattcacctcttttaatgaaaaatattattatctgtacataattaaaatgatttttttttataatatacaataaatatcaaattcccttcaattattttatacatttatttttttaagttttaaatttttttttattaaaaatcttaactcttcttttatcaaagagtgacatgcaatgcaaaaaagcttattaagtcaacctttggtacgttattaagttcacataaaattaactagactaaagtgaagagcggggtccatttatttgtgttgtctctctatttattggcatttctattggtgaaatgagactaattttcattgccttttgcttctccattttgtgataataataataatgggaagaacaccttgttgtgaaaaagtgggcatcaagagaggcagatggactgcagaagaagatcaaattctcactaattatattatttctaatggagaaggctcttggaggtcgttacctaaaaatgccggtacgattacctactaatcttttattttaatttgaaatttaaaatttttttcttcgtttaacagtttttttataatattttatttcgaaggattattgagatgcggaaagagttgtagactacgatggattaattatttgaggtctgatctcaagagagggaacattacttctcaagaggaagatataattataaagttacatgcaactttgggtaacaggtaattagtcaattacttgattggactttttagcttgctaattaaaccactcattttgtttctttttagtctaggtccagaaaaaaatgtctcctttaaaatcaagtactttcttcgtttaaaaaataataattttatttttatttagtctgttttataaagaatgactttttttttagtaatatgttaaatttaatttttcacatgacatctttaaaattataaaattagagatagtttgatacatttgacataactttaatttagaatcacttctttcttttcttaaaatccgtttcaagtcaaataggtcattcttttttatacgcaagaagtatttttttctttaaaaataaatctgaaactcattttaggttataaacattgtcacaataatttggtgcccgatctaacaacacttcttatatcattttagtgtgtgaatagtgttacaccaaatttaatacaacaaaattactcatcaaaattattactattcatgataacatagtgtaatggattcgagctagagaaagaataaataatatgttttaggtaaataatattaatggattcgagctagagaaagaataaataatatgttttaggtaaataatattccatttgcttaaaaaaataatctttttttttaaaaaaagaatgatttcttttactttcagatatattttaatctcagctgttgttcgtgtgataagtttaatatcatataatactttgctttatttgacataattttaatttagatttataaaattaataattttttttattttcttaaatatcgtgttaaattaaactaggtcaatggtataattgattgaagtagatgccctaataaataaaagtgagatcaatgcaattataattaacttaaattcatcacttcttttttactacttgaattcatcacataaaacaaatgaatttttcttcttcttttatttcatgtttactccagtacttaatagtttatagttatgtttgcctggaaaaaggagaaaagttttggtcactttaatttgtagggtattattttctacattcattatttgtgctaatgaattaataagttaattaaattggtcccctcgagtaagttcaatattactcttttttttttctttttcatatgacgagtgacatattcatgctttaaaaacaattcatcctttctattattagtcatataccaagtctagaaaataaaacagtgacaatttaaagtattttttcaaactagaaaacgtatcttaagttggatgtatacacaaatatatcaaataatttcaacaaagaaaaaatttagaaaagatgtgttagttgtgagttgtgacattaaatatgattgattaatacaatataccatcgatctagtttctaacattttctagtatcatcgactttttaaaattacagatggtctcttatagcagaacatttatcaggtagaacagacaatgagataaaaaactattggaactctcatctaagtcgaaaagttgatagcttaaggataccaagcgatgagaagttacctaaagccgtagttgatttggctaaaaaaggtataccgaagccaattaaaaaatcatcgattagtcgaccaaaaaataaaaagtcaaacttattagaaaaagaagcattgtgttgtacaaatatgccagcttgtgatagtgccatggaattaatgcaagaagatctagcaaagatagaggtgccaaattcttgggcaggacctatagaggccaagggaagccttagttcaggtacaaatttcgatgttttgactattttttattgtgaaatttgattttaaaaaatattttttgatattaaagtgaaaaataatattcaaaatttatttaagttgtgtttggttatgaatatgaattagagttgtttttttcattttttcctcaattatttcgagtaaacctttttttttctttaaagaattgaaattttattgtcaaatatgattctctgagttttaactatcgaaaaaagcgaaaaagatcaaacaccctctaatagtttttattaatcaattaaatacattttcaatagtgactatgacgacataatatttatatattgaaatatatgattattttatcaaaaaacttaaaatttaattttcacgtctttcttttcttttgaaacgtcattttttatatgtaccttttagatccaatatctatctatggatagacgttgcgaagtactttttgttattttcaattattaggcacaaataattgaatctagcacctcttgtatgtacaaaattttaaactgtagcaataaataaatatattttttaatttttttaaatttttatttttttttgtctgagcagatagtgatatcgaatggccaagactcgaggagattatgccagacgtggtgattgatgatgaagataagaacacaaatttcatattgaattgtttcagagaagaagtaacgagcaataatgtagggaatagttattcatgtatcgaggaaggtaataaaaagatatcaagcgacgatgaaaaaatcaaattattaatggattggcaagataatgatgagttagtatggccaacgttaccatgggaattagaaacggatatagttcccagttggccacaatgggacgatactgacactaacttacttcaaaattgcaccaatgataataataattatgaagaagcaacaacaatggaaattaataaccaaaatcatagtaccattgtatcttggcttttgtcttagaaatataataatatgacattatatattgcttttgaatatattactcaactctttttgtttcgttttatatttggaatgtgggaattagaatgactagtttatgtacatattttaagtttcgttagaaatatcgtcaagtcagattaaaatatgtatgagttgatgtagtaataaatgttattgttattactttttttgatgtaattgaagtgtcttaatttgattagaaagtaaagttaaaatacagtgaaattttttaaaaatttaatttttttttgtttagaatatcaaattaatataatctaatttaatttttaaaattaattaaattgactttcgaaaatggaacatgaaaattaaaagtggagaagtagaaaaacactgacaaattaattaaacaaagaagaataaacaaatatgctcataaacttggttttatttagtatttatgatctttcaattacatataagtagatatttaaatttgtataaagttaaacaaataaatatcatgtgacatattacacgtaaaatatcatgtaaacgtaaattgttatgcacaacatgtatatttagttttttaattccttacaaaagaaaacatttaaagaaaaaaactaatcattaaaaatagacactttattaaattgaacgaaataaaatactaaattgagaaaaataataattaggcttttataatacattgataaaacaaaccagatataacacatgaatatgctcataaaagtagtctcatttaatatatatatatatatatatatactaaaaatttatataaaattatacaaataaatatatgtcttacataatatttcccgagaatcaataagaaaataagtgctaagtttcagatcagtaacgaactggcttcttgagaagctcattcccaccgcacagctccagcttgcaaccacaggggcccgtcaattatcaaataaaaaagggcaacttatttattctacttattcaatttttaataaatctaagtaaccat

SEQ ID NO: 8 - protein, 338 aa, Lycopersicon esculentum
MGRTPCCEKVGIKRGRWTAEEDQILTNYIISNGEGSWRSLPKNAGLLRCGKSCRLRWINYLRSDLKRRNITSQEEDIIIKLHATLGNRWSLIAEHLSGRTDNEIKNYWNSHLSRKVDSLRIPSDEKLPKAVVDLAKKGIPKPIKKSSISRPKNKKSNLLEKEALCCTNMPACDSAMELMQEDLAKIEVPNSWAGPIEAKGSLSSDSDIEWPRLEEIMPDVVIDDEDKNTNFILNCFREEVTSNNVGNSYSCIEEGNKKISSDDEKIKLLMDWQDNDELVWPTLPWELETDIVPSWPQWDDTDTNLLQNCTNDNNNYEEATTMEINNQNHSTIVSWLLS

SEQ ID NO: 9 - protein, 338 aa, Lycopersicon esculentum
MGRTPCCEKVGIKRGRWTAKEDQILTNYIISNGEGSWRSLPKNAGLLRCGKSCRLRWINYLRSDLKRGNITSQEEDIIIKLHATLGNRWSLIAEHLSGRTDNEIKNYWNSHLSRKVDSLRIPSDEKLPKAVVDLAKKGIPKPIKKSSISRPKNKKSNLLEKEALCCTNMPACDSAMELMQEDLAKIEVPNSWAGPIEAKGSLSSDSDIEWPRLEEIMPDVVIDDEDKNTNFILNCFREEVTSNNVGNSYSCIEEGNKKISSDDEKIKLLMDWQDNDELVWPTLPWELETDIVPSWPQWDDTDTNLLQNCTNDNNNYEEATTMEINNQNHSTIVSWLLS

SEQ ID NO: 10 - protein, 821 aa, Lycopersicon esculentum
MNFGGFLDNNSGGGGARIVADIPFNHNNSSSNNDNKNNMPTGAISQPRLLPQSLAKNMFNSPGLSLALQTGMEGQSEVTRMAENYEGNNSVGRRSREEEPDSRSGSDNLEGASGDEQDATDKPPRKKRYHRHTPQQIQELESLFKECPHPDEKQRLELSKRLSLETRQVKFWFQNRRTQMKTQLERHENSILRQENDKLRAENMSIREAMRNPICTNCGGPAMIGEISLEEQHLRIENARLKDELDRVCALAGKFLGRPISSLVTSMPPPMPNSSLELGVGSNGFGGMSNVPTTLPLAPPDFGVGISNSLPVVPSTRQSTGIERSLERSMYLELALAAMEELVKMAQTDEPLWFRSIEGGREILNHEEYIRTFTPCIGMRPNSFISEASRETGMVIINSLALVETLMDSNKWAEMFPCLIARTSTTDVISSGMGGTRNGALQLMHAELQVLSPLVPIREVNFLRFCKQHAEGVWAVVDVSIDTIRETSGAPTFPNSRRLPSGCVVQDMPNGYSKVTWVEHAEYEEGANHHLYRQLISAGMGFGAQRWVATLQRQCECLAILMSSTVSARDHTAITPSGRRSMLKLAQRMTNNFCAGVCASTVHKWNKLCAGNVDEDVRVMTRKSVDDPGEPAGIVLSAATSVWLPVSPQRLFDFLRDERLRSEWDILSNGGPMQEMAHIAKGQDHGNCVSLLRASAMNANQSSMLILQETCIDAAGALVVYAPVDIPAMHVVMNGGDSAYVALLPSGFSIVPDGPGSRGSNGPSCNGGPDQRISGSLLTVAFQILVNSLPTAKLTVESVETVNNLISCTVQKIKAALQCES

SEQ ID NO: 11 - protein, 821 aa, Lycopersicon esculentum
MNFGGFLDNNSGGGGARIVADIPFNHNNSSSNNDNKNNMPTGAISQPRLLPQSLAKNMFNSPGLSLALQTGMEGQSEVTRMAENYEGNNSVGRRSREEEPDSRSGSDNLEGASGDEQDATDKPPRKKRYHRHTPQQIQELESLFKECPHPDEKQRLELSKRLSLETRQVKFWFQNRRTQMKTQLERHENSILRQENDKLRAENMSIREAMRNPICTNCGGPAMIGEISLEEQHLRIENARLKDELDRVCALAGKFLGRPISSLVTSMPPPMPNSSLELGVGSNGFGGMSNVPTTLPLAPPDFGVGISNSLPVVPSTRQSTGIERSLERSMYLELALAAMEELVKMAQTDEPLWFRSIEGGREILNHEEYIRTFTPCIGMRPNSFISEASRETGMVIINSLALVETLMDSNKWAEMFPCLIARTSTTDVISSGMGGTRNGALQLMHAELQVLSPLVPIREVNFLRFCKQHAEGVWAVVDVSIDTIRETSGAPTFPNSRRLPSGCVVQDMPNGYSKVTWVEHAEYEEGANHHLYRQLISAGMGFGAQRWVATLQRQCECLAILMSSTVSARDHTAITPSGRRSMLKLAQRMTNNFCAGVCASTVHKWNKLCAGNVDEDVRVMTRKSVDDPGEPAGIVLSAATSVWLPVSPQRLFDFLRDERLRSEWDILSNGGPMQEMAHIAKGQDHGNCVSLLRASAMNANQSSMLILQETCIDAAGALVVYAPVDIPAMHVVMNGVDSAYVALLPSGFSIVPDGPGSRGSNGPSCNGGPDQRISGSLLTVAFQILVNSLPTAKLTVESVETVNNLISCTVQKIKAALQCES

SEQ ID NO: 12 - DNA, 2466 bp, Lycopersicon esculentum
atgaattttgggggttttcttgataataattctggtggtggcggtgcaagaattgttgctgatataccatttaatcacaacaacagcagcagcaacaacgacaataaaaacaatatgcccactggtgcaatttctcagccaagacttcttcctcaatctcttgccaagaacatgttcaactccccaggactttctcttgctcttcaaacaggtatggaagggcaaagtgaggtaacaagaatggctgaaaactacgagggaaataacagtgttggtagaaggagtagagaagaagaaccagacagcagatctggaagtgataacttggaaggtgcatcgggtgatgaacaagatgctaccgacaaaccaccaaggaagaaaagataccaccgacacactcctcagcaaatccaagaacttgaatctcttttcaaggagtgtcctcatcctgatgagaaacaaaggttggagttaagcaaaaggctttctttggagactagacaagtcaagttttggttccaaaatcgtagaactcagatgaagactcaactggaacgccatgagaattcgatactaaggcaagagaatgataagcttcgtgcagaaaacatgtctataagagaggcaatgagaaatccaatttgtactaattgtggtggtccagcaatgattggtgagatctctcttgaggagcaacatcttaggattgaaaatgctaggctgaaagatgaattagatcgagtttgtgcacttgctggcaagtttttggggcgacccatttcatctttagtaacctctatgcctcccccaatgcctaattcaagtttggaacttggagttggaagtaatggatttggtggtatgagtaatgtaccaacaacactccccttagcacctcctgatttcggtgttgggatttcaaattctttaccagttgtgccttcaactagacagtcaactggaattgagagatcacttgaaagatccatgtatcttgaacttgctttggctgccatggaggaattggtaaaaatggcacaaactgatgaacctctttggttcaggagcattgaaggtggtagagaaatacttaaccatgaagagtacataaggacatttactccttgcattggtatgagaccaaacagttttatttcagaggcttccagggagacaggcatggttataatcaatagtttagctcttgttgagacattaatggactctaacaaatgggcagaaatgtttccatgtttgattgctagaacctcaacaacagatgttatatcaagtggtatgggtggaaccagaaatggtgcacttcagctgatgcatgctgaactccaagtgctttcaccattagtaccaattagagaggtcaatttcctgcgtttctgcaaacaacatgctgaaggtgtctgggctgtcgttgatgtatcgattgataccatccgtgaaacttctggtgcaccaacgtttccaaatagcagaaggcttccttctggctgtgttgttcaagatatgcccaatggctacagcaaagttacatgggtagaacatgccgaatatgaagagggtgcaaatcaccatctttaccggcagttaattagcgcgggtatggggtttggcgcacaaagatgggttgcaaccctccaacgccagtgtgagtgtcttgcaattctcatgtcatccaccgtgtctgccagggatcatacagcaataactccaagtgggaggcgtagcatgttgaagctagcacaacgcatgacaaacaacttttgtgctggtgtttgtgcttcaacggtacacaagtggaacaaactttgtgcaggtaatgttgatgaagatgtacgtgtcatgactcgaaagagcgtcgatgaccctggtgaaccagcagggatcgtcttaagcgctgctacttccgtttggttgcccgtttcccctcaaagactctttgatttcctccgtgacgaacgcctccgtagcgaatgggatatcctatccaacggtggccctatgcaagaaatggctcatattgccaaaggccaagatcatggcaactgcgtctctctccttcgtgctagtgctatgaacgcgaatcagagcagcatgttgatacttcaggagacttgcatagacgcggctggggcgcttgttgtatacgcgccagttgatattccggcaatgcacgtggtgatgaacggtggagattcggcttacgtggctttgcttccttcgggattctcaattgtacccgacggcccaggatctcgtgggtccaatgggccttcatgtaatgggggcccagatcaaagaattagcgggtcactcttgacggtagcctttcagatattggtgaatagtcttcctacagccaagcttactgtggaatcagtggaaacagtcaataatcttatatcatgcactgttcagaagattaaagctgcacttcaatgcgaaagctaa

SEQ ID NO: 13 - DNA, 2466 bp, Lycopersicon esculentum
atgaattttgggggttttcttgataataattctggtggtggcggtgcaagaattgttgctgatataccatttaatcacaacaacagcagcagcaacaacgacaataaaaacaatatgcccactggtgcaatttctcagccaagacttcttcctcaatctcttgccaagaacatgttcaactccccaggactttctcttgctcttcaaacaggtatggaagggcaaagtgaggtaacaagaatggctgaaaactacgagggaaataacagtgttggtagaaggagtagagaagaagaaccagacagcagatctggaagtgataacttggaaggtgcatcgggtgatgaacaagatgctaccgacaaaccaccaaggaagaaaagataccaccgacacactcctcagcaaatccaagaacttgaatctcttttcaaggagtgtcctcatcctgatgagaaacaaaggttggagttaagcaaaaggctttctttggagactagacaagtcaagttttggttccaaaatcgtagaactcagatgaagactcaactggaacgccatgagaattcgatactaaggcaagagaatgataagcttcgtgcagaaaacatgtctataagagaggcaatgagaaatccaatttgtactaattgtggtggtccagcaatgattggtgagatctctcttgaggagcaacatcttaggattgaaaatgctaggctgaaagatgaattagatcgagtttgtgcacttgctggcaagtttttggggcgacccatttcatctttagtaacctctatgcctcccccaatgcctaattcaagtttggaacttggagttggaagtaatggatttggtggtatgagtaatgtaccaacaacactccccttagcacctcctgatttcggtgttgggatttcaaattctttaccagttgtgccttcaactagacagtcaactggaattgagagatcacttgaaagatccatgtatcttgaacttgctttggctgccatggaggaattggtaaaaatggcacaaactgatgaacctctttggttcaggagcattgaaggtggtagagaaatacttaaccatgaagagtacataaggacatttactccttgcattggtatgagaccaaacagttttatttcagaggcttccagggagacaggcatggttataatcaatagtttagctcttgttgagacattaatggactctaacaaatgggcagaaatgtttccatgtttgattgctagaacctcaacaacagatgttatatcaagtggtatgggtggaaccagaaatggtgcacttcagctgatgcatgctgaactccaagtgctttcaccattagtaccaattagagaggtcaatttcctgcgtttctgcaaacaacatgctgaaggtgtctgggctgtcgttgatgtatcgattgataccatccgtgaaacttctggtgcaccaacgtttccaaatagcagaaggcttccttctggctgtgttgttcaagatatgcccaatggctacagcaaagttacatgggtagaacatgccgaatatgaagagggtgcaaatcaccatctttaccggcagttaattagcgcgggtatggggtttggcgcacaaagatgggttgcaaccctccaacgccagtgtgagtgtcttgcaattctcatgtcatccaccgtgtctgccagggatcatacagcaataactccaagtgggaggcgtagcatgttgaagctagcacaacgcatgacaaacaacttttgtgctggtgtttgtgcttcaacggtacacaagtggaacaaactttgtgcaggtaatgttgatgaagatgtacgtgtcatgactcgaaagagcgtcgatgaccctggtgaaccagcagggatcgtcttaagcgctgctacttccgtttggttgcccgtttcccctcaaagactctttgatttcctccgtgacgaacgcctccgtagcgaatgggatatcctatccaacggtggccctatgcaagaaatggctcatattgccaaaggccaagatcatggcaactgcgtctctctccttcgtgctagtgctatgaacgcgaatcagagcagcatgttgatacttcaggagacttgcatagacgcggctggggcgcttgttgtatacgcgccagttgatattccggcaatgcacgtggtgatgaacggtgtagattcggcttacgtggctttgcttccttcgggattctcaattgtacccgacggcccaggatctcgtgggtccaatgggccttcatgtaatgggggcccagatcaaagaattagcgggtcactcttgacggtagcctttcagatattggtgaatagtcttcctacagccaagcttactgtggaatcagtggaaacagtcaataatcttatatcatgcactgttcagaagattaaagctgcacttcaatgcgaaagctaa

SEQ ID NO: 14 - DNA, 9001 bp, Lycopersicon esculentum
  exon (2510)..(2713): Exon 1
  exon (2791)..(3011): Exon 2
  exon (3109)..(3224): Exon 3
  exon (3318)..(4003): Exon 4
  exon (4126)..(4226): Exon 5
  exon (4328)..(4541): Exon 6
  exon (5926)..(6100): Exon 7
  exon (6227)..(6595): Exon 8
  exon (7051)..(7430): Exon 9
tatttttttgttaatgtatatatatttttgttacagtgtatctatactttgttgaatgacttaaacaaaaatattaataatatcaagtggaataattccgaataacaaccattatgcctcgacattgcatcagaacgattgaagtaaaacaataatagggacaaaaattcattagtaacatattttttcttgtttatgtatttaaacgattgtgttaattcatactaataatggtgatgggagagagatgacatctaaatttattaaaattttaataagtagttaataaaatattctgaatactaatataataagtgataattatgtagaaataatatttcaaaattcattacgtgttttgagattattttttttaataattattgtgacacaagaaggtggagagagggggttgcagttcgacaaaagctcaaatgattggttgaatgtatagtacatttatatatggtaggcttgatttataaataaataggtgatgtattggatttattattttttcttgaattgttatattcatcgagaagagttagaattcacgaaagaattttagtatgataaagatggaatgactagttaaatatctagacaataatatagtccacgtgttcattcatctccaaataaataggcgatgcactattgttgttgatttctttgacttggacatatgcattaggaaggaatgaaatacttgaaagggtttcgagttgacaaatatagaatgactgcttgaaggttcgatacgataaatagtagatgtgtccgctccgacttttaaaaaaataggcgatgcatcatctttgctatttttcctaaactagacatgttcattagaaagagtaacaattaaagagtttcaataatatataatagacgtgttcgtttcaaattatgaaataaatagacaatgcatcatcttcacaaatttccttgattaaacacatttattaagaaaagttagaattcataaaaggatttcaatttgacagatatagaacaactcgttgaagatctattgtgaaatataatagacgtgttcgttccgactttcaaataaatatgtttgacttaaaacacattcgttaagaaggatagaaatggaccagctacaagtgggtgaaaaagcccaaatcagaaccacagatccatctatttttgggcccaatataactaaaaacagaaaaaagccatcccagagaaaaggagtaataaacgtcagcagttgacgccggcgagtcaccggccatttgactcgccggattccggtaataacaaatacagttgtcaaatatgaatacgacccgcgtacacacaggtcaagtgggtcaaaacaaaaccttagtgatagaagaaaagaaattaaaaaattaaggagaaagaatctttttttcctcaatatttatgattattttcctttaaattaatctagtgataaacccattgactaataaacccttgattcctgtatgaaatttgtcaccttgctgtaattaattcatataatcttcatgtaaatttcagatcccaatatagtaaaaagataaagaagaaattttgtgagtgtaaaaaaacaaaatcttgggatctgaatgaactgtctttactctggcatttcattttcttcttgtgttaaatctctgtttgttttttttacagtaaaaagtttaaactgtgaaaaaaaaaaaaagctacacattttttcacagtttgacggtagatttcaagaattagagagaggggttgggggttgaaggggagtggggtgggggtgaaagagtcaaccaaacctttttctgtgctgttttggctgtatgtatatgtaaggcttcgatgagtccacaattttgcgtattatcttcacctttttttttttggtttttagtattatttttttctactgttgttacccctttttctatctctcgtctcttctattgtctttgcaaaactgattaaacaaagcctttttagcttctttttctctcacttccttttctgtgctttattttttatttttttggtttacaaaagtcaagcatgcagagatacatacatataacagaacagaaaaatgccctctaacttagggttttgcttgcttaaaccattcttttaagtatctatgcttttgtatttgcagttaatgattagtttaagaaaggtcccttttttttttttactgttttgttaggagggagagaagagaaattttaattagtactgttatttaaggaaagtatagagaagagaaaggcaaagagtagcacaagttaagggttttttttttttggctttcttttcttgttatttttctttcaagagtaaaaattagagaggaagttggagacatttggattcgtccttgtgtctcctctacctctcataatttttctcccaactttttagctcaaaaaagatacagtgaatctgtcaaatatacaaaaaaagaaaaagtttagttctttttgagtttctcttgctttatttcatgaattttgggggttttcttgataataattctggtggtggcggtgcaagaattgttgctgatataccatttaatcacaacaacagcagcagcaacaacgacaataaaaacaatatgcccactggtgcaatttctcagccaagacttcttcctcaatctcttgccaagaacatgttcaactccccaggactttctcttgctcttgtaagccaaatgaaaaagagcataaaaattgaatcttttttacttttatatgacacccttttttttttttgttgcagcaaacaggtatggaagggcaaagtgaggtaacaagaatggctgaaaactacgagggaaataacagtgttggtagaaggagtagagaagaagaaccagacagcagatctggaagtgataacttggaaggtgcatcgggtgatgaacaagatgctaccgacaaaccaccaaggaagaaaagataccaccgacacactcctcagcaaatccaagaacttgaatcgtaatcattaccttctttttcaaatgaaaaaaaaatgatttttttttctttcaacttttcttatgagatgtttctattatggttttttttttcttagtcttttcaaggagtgtcctcatcctgatgagaaacaaaggttggagttaagcaaaaggctttctttggagactagacaagtcaagttttggttccaaaatcgtagaactcagatgaaggtaataaaaaaaagattcaatttctatggctttaaaagattctttttttttgtgggtttctgcttctgaataatgaatctttgattgcaccagactcaactggaacgccatgagaattcgatactaaggcaagagaatgataagcttcgtgcagaaaacatgtctataagagaggcaatgagaaatccaatttgtactaattgtggtggtccagcaatgattggtgagatctctcttgaggagcaacatcttaggattgaaaatgctaggctgaaagatgaattagatcgagtttgtgcacttgctggcaagtttttggggcgacccatttcatctttagtaacctctatgcctcccccaatgcctaattcaagtttggaacttggagttggaagtaatggatttggtggtatgagtaatgtaccaacaacactccccttagcacctcctgatttcggtgttgggatttcaaattctttaccagttgtgccttcaactagacagtcaactggaattgagagatcacttgaaagatccatgtatcttgaacttgctttggctgccatggaggaattggtaaaaatggcacaaactgatgaacctctttggttcaggagcattgaaggtggtagagaaatacttaaccatgaagagtacataaggacatttactccttgcattggtatgagaccaaacagttttatttcagaggcttccagggagacaggcatggttataatcaatagtttagctcttgttgagacattaatggactctgtaagtaatttcttgattgcacttttaggtgtctaatcctgtaaatatccttgtcaattatcatacatgaaagtcataaatcttcatttgttttttctgatgattagctttaattttgacagaacaaatgggcagaaatgtttccatgtttgattgctagaacctcaacaacagatgttatatcaagtggtatgggtggaaccagaaatggtgcacttcagctggtaaattaaaatgaacctttgtttgtagttaaatcaaatgttattattattattattagtcaatgttgaaatatgattttttttttcttgattctttatagatgcatgctgaactccaagtgctttcaccattagtaccaattagagaggtcaatttcctgcgtttctgcaaacaacatgctgaaggtgtctgggctgtcgttgatgtatcgattgataccatccgtgaaacttctggtgcaccaacgtttccaaatagcagaaggcttccttctggctgtgttgttcaagatatgcccaatggctacagcaaagtaagttctctatgaataaattcaattgtctatattcggttaactctattcacaaagacttttctctcttctactagtgtgtcgtttagtactatatattcaatgagtttacattcatggggtagctactattttgctttttgtgtttaatgagtttggttgagcaactacaaatttttggatcttagttcagaacattgatttggttttttttccccacctaatttggattgtcaaaatgactaaaagctttagtagtcttttttggttatttggtccaaaaccaaaaagattagattattgaacctgataagatgctgattataggggacataacagtattaaaaagtcatcaaatatcagcaacttacttcctgcttaagatgagcttaatactaatattatatgaacaccgtacgtgtggaaatcgaggttaaggacgagttaattaatctaataaattgaatagaagtctccattacttatatttttgtgccttgggacacattaatttcatatgattcttcagaaaataatttaaaaaaaattaaattaaaaatttgctttgtaactttcaagcattggtttaattaaacaatgtatttgatgtttctattccaatctctgatgctgtctcgatcagtcccccgtgtatataattaattcatctaactgtgtattatctggttccagccttgtgatgtgtttgttttttatactatactaatgaaggactctttttagtcaaacaacaagactgactacattattttttgttgtcacgtgagtatctacagagtctaaaagctttgtggtacatttctcaatgtctcaaacctaacctcgtgccttcaaatttgcataaaaatggtgaaaacttgtatttttatgacttctaattttagggttctaggttctatcatgtctctccttgaacctaactttgctatagaaaccatttccaagatttgtcctgttgcatcacaaaaatttcccatttagtaatgtacttatttttttattctttttctaatttttggggtttttaaaacatgtttggattaaaataaatctatactaatagcactaataagttaggtgacgttattcatggtctaaatgtttgaattcatttgtcacaactaactttgctcaggagttgtaattatttgcctatgtgataaatacttgtcataaattactctataatttaaatgtagtcgataataaattcaatgcatatgatcattaatttactatgtaacgagggaaaattgtgacaacaattgattaactccccttgtgattattattattatgctctaatttattccaagcaagttcagaccgactaaataaattctgacggttaataattatgtaggttacatgggtagaacatgccgaatatgaagagggtgcaaatcaccatctttaccggcagttaattagcgcgggtatggggtttggcgcacaaagatgggttgcaaccctccaacgccagtgtgagtgtcttgcaattctcatgtcatccaccgtgtctgccagggatcatacaggtaactaattaaaatctatatttttgtgaaactgttcaatgaatcaacctctatcatgtgatgagtagtagacccttatattttgtgtgaccaaatttaaattttaagaaatcaaatcacatgcagcaataactccaagtgggaggcgtagcatgttgaagctagcacaacgcatgacaaacaacttttgtgctggtgtttgtgcttcaacggtacacaagtggaacaaactttgtgcaggtaatgttgatgaagatgtacgtgtcatgactcgaaagagcgtcgatgaccctggtgaaccagcagggatcgtcttaagcgctgctacttccgtttggttgcccgtttcccctcaaagactctttgatttcctccgtgacgaacgcctccgtagcgaatgggatatcctatccaacggtggccctatgcaagaaatggctcatattgccaaaggccaagatcatggcaactgcgtctctctccttcgtgctagtgtaatttcctcaaccctttcttttttcttttttttttttatgaaaaaattaattttgaatggatagagataatgcatggcgatttatacatatgaatgcatgtaaaaagattgattgattatgcatgtgtggggttcattttcttgtaagataaaaataaaattgttgattgaaaaggacaaactagggggatttggaagtatctcaaaaggaccatcttattccagatctataaatttttaactttgttgcatcttttgtaaatttgtaaaaagtcaaaacacatgatacatgttacctttttattttagtcagccctactatgatatcaccctttttacatgaaaaagtcttcaaacactgcaacaaacaactgacgtattatatataccacacaatggcattattcgtaaatatattcataaccaaggggctttgtattgatctattgccaggctatgaacgcgaatcagagcagcatgttgatacttcaggagacttgcatagacgcggctggggcgcttgttgtatacgcgccagttgatattccggcaatgcacgtggtgatgaacggtggagattcggcttacgtggctttgcttccttcgggattctcaattgtacccgacggcccaggatctcgtgggtccaatgggccttcatgtaatgggggcccagatcaaagaattagcgggtcactcttgacggtagcctttcagatattggtgaatagtcttcctacagccaagcttactgtggaatcagtggaaacagtcaataatcttatatcatgcactgttcagaagattaaagctgcacttcaatgcgaaagctaatatttgagacaaggagaacaactttttttaatatatttttttttaataatatatagtttaactagtagaaagaaaaaaaaaaagtggtgcggtaatggaaggagtggaggcttgatcattgaagaactcgagctgaattggaggctgagtcaagaacgaaccgcgctgaaaagaccattgctaggtggtaagggccggctcaactagctcgaggtgttattaatttggtgttcaacacaaagcaaaggtggtggttcgggtattgactcagggtactaatatcagttggttcatgaatgaatcagcttttgggtgaaaattttcaatttggacaagttgtgtgacagaattatgtcaatttcagaagctatggtttgtttttgatgaatttatagttgtttgttgaagagtcataatggctcttttgttttgtatgttatggtgggggacaattgacaaatcaactattgtacgttatcttaattgacctactttaacttgaatctcattgtcattgtttatttttctttgccattatggtctaggttcacatttgatcacaagtgaaaatttgttttattttaacttttacagtttatcaagtcgtaatatatatatctttctgaatattgtatgttcgatatctttgaaagttatgtatgtcatcttcaataaggattaaaccatcgctcttcattcttgttttgcagattaattaattgttactcagacaaaatatgcgactatcatgtgataatataagcattgtaatctaaagagaaatttcattcacatgtatattttaagaaatctgcaaaaattcacgtactacatcaattttttataaaattctaatttatttatcttaatgtaatcctaatatcttacctgaaataaataaaataaataggaaatattatgaggtatttattgattttaggcttacgttgtttgatgtacatgtatttgctaagttcgtgaagatattaggtgaattatatggacaatacaaatataacattattctgtaaaaaactgttattcttctttgataaaaaaataaatttatattaaaagtttattaaaacgtgtgataaatatattatatattagtaaaatttatattatacatataaattattttatataatatgtattaaaacaatatgatgaatatttcatagaaaaatattattgctataaataacgaatatttttttaatctaaaatatctatttaaatttttcaaaagatatttacgtaatcgaatgttaaagttaatcaaataagattgatttttttttaaagaaaaagttagggcaggattagagatgaagttaaagagaaagggaatgggtttgtgaattgtgatgagtgaaggtgaagacagctgaggagtgatggagaaagggggcgggggctcctgcaaagtgaggtgcatttaatgtaatttcagaagctgttgggacaaacacttcccatgcatttaatgctctctttcctatacttatttgtattaaagtctgattaaatttaaatcttcatatattaagagataaaatgctttttaa

SEQ ID NO: 15 - protein, 821 aa, Lycopersicon esculentum
MNFGGFLDNNSGGGGARIVADIPFNHNNSSSNNDNKNNMPTGAISQPRLLPQSLAKNMFNSPGLSLALQTGMEGQSEVTRMAENYEGNNSVGRRSREEEPDSRSGSDNLEGASGDEQDATDKPPRKKRYHRHTPQQIQELESLFKECPHPDEKQRLELSKRLSLETRQVKFWFQNRRTQMKTQLERHENSILRQENDKLRAENMSIREAMRNPICTNCGGPAMIGEISLEEQHLRIENARLKDELDRVCALAGKFLGRPISSLVTSMPPPMPNSSLELGVGSNGFGGMSNVPTTLPLAPPDFGVGISNSLPVVPSTRQSTGIERSLERSMYLELALAAMEELVKMAQTDEPLWFRSIEGGREILNHEEYIRTFTPCIGMRPNSFISEASRETGMVIINSLALVETLMDSNKWAEMFPCLIARTSTTDVISSGMGGTRNGALQLMHAELQVLSPLVPIREVNFLRFCKQHAEGVWAVVDVSIDTIRETSGAPTFPNSRRLPSGCVVQDMPNGYSKVTWVEHAEYEEGANHHLYRQLISAGMGFGAQRWVATLQRQCECLAILMSSTVSARDHTAITPSGRRSMLKLAQRMTNNFCAGVCASTVHKWNKLCAGNVDEDVRVMTRKSVDDPGEPAGIVLSAATSVWLPVSPQRLFDFLRDERLRSEWDILSNGGPMQEMAHIAKGQDHGNCVSLLRASAMNANQSSMLILHETCIDAAGALVVYAPVDIPAMHVVMNGGNSAYVALLPSGFSIVPDGPGSRGSNGPSCNGGPDQRISGSLLTVAFQILVNSLPTAKLTVESVETVNNLISCTVQKIKAALQCES

SEQ ID NO: 16 - DNA, 2466 bp, Lycopersicon esculentum
atgaattttgggggttttcttgataataattctggtggtggcggtgcaagaattgttgctgatataccatttaatcacaacaacagcagcagcaacaacgacaataaaaacaatatgcccactggtgcaatttctcagccaagacttcttcctcaatctcttgccaagaacatgttcaactccccaggactttctcttgctcttcaaacaggtatggaagggcaaagtgaggtaacaagaatggctgaaaactacgagggaaataacagtgttggtagaaggagtagagaagaagaaccagacagcagatctggaagtgataacttggaaggtgcatcgggtgatgaacaagatgctaccgacaaaccaccaaggaagaaaagataccaccgacacactcctcagcaaatccaagaacttgaatctcttttcaaggagtgtcctcatcctgatgagaaacaaaggttggagttaagcaaaaggctttctttggagactagacaagtcaagttttggttccaaaatcgtagaactcagatgaagactcaactggaacgccatgagaattcgatactaaggcaagagaatgataagcttcgtgcagaaaacatgtctataagagaggcaatgagaaatccaatttgtactaattgtggtggtccagcaatgattggtgagatctctcttgaggagcaacatcttaggattgaaaatgctaggctgaaagatgaattagatcgagtttgtgcacttgctggcaagtttttggggcgacccatttcatctttagtaacctctatgcctcccccaatgcctaattcaagtttggaacttggagttggaagtaatggatttggtggtatgagtaatgtaccaacaacactccccttagcacctcctgatttcggtgttgggatttcaaattctttaccagttgtgccttcaactagacagtcaactggaattgagagatcacttgaaagatccatgtatcttgaacttgctttggctgccatggaggaattggtaaaaatggcacaaactgatgaacctctttggttcaggagcattgaaggtggtagagaaatacttaaccatgaagagtacataaggacatttactccttgcattggtatgagaccaaacagttttatttcagaggcttccagggagacaggcatggttataatcaatagtttagctcttgttgagacattaatggactctaacaaatgggcagaaatgtttccatgtttgattgctagaacctcaacaacagatgttatatcaagtggtatgggtggaaccagaaatggtgcacttcagctgatgcatgctgaactccaagtgctttcaccattagtaccaattagagaggtcaatttcctgcgtttctgcaaacaacatgctgaaggtgtctgggctgtcgttgatgtatcgattgataccatccgtgaaacttctggtgcaccaacgtttccaaatagcagaaggcttccttctggctgtgttgttcaagatatgcccaatggctacagcaaagttacatgggtagaacatgccgaatatgaagagggtgcaaatcaccatctttaccggcagttaattagcgcgggtatggggtttggcgcacaaagatgggttgcaaccctccaacgccagtgtgagtgtcttgcaattctcatgtcatccaccgtgtctgccagggatcatacagcaataactccaagtgggaggcgtagcatgttgaagctagcacaacgcatgacaaacaacttttgtgctggtgtttgtgcttcaacggtacacaagtggaacaaactttgtgcaggtaatgttgatgaagatgtacgtgtcatgactcgaaagagcgtcgatgaccctggtgaaccagcagggatcgtcttaagcgctgctacttccgtttggttgcccgtttcccctcaaagactctttgatttcctccgtgacgaacgcctccgtagcgaatgggatatcctatccaacggtggccctatgcaagaaatggctcatattgccaaaggccaagatcatggcaactgcgtctctctccttcgtgctagtgctatgaacgcgaatcagagcagcatgttgatacttcatgagacttgcatagacgcggctggggcgcttgttgtatacgcgccagttgatattccggcaatgcacgtggtgatgaacggtggaaattcggcttacgtggctttgcttccttcgggattctcaattgtacccgacggcccaggatctcgtgggtccaatgggccttcatgtaatgggggcccagatcaaagaattagcgggtcactcttgacggtagcctttcagatattggtgaatagtcttcctacagccaagcttactgtggaatcagtggaaacagtcaataatcttatatcatgcactgttcagaagattaaagctgcacttcaatgcgaaagctaa

(19) United States
(12) Patent Application Publication
(10) Pub. No.: US 2017/0359979 A1
(43) Pub. Date: Dec. 21, 2017

(54) SOLANUM LYCOPERSICUM PLANTS HAVING PINK GLOSSY FRUITS

(71) Applicant: NUNHEMS B.V., NUNHEM, NL (US)

(72) Inventors: Hendrik Willem VRIEZEN, Haelen (NL); Henricus Maria VERBAKEL, Boekel (NL)

(21) Appl. No.: 15/329,934

(22) PCT Filed: Jul. 28, 2015
(86) PCT No.: PCT/EP2015/067200

§ 371 Date: Jan. 27, 2017

(30) Foreign Application Priority Data
Jul. 30, 2014 (EP) 14179056.8

(51) Int. Cl. A01H 5/08; A01H 1/02

(52) U.S. Cl. CPC A01H 5/08; A01H 1/02

(57) ABSTRACT

The present invention relates to cultivated plant of the species Solanum lycopersicum comprising a myb12 allele having one or more mutations, said mutations resulting in production of a mutant myb12 protein, fruits of such plants exhibiting a pink appearance and in addition comprising a mutant cuticle deficiency (cd) allele, whereby the fruits become glossy.

### FIELD OF THE INVENTION

This invention relates to the field of plant biotechnology and plant breeding. Provided are cultivated Solanum lycopersicum plants producing pink glossy fruits (i.e. fruits which are pink and glossy in appearance), comprising a myb12 allele (myeloblastosis allele number 12) comprising one or more mutations, and additionally comprising a mutation in an allele involved in cuticle development, such as in an allele encoding a CD protein (Cutin Deficient or Cutin Deficiency protein), said mutation resulting in an (significantly) increased glossiness of the fruits compared to wild type and/or an (significantly) increased or decreased accumulation of cutin at Red Ripe (RR) stage. In one aspect the increased or decreased accumulation of cutin at Red Ripe (RR) stage is an increase or decrease of at least 15% compared to a plant lacking the mutation in an allele involved in cuticle development (such as an allele in a CD gene, encoding a CD protein) i.e. an at least a 15% thicker or an at least 15% thinner cutin layer thickness, respectively. In one aspect a cultivated tomato plant comprises a myb12 allele (also referred to as pink allele) in homozygous form and further comprises a cd-allele (also referred to as glossy allele or cutin deficiency allele) in homozygous or heterozygous form, especially cd2/cd2 (homozygous) or CD2/cd2 (heterozygous), whereby the red-fleshed fruits of said plants have a colorless peel and therefore a pink appearance in addition to a glossy appearance. The heterozygous form of the glossy mutant cd2 showed an increased glossiness compared to plants lacking the mutant (homozygous for wild type CD2/CD2).

The invention further provides plants of the invention comprising a myb12 allele having one or more mutations, said mutations resulting in production of a mutant myb12 protein, wherein said mutant myb12 protein has a G50R amino acid substitution in SEQ ID NO: 1, or in variants thereof having at least 85% amino acid sequence identity to SEQ ID NO: 1; or wherein said mutant myb12 protein comprises a deletion of the amino acids 61 to 338 in SEQ ID NO: 1, or in variants thereof, said variants having at least 85% amino acid sequence identity to SEQ ID NO: 1, or wherein the plant comprises the y (yellow) gene. In addition, the plants comprising the mutant myb12 protein preferably also comprise an allele encoding a mutant CD protein, e.g. a mutant allele of CD1, CD2 or CD3 protein. In one aspect the mutant CD protein is the protein of SEQ ID NO: 11, comprising a G736V amino acid substitution relative to the wild type CD2 protein (SEQ ID NO: 2); and/or the mutant CD2 protein of SEQ ID NO: 15, comprising a D737N and/or a Q708H amino acid substitution relative to the wild type CD2 protein as shown in SEQ ID NO: 10. Thus, in one aspect the tomato plants produce fruits which are pink glossy and which cells are homozygous for myb12/myb12 (mutant pink alleles) or y/y (the yellow allele) and homozygous or heterozygous for a mutant cd allele selected from cd1, cd2 and cd3 (mutant glossy alleles, e.g. CD2/cd2 or cd2/cd2; CD1/cd1 or cd1/cd1; CD3/cd3 or cd3/cd3). The invention also provides tomato seeds from which the plants according to the invention can be grown and/or from which a mutant myb12 gene and/or a mutant cd2 gene according to the invention can be obtained and introduced into any other tomato plant by traditional breeding, in order to generate other tomato plants producing pink glossy fruits. Food and food products comprising or consisting of fruits of the plants of the invention are provided too. Also, methods of producing plants, seeds, plant tissues and cells according to the invention and/or food and/or feed products made from these, from any other tomato plant, seed, tissue, cell, food or feed, are encompassed herein, whereby the presence of the mutant myb12 and/or cd2 gene, mRNA (cDNA) and/or protein is detectable.

### BACKGROUND OF THE INVENTION

Skin color in tomato fruit is determined by the Y gene. Gene Y produces a distinct, yellow pigment suffused throughout the cell walls of the epidermis of the fruit, whereas its allelomorph, y gene, produces a transparent or colorless condition in the epidermal walls (E. W. Lindstrom, 1925, Inheritance in Tomatoes, Genetics, issue 10(4) pp 305-317).

Pink tomato fruit is very popular for consumption in Asia. The pink fruit was first described in fruit with a transparent epidermis lacking a yellow pigment (Lindstrom, 1925, Inheritance in Tomatoes, Genetics, issue 10 (4) pp 305-317). Genetic studies revealed that pink fruit result from the monogenic recessive y (yellow) locus present on chromosome 1, while red-colored fruit have the dominant Y allele (Lindstrom 1925). The Y gene has been identified as MYB12 (Ballester et al, vide infra). Many tomato accessions are available which comprise the y gene, see e.g. the world wide web at tgrc.ucdavis.edu/data/acc/GenDetail.aspx?gene=y.

The color of tomato fruit is mainly determined by carotenoids and flavonoids. The red color of ripe tomato fruit is due mainly to the accumulation of the carotenoid all-trans-lycopene, which is produced during fruit ripening. In addition to lycopene, tomato fruit contain significant levels of violaxanthin, and lutein. Tomato plants having mutation(s) in the carotenoid pathway have an altered carotenoid composition, which result in different fruit colors, such as orange (tangerine beta) or yellow (r) fruit (Lewinsohn et al. 2005 Trends Food Sci Technol., Vol 16 pp 407-415).

Additionally flavonoids play a role in determining the color of tomato fruit. Flavonoids accumulate predominantly in the fruit peel, since the flavonoid pathway is not active in the fruit flesh. One of the most abundant flavonoids in tomato fruit peel is the yellow-colored naringenin chalcone. In addition, up to 70 different flavonoids have been identified in tomato fruit.

Ballester et al. performed a phenotypic analysis of an introgression line (IL) population derived from a cross between Solanum lycopersicum “Moneyberg” and the wild species Solanum chmielewskii which revealed three ILs with pink fruit color. These ILs had a homozygous S. chmielewskii introgression on the short arm of chromosome 1, consistent with the position of the y (yellow) mutation known to result in colorless epidermis, and hence pink-colored fruit when combined with a red fruit flesh. This same study revealed that the pink fruit lacked the ripening-dependent accumulation of the yellow-colored flavonoid naringenin chalcone in the fruit peel, which increased in the peel of Moneyberg fruit upon ripening, while carotenoid levels were not affected (Ballester et al. 2010 Plant Physiology, vol 152 pp 71-84). In the same study Ballester et al. disclose (Ballester et al. 2010 Plant Physiology, vol 152 pp 77 right-hand column) that “the deduced amino acid sequence of the pink MYB12 alleles obtained from commercial sources was identical to the red Moneyberg allele”, suggesting “that deregulated MYB12 gene expression, observed in all pink genotypes tested [by Ballester et al.], rather than aberrant MYB12 [protein] function is the primary cause of the pink phenotype. This cause of the pink color was confirmed using gene-silencing studies genetic mapping, segregation analysis, and VIGS (Virus Induced Gene Silencing) results.

Thus far, analysis of existing commercial non-GMO colorless peel (i.e. pink) y mutant revealed no mutations in the myb12 allele nor in its promotor sequence indicating that the y mutant phenotype is due to a mutation in a regulatory gene i.e. an additional mutant allele (Adato et al 2009 PLoS Genetics, vol 5, issue 12, e1000777).

In PCT/EP2014/051582 two non-GMO, cultivated pink Solanum lycopersicum plants were disclosed having a mutant myb12 protein, wherein said mutant myb12 protein has a G50R amino acid substitution compared to the wild type Solanum lycopersicum myb12 protein sequence or wherein said mutant myb12 protein comprises a deletion of the amino acids 61 to 338 in SEQ ID NO: 1. The Pink fruits (due to the colorless peel) of these non-GMO cultivated tomato plants are dull in appearance when compared to wild type (i.e. red) cultivated tomato plants, just like the pink fruits from plants being homozygous for the monogenic recessive y (yellow) locus. These fruits thus appear to be less glossy. As a result, pink non-GMO cultivated tomato fruits have a less attractive appearance when compared to fruits of wild type cultivated tomato plants, which are glossy.

The plant cuticle is a protective layer consisting of cutin and filled with waxes which also accumulate on the surface. It is synthesized by plant epidermal cell walls. Plant cuticles play an important role in restricting water loss from aerial plant organs, control of pathogens, cracking, and postharvest shelf-life. Tomato fruit brightness (also referred to as glossiness) is also controlled by tomato fruit cuticle and seems independent from wax load. However, so far no obvious link could be made between cutin load and/or composition and fruit brightness. Tomato plants with fruits with an altered cutin layer may appear to be either dull or glossy. It appears that many genes are involved in cutin development. These genes may map to different chromosomes and may even have different inheritance patterns (Petit et al Plant Physiology, 2014, Vol 164, pp 888-906; Isaacson et al The Plant Journal, 2009, Vol 60, pp 363-377).

There is, thus, a need for non-GMO, cultivated tomato plants producing pink tomato fruits that are more glossy (i.e. glossier) than fruits of normal non-GMO, cultivated tomato plants producing pink tomato fruits.

### SUMMARY OF THE INVENTION

It was surprisingly found by the inventors that cultivated plants of species Solanum lycopersicum having an aberrant MYB12 protein function, instead of a deregulated MYB12 gene expression, produced pink-appearing tomato fruits. This was very surprising, in view of Ballester et al. 2010 and Adato et al (2009) (supra) which discloses that a deregulated MYB12 gene expression results in pink-appearing tomato fruit. Unfortunately, these plants having an aberrant MYB12 protein function also have dull (i.e. non-glossy) tomato fruits, like the non-GMO tomato plant comprising the y-gene. However, when cultivated plants of species Solanum lycopersicum having (or capable of producing) pink fruits have an additional mutation in an allele involved in cuticle development, such as in an allele of a Cutin Deficiency gene (CD gene), said mutation results in glossy appearance of the fruits. In one aspect the mutation results in an increased or decreased accumulation of cutin at Red Ripe (RR) stage of the fruits. In one aspect the amount of cutin (cutin content of the cuticle) at the RR stage of the fruit is at least 15% increased or at least 15% decreased compared to a plant lacking the mutation in an allele involved in cuticle development (such as a CD allele) and/or the cuticle layer is at least a 15% thicker or at least 15% thinner, respectively. Such plants produce (are capable of producing) glossy pink tomato fruits. This was very surprising, especially in view of the fact that the plants or fruits did not have any other plant or fruit phenotypic changes or disease susceptibility. The allele involved in cuticle development is in one aspect selected from a tomato CD gene (e.g. a tomato CD1 gene, CD2 gene or CD3 gene), especially from a mutant cd1 allele, cd2 allele or cd3 allele. The amount of cutin and/or the cuticle layer thickness is compared to the normal tomato plants which do not comprise the mutant CD gene, i.e. which comprise wild type CD1, CD2 and CD3 alleles.

The invention thus relates to a cultivated plant of the species Solanum lycopersicum producing pink and glossy fruits (also referred to as ‘pink glossy fruits’) comprising a myb12 allele having one or more mutations and comprising a mutation in an allele involved in cuticle development, said mutation resulting in an increased or decreased accumulation of cutin at Red Ripe (RR) stage compared to the wild type plant (lacking the mutant allele in the gene for cuticle development). In one aspect the increase or decrease in cutin is at least 15% compared to a plant lacking the mutation in an allele involved in cuticle development and/or the cuticle thickness is at least a 15% thicker or an at least 15% thinner, respectively.

The invention also relates to a cultivated plant of the species Solanum lycopersicum producing pink glossy fruits, comprising a myb12 allele comprising one or more mutation in homozygous form or comprising the y (yellow) gene in homozygous form and further comprising a Cuticle Deficiency (CD) allele comprising one or more mutations in homozygous or heterozygous form, said mutant cd-allele resulting in an increased glossiness of the fruits compared to fruits of plants lacking said mutant cd-allele.

In one embodiment the invention relates to a cultivated tomato plant of the invention wherein said mutation or mutations in the genomic myb12 gene result in the fruits of said plant exhibiting a pink appearance at the late orange and red stages of fruit development, preferably combined with a glossy (non-dull) appearance of the fruits due to the presence of a mutant cd-allele, preferably a mutant cd-allele encoding a mutant CD protein. In one aspect the mutant cd-allele is a cd2 allele and the mutation causes one or more amino acid substitutions relative to the wild type (functional) CD2 protein, selected from a G736V, substitution, a D737N substitution and/or a Q708H substitution in SEQ ID NO: 10 or in a CD allele encoding a variant of SEQ ID NO: 10, such as a cd-allele encoding a CD2 protein comprising at least 70%, 80%, 85%, 90%, 95%, 97%, 98% or 99% sequence identity to SEQ ID NO: 10. The one or more mutations selected from a G736V, substitution, a D737N substitution and/or a Q708H substitution are thus in one aspect present in such a variant of SEQ ID NO: 10.

### General Definitions

The term “nucleic acid sequence” (or nucleic acid molecule) refers to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein” or “polypeptide” are used interchangeably and refer to molecules consisting of a chain of amino acids, without reference to a specific mode of action, size, 3-dimensional structure or origin. A “fragment” or “portion” of Myb12 protein may, thus, still be referred to as a “protein”. An “isolated protein” is used to refer to a protein which is no longer in its natural environment, for example in vitro or in a recombinant bacterial or plant host cell.

The term “gene” means a DNA sequence comprising a region (transcribed region), which is transcribed into an RNA molecule (e.g. an mRNA, hpRNA or an RNAi molecule) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. A gene may be an endogenous gene (in the species of origin) or a chimeric gene (e.g. a transgene or cis-gene).

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence may be in sense-orientation and encodes a desired, biologically active protein or peptide, or an active peptide fragment.

An “active protein” or “functional protein” is a protein which has protein activity as measurable in vitro, e.g. by an in vitro activity assay, and/or in vivo, e.g. by the phenotype conferred by the protein. A “wild type” Myb12 protein is a fully functional protein comprising at least about 85%, 90%, 95%, 96%, 97%, 98%, or 99% amino acid sequence identity to SEQ ID NO: 1 (also referred to as “variants” or “functional variants” of SEQ ID NO: 1). Likewise, the wild type Myb12 allele is the allele encoding said wild type protein or wild type functional variant.

A “mutant myb12 protein” is herein a protein comprising one or more mutations in the nucleic acid sequence encoding the wild type Myb12 protein, whereby the mutation results in (the mutant nucleic acid molecule encoding) a “reduced-function” or “loss-of-function” protein, as e.g. measurable in vivo, e.g. by the modified phenotype conferred by the mutant allele. A “reduced function myb12 protein” or “reduced activity myb12 protein” or loss-of-function myb12 protein refers to a mutant myb12 protein which results in a colorless fruit epidermis, or colorless peel, which gives the ripe fruit a pink color when combined with the red tomato fruit flesh.

A “mutant cd protein” is herein a protein comprising one or more mutations in the nucleic acid sequence encoding the wild type CD protein (Cutin Deficiency protein or Cutin Deficient protein), whereby the mutation results in (the mutant nucleic acid molecule encoding) a “reduced-function” or “loss-of-function” protein, as e.g. measurable in vivo, e.g. by the modified phenotype conferred by the mutant allele. A “reduced function cd protein” or “reduced activity cd protein” or loss-of-function cd protein refers to a mutant cd protein which results in glossy appearance of the tomato fruits at red ripe stage and/or a significant increase or decrease of the cutin content and/or cuticle layer thickness of the fruits (of the fruit cuticle). The mutant cd protein may e.g. be a mutant cd1, cd2 or cd3 protein.

“Pink tomato fruit”, “y mutant”, “y phenotype”, or “colorless peel y phenotype/mutant” or “colorless epidermis” or “colorless peel” refers to tomato fruit, or a tomato plant capable of producing fruit, having a less colored (less pigmented; more transparent) fruit epidermis e.g. when compared to the yellow/orange-colored normal epidermis (found in fruits of plants comprising one or two copies of the gene encoding the wild type Myb12 protein of SEQ ID NO:1 or functional variants), which result in pink appearance of the fruit when combined with red flesh. As the myb12 mutation is recessive, only the epidermis of fruits of tomato plants comprising the mutant myb12 allele in homozygous form will have the colorless peel. The color of the fruit epidermis can simply be compared visually, by looking at the fruits at the red stage and/or by peeling off the epidermis and visually assessing the pigmentation of the epidermis by e.g. holding the epidermis against a light source. Alternatively, the total flavonoid content or level of naringenin chalcone can be determined in the fruit peel tissue as described in Adato et al (supra) or Ballester et al. 2010 (supra). In particular, under Materials and Methods—Flavonoid and Carotenoid Extraction and HPLC Analysis, Ballester et al refer to Boni et al (2005) who describe a flavonoid detection method in paragraph “Phenolic and ascorbic acid extraction, separation and detection by HPLC-PDA (page 429, left-hand column of Boni et al (2005) (Boni et al. New Phytologist (2005) volume 166 pp 427-438). The epidermis tissue (peel) of the colorless epidermis myb12 mutants comprises significantly less naringenin chalcone than peel of wild type fruits, e.g. less than 50 mg/kg fresh weight peel, preferably less than 20, 10, 5, 2, or less than 1 mg/kg fw of peel in the fruits homozygous for a mutant myb12 allele. So in one aspect, colorless epidermis” or “colorless peel” is defined as an epidermis comprising less naringenin chalcone than 50 mg/kg fresh weight peel, preferably less than 20, 10, 5, 2, or even less than 1 mg/kg fw of peel in the fruits.

Epidermis refers to a single-layered group of cells that covers plants' leaves, flowers, fruits and stems. It forms a boundary between the plant and the external environment. The epidermis serves several functions, it protects against water loss, regulates gas exchange, secretes metabolic compounds, and (especially in roots) absorbs water and mineral nutrients.

Normal epidermis or epidermis of normal/red-colored tomato-fruit (i.e. of plants comprising the gene encoding the wild type Myb12 protein) has, at the red-stage of ripening, a yellow/orange color due to accumulation of yellow-colored flavonoid naringenin chalcone in the fruit epidermis, like for example in red varieties such as Moneyberg, Pusa Sheetal, Tapa, M82 or TPAADASU, and many other tomato varieties grown in countries other than China.

A reduced function myb12 protein can be obtained by the transcription and translation of a “partial knockout mutant myb12 allele” which is, for example, a wild-type Myb12 allele, which comprises one or more mutations in its nucleic acid sequence. In one aspect, such a partial knockout mutant myb12 allele is a wild-type Myb12 allele, which comprises one or more mutations that preferably result in the production of a myb12 protein wherein at least one conserved and/or functional amino acid is substituted for another amino acid, such that the biological activity is significantly reduced but not completely abolished. However, other mutations, such as one or more non-sense, missense, splice-site or frameshift mutations in the tomato Myb12 allele may also result in reduced function myb12 protein and such reduced function proteins may have one or more amino acids replaced, inserted or deleted, relative to the wild type Myb12 protein. Mutant alleles can be either “natural mutant” alleles, which are mutant alleles found in nature (e.g. produced spontaneously without human application of mutagens) or “induced mutant” alleles, which are induced by human intervention, e.g. by mutagenesis.

A “mutation” in a nucleic acid molecule coding for a protein is a change of one or more nucleotides compared to the wild type sequence, e.g. by replacement, deletion or insertion of one or more nucleotides. A “point mutation” is the replacement of a single nucleotide, or the insertion or deletion of a single nucleotide.

A “nonsense” mutation is a (point) mutation in a nucleic acid sequence encoding a protein, whereby a codon is changed into a stop codon. This results in a premature stop codon being present in the mRNA and in a truncated protein. A truncated protein may have reduced function or loss of function.

A “missense” or non-synonymous mutation is a (point) mutation in a nucleic acid sequence encoding a protein, whereby a codon is changed to code for a different amino acid. The resulting protein may have reduced function or loss of function.

A “splice-site” mutation is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have reduced function or loss of function.

A “frame-shift” mutation is a mutation in a nucleic acid sequence encoding a protein by which the reading frame of the mRNA is changed, resulting in a different amino acid sequence. The resulting protein may have reduced function or loss of function.

A mutation in a regulatory sequence, e.g. in a promoter of a gene, is a change of one or more nucleotides compared to the wild type sequence, e.g. by replacement, deletion or insertion of one or more nucleotides, leading for example to reduced or no mRNA transcript of the gene being made.

“Silencing” refers to a down-regulation or complete inhibition of gene expression of the target gene or gene family.

A “target gene” in gene silencing approaches is the gene or gene family (or one or more specific alleles of the gene) of which the endogenous gene expression is down-regulated or completely inhibited (silenced) when a chimeric silencing gene (or ‘chimeric RNAi gene’) is expressed and for example produces a silencing RNA transcript (e.g. a dsRNA or hairpin RNA capable of silencing the endogenous target gene expression). In mutagenesis approaches, a target gene is the endogenous gene which is to be mutated, leading to a change in (reduction or loss of) gene expression or a change in (reduction or loss of) function of the encoded protein.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which are joined to form a functional protein, which displays the functionality of the joined domains. A chimeric protein may also be a fusion protein of two or more proteins occurring in nature.

The term “food” is any substance consumed to provide nutritional support for the body. It is usually of plant or animal origin, and contains essential nutrients, such as carbohydrates, fats, proteins, vitamins, or minerals. The substance is ingested by an organism and assimilated by the organism's cells in an effort to produce energy, maintain life, or stimulate growth. The term food includes both substance consumed to provide nutritional support for the human and animal body.

It is understood that comparisons between different plant lines involves growing a number of plants of a line (e.g. at least 5 plants, preferably at least 10, 15 or 20 plants per line) under the same conditions as the plants of one or more control plant lines (preferably wild type plants) and the determination of statistically significant differences between the plant lines when grown under the same environmental conditions.

The “ripening stage” of a tomato fruit can be divided as follows: (1) Mature green stage: surface is completely green; the shade of green may vary from light to dark. (2) Breaker stage: there is a definite break in color from green to tannish-yellow, pink or red on not more than 10% of the surface; (3) Turning stage: 10% to 30% of the surface is not green; in the aggregate, shows a definite change from green to tannish-yellow, pink, red, or a combination thereof. (4) Pink stage: 30% to 60% of the surface is not green; in the aggregate, shows pink or red color. (5) Light red stage (or late orange stage): 60% to 90% of the surface is not green; in the aggregate, shows pinkish-red or red. (6) Red stage (or Red Ripe stage): More than 90% of the surface is not green; in the aggregate, shows red color. It is noted that both normal tomato fruits (i.e. red when ripe) and pink fruits of the invention, have similar ripening stages. The color in the Red stage (6) will however be different: pink in fruits of the invention and red in normal (Wild type) tomato fruits.

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“Sequence identity” and “sequence similarity” can be determined by alignment of two peptide or two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximizing the number of matches and minimises the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62 (Henikoff & Henikoff, 1992, PNAS 89, 10915-10919). Sequence alignments and scores for percentage sequence identity may for example be determined using computer programs, such as EMBOSS as available on the world wide web under ebi.ac.uk/Tools/psa/emboss_needle/). Alternatively sequence similarity or identity may be determined by searching against databases such as FASTA, BLAST, etc., but hits should be retrieved and aligned pairwise to compare sequence identity. Two proteins or two protein domains, or two nucleic acid sequences have “substantial sequence identity” if the percentage sequence identity is at least 80%, 85%, 90%, 95%, 98%, 99% or more (e.g. at least 99.1, 99.2 99.3 99.4, 99.5, 99.6, 99.7, 99.8, 99.9 or more (as determined by Emboss “needle” using default parameters, i.e. gap creation penalty=10, gap extension penalty=0.5, using scoring matrix DNAFULL for nucleic acids an Blosum62 for proteins).

When reference is made to a nucleic acid sequence (e.g. DNA or genomic DNA) having “substantial sequence identity to” a reference sequence or having a sequence identity of at least 80%, e.g. at least 85%, 90%, 95%, 98%, 99%, 99.2%, 99.5%, 99.9% nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

As used herein, the term “plant” includes the whole plant or any parts or derivatives thereof, such as plant organs (e.g., harvested or non-harvested fruits, flowers, leaves, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries, fruits (e.g., harvested tissues or organs, such as harvested tomatoes or parts thereof), flowers, leaves, seeds, tubers, clonally propagated plants, roots, stems, cotyledons, hypocotyls, root tips and the like. Also any developmental stage is included, such as seedlings, immature and mature, etc. As used herein, the term plant includes plant and plant parts comprising one or more of the mutant myb12 alleles and/or myb12 proteins of the invention and/or a mutation in an allele involved in cuticle development, especially in cutin content of the fruit cuticle and/or cuticle layer thickness of the fruits.

In another embodiment, the term plant part refers to plant cells, or plant tissues or plant organs; that comprise one or more of the mutant myb12 alleles and/or myb12 mRNA (cDNA) and/or myb12 protein of the invention and in addition comprise a mutation in an allele involved in cuticle development. In one aspect a plant part can grow into a plant and/or live on photosynthesis (i.e. synthesizing carbohydrate and protein from the inorganic substance, such as water, carbon dioxide and mineral salt). In another aspect, a plant part cannot grow into a plant and/or live on photosynthesis (i.e. synthesizing carbohydrate and protein from the inorganic substance, such as water, carbon dioxide and mineral salt).

A “plant line” or “breeding line” refers to a plant and its progeny. As used herein, the term “inbred line” refers to a plant line which has been repeatedly selfed.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of one locus or gene (or a series of phenotypical characteristics due to this single locus or gene), but which can otherwise differ from one another enormously as regards the other loci or genes.

“F1, F2, etc.” refers to the consecutive related generations following a cross between two parent plants or parent lines. The plants grown from the seeds produced by crossing two plants or lines is called the F1 generation. Selfing the F1 plants results in the F2 generation, etc. “F1 hybrid” plant (or F1 seed) is the generation obtained from crossing two inbred parent lines. An “M1 population” is a plurality of mutagenized seeds/plants of a certain plant line or cultivar. “M2, M3, M4, etc.” refers to the consecutive generations obtained following selfing of a first mutagenized seed/plant (M1).

The term “gloss”, “glossy” or “glossiness” or “brightness” in relation to tomato fruit relates to the level of specular reflection by the surface of the tomato fruit. Gloss is an attribute that causes the surface of a tomato fruit to have a shiny or lustrous appearance. Gloss will increase with the ability of a surface to reflect light without scattering. Hence gloss is often measured by directing a constant power light beam at an angle to the test surface and subsequently by monitoring the amount of reflected light. However, fruit glossiness can also be measured visually by scoring the reflection of light relative to a positive and/or negative control (e.g. such as fruit of a WT cultivated tomato plant). Alternatively, in a photographic picture of a fruit, one can take the number of light saturated pixels as a measure for glossiness (especially when different fruits are measured under the same conditions such as light intensity, angle, surroundings and position of fruit with respect to light source. In tomato (Solanum lycopersicum) fruits the cuticle embedding epidermal cells has a crucial role in tomato fruit brightness, however, no obvious link could be made between cutin load (i.e. fruit cutin content of the cuticle and/or fruit cuticle layer thickness) and fruit brightness (vide supra). Fruit cuticle layer thickness reaches full maturity at Red Ripe stage of tomato fruit development. Consumers often correlate fruit glossiness with fruit quality and it is therefore an important fruit characteristic. Similarly, when a tomato fruit is referred to as dull, it is not glossy. Gloss can be measured visually by comparing the glossiness of two or more objects relative to each other, alternatively a Gloss Meter can be used. See e.g. Example 3. A tomato plant which produces fruits with a statistically “significantly increased glossiness” or “increased glossiness” is herein thus a plant wherein the average glossiness of a number of fruits and a number of plants of that line or variety is (statistically) significantly higher than in the fruits of the control (e.g. homozygous for the wild type CD allele e.g. CD2/CD2 encoding a functional CD2 protein) e.g. as measured by measuring surface reflection of the fruits at e.g. red ripe stage as described above and in the Examples.

The term “allele(s)” means any of one or more alternative forms of a gene at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

The term “locus” (loci plural) means a specific place or places or a site on a chromosome where for example a gene or genetic marker is found. The Myb12 locus is thus the location in the genome where the Myb12 gene is found.

“Wild type allele” (WT or Wt) refers herein to a version of a gene encoding a fully functional protein (wild type protein). Such a sequence encoding a fully functional Myb12 protein is for example the wild type Myb12 cDNA (mRNA) sequence depicted in SEQ ID NO: 4, based on NCBI EU419748 Solanum lycopersicum MYB12 (MYB12) mRNA, complete cds as disclosed on the ncbi.nlm.nih.gov website under/nuccore/171466740 or the wild type Myb12 genomic sequence depicted in SEQ ID NO: 7. The protein sequence encoded by this wild type Myb12 mRNA is depicted in SEQ ID NO: 1. It consists of 338 amino acids. Other fully functional Myb12 protein encoding alleles (i.e. alleles which confer fruit coloring to the same extent i.e. red tomato fruit when the fruit is in ripe stage, as the protein of SEQ ID NO: 1) may exist in other Solanum lycopersicum plants and may comprise substantial sequence identity with SEQ ID NO: 1, i.e. at least about 85%, 90%, 95%, 98%, 99%, 99.2%, 99.3%, 99.4%, 99.5%, 99.6%, 99.7% sequence identity with SEQ ID NO: 1. Such fully functional wild type Myb12 proteins are herein referred to as “variants” of SEQ ID NO: 1. Likewise the nucleotide sequences encoding such fully functional Myb12 proteins are referred to as variants of SEQ ID NO: 4 and SEQ ID NO: 7.

A wild type (WT) sequence encoding a fully functional CD2 (Cutin Deficient 2) protein is for example encoded by the wild type CD2 cDNA (mRNA) sequence depicted in SEQ ID NO: 12, based on NCBI NM_001247728 Solanum lycopersicum CD2 (CD2) mRNA, complete cds at world wide web ncbi.nlm.nih under/nuccore/NM_001247728 (http://www.ncbi.nlm.nib.gov/nuccore/NM_001247728 or by the wild type CD2 genomic sequence depicted in SEQ ID NO: 14. The wild type (functional) protein sequence encoded by this wild type CD2 mRNA is depicted in SEQ ID NO: 10. It consists of 821 amino acids. Other fully functional CD2 protein encoding alleles (i.e. alleles which confer cuticle development to the same extent i.e. glossy tomato fruit when the fruit is in ripe stage, as the protein of SEQ ID NO: 10) may exist in other Solanum lycopersicum plants and may comprise substantial sequence identity with SEQ ID NO: 10, i.e. at least about 85%, 90%, 95%, 98%, 99%, 99.2%, 99.3%, 99.4%, 99.5%, 99.6%, 99.7% sequence identity with SEQ ID NO: 10. Such fully functional wild type CD2 proteins are herein referred to as “variants” of SEQ ID NO: 10. Likewise the nucleotide sequences encoding such fully functional CD2 proteins are referred to as variants of SEQ ID NO: 12 and SEQ ID NO: 14 and may comprise substantial sequence identity with SEQ ID NO: 12 or 14, i.e. at least about 70%, 75%, 85%, 90%, 95%, 98%, 99%, 99.2%, 99.3%, 99.4%, 99.5%, 99.6%, 99.7% sequence identity with SEQ ID NO: 12 or 14.

The following mutant myb12 alleles are exemplary of the myb12 mutants having a less colored epidermis of the tomato fruit at the late orange and/or red stages of fruit development and/or having pink tomato fruit, when in homozygous form, compared to Solanum lycopersicum being homozygous for the wild type Myb12 allele described in the present invention.

It is noted that nucleotide sequences referred to herein (SEQ ID NO: 4-6) are cDNA, i.e. coding DNA sequences, encoding the proteins of SEQ ID NO: 1-3. Counting A in the ATG of the START CODON as nucleotide position 1, SEQ ID's NO: 4-6 have 1017 nucleotides including the TAG STOP-codon. Obviously, when reference is made to these cDNA nucleotide sequences, it is understood that the cDNA is the coding region of the corresponding Solanum lycopersicum genomic myb12 sequence, which, however, additionally contains introns and therefore the nucleotides have different numbering. Thus, when reference is made to a tomato plant comprising an myb12 sequence according to e.g. any one of SEQ ID NO: 4-6, it is, therefore, understood that the tomato plant comprising the genomic myb12 sequence which comprises the coding DNA (cDNA), from which the mRNA of SEQ ID NO: 4-6 is transcribed (and which is in turn translated into protein). The mRNA has the same nucleotide sequence as the cDNA, except that Thymine (T) is Uracil (U) in the mRNA.

Further, when reference is made to a tomato plant comprising a nucleotide sequence encoding a protein according to the invention (i.e. a mutant myb12 protein of SEQ ID No: 2, or 3), this encompasses different nucleotide sequences, due to the degeneracy of the genetic code. In one embodiment the plant comprises the genomic Myb12 sequence depicted in SEQ ID NO:7 or a genomic Myb12 sequence substantially identical thereto (e.g. having at least about 70%, 75%, 80%, 85%, 90%, 95%, 98%, 99%, 99.2%, 99.3%, 99.4%, 99.5%, 99.6%, 99.7% sequence identity with SEQ ID NO: 7), but with one or more mutations in said sequence, especially in the coding sequences of exons of said genomic sequence (coding sequence of exon 1 ranges from nucleotide 1 to 134; exon 2 ranges from nucleotide 225 to 353, exon 3 ranges from nucleotide 1791 to 2140 and exon 4 ranges from nucleotide 2734 to 3137; counting A in the ATG of the START CODON as nucleotide position 1), encoding a mutant myb12 protein causing less-colored and/or colorless epidermis of the tomato fruit. In one embodiment said genomic sequence encodes the mutant myb12 protein of SEQ ID No: 2 or of SEQ ID NO: 3.

One exemplary mutant myb12 allele (mutant 2961; present in seed deposit NCIMB42087 and NCIMB42268) conferring, when in homozygous form, pink tomato fruit and/or less colored epidermis and/or colorless epidermis identified according to the present invention, comprises a mutation resulting in a truncated protein of 60 amino acid residues during translation, whereas the wild type protein has 338 amino acid residues (see SEQ ID NO: 1). The truncated protein sequence of mutant 2961 is depicted in SEQ ID NO: 2. The truncation is due to a change from thymine (T) to an adenine (A) at nucleotide 182 of SEQ ID NO: 4 counting A in the ATG of the START CODON as nucleotide position 1. This T182A mutation in mutant 2961 results in a change from a codon for leucine (i.e. Leu or L) (TTG) to a STOP-codon (TAG). This corresponds to a thymine (T) to an adenine (A) mutation in the genomic DNA at position 1305 of SEQ ID NO: 7. The mutant cDNA is depicted in SEQ ID NO: 5.

Another exemplary mutant myb12 allele (mutant 5505; present in seed deposit NCIMB42088) conferring, when in homozygous form, pink tomato fruit and/or less colored epidermis and/or colorless epidermis identified according to the present invention, comprises a mutation resulting in a change from glycine (Gly or G) to Arginine (Arg or R) at amino acid 50 in the encoded protein (SEQ ID NO: 3) i.e. a G50R mutation. The protein sequence of mutant 5505 is depicted in SEQ ID NO: 3. The amino acid substitution is due to a guanine (G) to cytosine (C) mutation at nucleotide 148 of SEQ ID NO: 4, counting A in the ATG of the START CODON as nucleotide position 1 (i.e. a G148C mutation). This corresponds to a guanine to cytosine mutation in the genomic DNA at position 1271 of SEQ ID NO: 7. The mutant cDNA is depicted in SEQ ID NO: 6.

The following mutant cd2 alleles are exemplary of the cd2 mutants having glossy tomato fruits (e.g. at the late orange and/or red stages of fruit development) i.e. fruits with significantly increased glossiness, when the mutant cd2 allele is in homozygous form, compared to Solanum lycopersicum being homozygous for the wild type CD2 allele described in the present invention. Also when the mutant cd2 allele is in heterozygous form the fruits have significantly increased glossiness compared to fruits of tomato plants homozygous for the wild type CD2 allele (encoding a wild type CD2 protein), i.e. lacking a mutant cd2 allele (encoding a mutant cd2 protein).

Therefore in one aspect a cultivated plant of the species Solanum lycopersicum producing pink glossy fruits is provided comprising a myb12 allele comprising one or more mutations in homozygous form or comprising the y (yellow) gene in homozygous form and comprising a Cuticle Deficiency (cd) allele comprising one or more mutations in homozygous or in heterozygous form, such as the mutant cd2 alleles described below, said mutant cd-allele resulting in an (statistically significant) increased glossiness of the fruits compared to fruits of plants lacking said mutant cd-allele, such as plants lacking the mutant cd2 allele. It is noted that the tomato plants of the invention preferably comprise mutations in alleles of only one of the cd genes selected from cd1, cd2 and cd3, even though the presence of mutant alleles of multiple different CD genes is possible, as the cd1, cd2 and cd3 genes are single recessive genes located on different chromosomes (cd2 was mapped to Chromosome 1, cd3 to chromosome 8 and cd1 to chromosome 11 by Isaacson et al. 2009).

It is noted that nucleotide sequences referred to herein (SEQ ID NO: 12, 13, and 16) are cDNA, i.e. coding DNA sequences, encoding the proteins of SEQ ID NO: 10 (wilt type CD2 protein), SEQ ID NO: 11 (mutant cd2 protein having a G736V, i.e. Glycine (G or Gly) to Valine (V or Val), amino acid change causing glossiness), and SEQ ID NO: 15 (mutant cd2 protein having a Q708H, i.e. Glutamine (Q or Gln) to histidine (H or His), and a D737N (i.e. Aspartic Acid (D or Asp) to Asparagine (N or Asn) amino acid change causing glossiness), respectively. Counting A in the ATG of the START CODON as nucleotide position 1, SEQ ID's NO: 12, 13, and 16 have 2466 nucleotides including the TAA STOP-codon. Obviously, when reference is made to these cDNA nucleotide sequences, it is understood that the cDNA is the coding region of the corresponding Solanum lycopersicum genomic cd2 sequence, which, however, additionally contains introns and therefore the nucleotides have different numbering. Thus, when reference is made to a tomato plant comprising an cd2 sequence according to e.g. any one of SEQ ID NO: 12, 13, or 16, it is, therefore, understood that the tomato plant comprising the genomic cd2 sequence which comprises the coding DNA (cDNA), from which the mRNA of SEQ ID NO: 12, 13, and 16, respectively is transcribed (and which is in turn translated into protein). The mRNA has the same nucleotide sequence as the cDNA, except that Thymine (T) is Uracil (U) in the mRNA.

Further, when reference is made to a tomato plant comprising a nucleotide sequence encoding a protein (involved in cuticle development, especially in cutin production) according to the invention (i.e. a mutant cd2 protein of SEQ ID No: 11, or of SEQ ID NO: 15), this encompasses different nucleotide sequences, due to the degeneracy of the genetic code. In one embodiment the plant comprises the genomic CD2 sequence depicted in SEQ ID NO: 14 or a genomic CD2 sequence substantially identical thereto (e.g. having at least about 70%, 75%, 80%, 85%, 90%, 95%, 98%, 99%, 99.2%, 99.3%, 99.4%, 99.5%, 99.6%, 99.7%, 99.8%, or 99.9% sequence identity with SEQ ID NO: 14), but with one or more mutations in said sequence, especially in the coding sequences of exons of said genomic sequence (the coding sequence in exon 1 ranges from nucleotide 1 to (and including) 204; exon 2 ranges from nucleotide 282 to 502, exon 3 ranges from nucleotide 600 to 715, exon 4 ranges from nucleotide 809 to nucleotide 1494, exon 5 ranges from nucleotide 1617 to nucleotide 1717, exon 6 ranges from nucleotide 1819 to nucleotide 2032, exon 7 ranges from nucleotide 3417 to nucleotide 3591, exon 8 ranges from nucleotide 3718 to nucleotide 4086 and exon 9 ranges from nucleotide 4542 to 4921; counting A in the ATG of the START CODON as nucleotide position 1), encoding a mutant cd2 protein causing glossy tomato fruit as of the Mature Green stage (e.g, Breaker, Orange, or Red Ripe stage) of the tomato fruit development. In one embodiment said genomic sequence encodes the mutant protein of SEQ ID No: 11. In another embodiment the plant comprises the genomic CD2 sequence depicted in SEQ ID NO: 14 comprising a Guanine (G) to Thymine (T) mutation at nucleotide 7171 of SEQ ID NO: 14 (which results in an amino acid change G736V as depicted in SEQ ID NO: 11), or a genomic CD2 sequence substantially identical thereto.

One exemplary mutant cd2 allele (as present in mutant 8.17 and in mutant 26428_001) conferring glossy tomato fruit identified according to the present invention, comprises a mutation resulting in a change from Glycine (Gly or G) to Valine (Val or V) at amino acid 736 in the encoded protein (SEQ ID NO: 11) i.e. a G736V mutation. The cd2 protein sequence of mutant 8.17 (and mutant 26428_001) is depicted in SEQ ID NO: 11. The amino acid substitution is due to a guanine (G) to thymine (T) mutation at nucleotide 2207 of SEQ ID NO: 12, counting A in the ATG of the START CODON as nucleotide position 1 (i.e. a G2207T mutation). The mutant cDNA is depicted in SEQ ID NO: 13. The G2207T mutation in the cDNA of SEQ ID NO: 12 corresponds with the G7171T mutation in the genomic DNA of SEQ ID NO: 14.

Thus in one aspect a tomato plant of the invention comprises a mutant CD2 allele in homozygous or heterozygous form, whereby fruits have increased glossiness compared to the plant lacking the mutant cd2 allele and whereby the mutant cd2 allele encodes a CD2 protein of SEQ ID NO: 10 (or a variant of SEQ ID NO: 10 comprising at least 85%, 90%, 95% or more sequence identity to SEQ ID NO: 10) which comprises a Valine at amino acid 736 (or at an equivalent position in the variant).

One other exemplary mutant cd2 allele (mutant “cd2” as present in Isaacson et al (Isaacson et al, 2009, The plant Journal 60, 363-377); sequence data from this article for CD2 can be found in the GenBank/EMBL data libraries under accession number GQ222185) conferring glossy tomato fruit identified according to the present invention, comprises a mutation resulting in a change from aspartic acid (Asp or D) to asparagine (Asn or N), at amino acid 737 in the encoded protein (SEQ ID NO: 15) i.e. a D737N mutation; and additionally a mutation resulting in a change from glutamine (Gln or Q) to histidine (His or H), at amino acid 708 in the encoded protein (SEQ ID NO: 15) i.e. a Q708H mutation. This “Isaacson 2009” cd2 protein sequence is depicted in SEQ ID NO: 15. Note that in the Results section, Isaacson et al describe their cd2 mutant comprising only a G736R mutation (page 372, left hand column of Isaacson et al, 2009, The plant Journal 60, 363-377). A plant line comprising the mutant cd2 allele described by Isaacson 2009 is available in the ‘Genes that make tomatoes’ collection from Cornell University (line e4393m2). Alternatively, a plant comprising an allele with one or both of the amino acid substitutions may be generated de novo by induced mutagenesis.

Thus in another aspect a tomato plant of the invention comprises a mutant cd2 allele in homozygous or heterozygous form, whereby fruits have increased glossiness compared to the plant lacking the mutant cd2 allele and whereby the mutant cd2 allele encodes a CD2 protein of SEQ ID NO: 10 (or a variant of SEQ ID NO: 10 comprising at least 85%, 90%, 95% or more sequence identity to SEQ ID NO: 10) which comprises a Asparagine at amino acid 737 (or at an equivalent position in the variant) and/or which comprises a Histidine at amino acid 708 (or at an equivalent position in the variant).

Other exemplary mutant Cutin Deficiency (or Cutin Deficient) alleles are cd1 and cd3 as disclosed in Isaacson et al, 2009 (The plant Journal 60, 363-377). Plant lines comprising the mutant cd1 allele (line e4247 ml) or the mutant cd3 allele (line n3056 ml) are available in the ‘Genes that make tomatoes' collection from Cornell University.

Other glossy plants as disclosed in Petit et al (Plant Physiology 2014 vol 164 pp 888-906) such as mutants P23C10, P32H5, P8A12, P5E1, P4B6, P30B6, P6A2, P3H6, P4E2, P15C12, P30A12, P18H8, P17F12, P23F12, P11H2, and P26E8 (as listed in Petit et al, vide supra, Table 1 under Glossy plant).

“Mutant allele” refers herein to an allele comprising one or more mutations in the coding sequence (mRNA, cDNA or genomic sequence) compared to the wild type allele. Such mutation(s) (e.g. insertion, inversion, deletion and/or replacement of one or more nucleotide(s)) may lead to the encoded protein having reduced in vitro and/or in vivo functionality (reduced function) or no in vitro and/or in vivo functionality (loss-of-function), e.g. due to the protein e.g. being truncated or having an amino acid sequence wherein one or more amino acids are deleted, inserted or replaced. Such changes may lead to the protein having a different 3D conformation, being targeted to a different sub-cellular compartment, having a modified catalytic domain, having a modified binding activity to nucleic acids or proteins, etc.

“Wild type plant” and “wild type fruits” or “normal ripening” plants/fruits refers herein to a tomato plant comprising two copies of a wild type (WT or Wt) Myb12 allele (Myb12/Myb12) encoding a fully functional Myb12 protein (e.g. in contrast to “mutant plants”, comprising a mutant myb12 allele in homozygous form). “Wild type fruit glossiness” or “normal fruit glossiness” or “normal glossiness” refers herein to a tomato plant comprising a normal cuticle layer e.g. comprising two copies of the wild type (WT or Wt) CD alleles of the CD1, CD2 and/or CD3 genes encoding functional CD proteins, i.e. CD1/CD1, CD2/CD2 and CD3/CD3; especially plants comprising a wild type CD2 allele (CD2/CD2) encoding a fully functional CD2 protein (e.g. in contrast to “mutant plants”, comprising a mutant cd2 allele in homozygous form). Such plants are for example suitable controls in phenotypic assays. Preferably wild type and/or mutant plants are “cultivated tomato plants”. For example the cultivar Moneymaker is a wild type plant, as is cultivar Ailsa Craig, cultivar Tapa, M82 and many others which are homozygous for the wild CD1, CD2 and CD3 alleles (encoding fully functional CD proteins).

“Tomato plants” or “cultivated tomato plants” are plants of the Solanum lycopersicum, i.e. varieties, breeding lines or cultivars of the species Solanum lycopersicum, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of a species. The so-called heirloom varieties or cultivars, i.e. open pollinated varieties or cultivars commonly grown during earlier periods in human history and often adapted to specific geographic regions, are in one aspect of the invention encompassed herein as cultivated tomato plants.

Wild relatives of tomato include S. arcanum, S. chmielewskii, S. neorickii (=L. parviflorum), S. cheesmaniae, S. galapagense, S. pimpinellifolium, S. chilense, S. corneliomulleri, S. habrochaites (=L. hirsutum), S. huaylasense, S. sisymbriifolium, S. peruvianum, S. hirsutum or S. pennellii.

“Average” or “mean” refers herein to the arithmetic mean and both terms are used interchangeably. The term “average” or “mean” thus refers to the arithmetic mean of several measurements. The skilled person understands that the phenotype of a plant line or variety depends to some extent on growing conditions and that, therefore, arithmetic means of at least 5, 10, 15, 20, 30 or more plants (or plant parts) are measured, preferably in randomized experimental designs with several replicates and suitable control plants grown under the same conditions in the same experiment. “Statistically significant” or “statistically significantly” different or “significantly” different refers to a characteristic of a plant line or variety that, when compared to a suitable control or comparison show a statistically significant difference in that characteristic from the (mean of the) control or comparison (e.g. the p-value is less than 0.05, p<0.05, using ANOVA).

Colour and color are used interchangeably.

The terms “tomato plant producing fruits with certain phenotypic traits” and “tomato plant capable of producing fruits with certain phenotypic traits” are used interchangeably within this document and refer to a (cultivated) tomato plant (Solanum lycopersicum) that is capable of producing fruits. The phenotypic traits that are specified are only visible on the fruits and not necessarily on the plant itself.

### BRIEF DESCRIPTION OF THE SEQUENCE LISTING

SEQ ID NO: 1 shows the Solanum lycopersicum wild type, fully functional, MYB12 protein sequence as derived from the mRNA based on NCBI EU419748 Solanum lycopersicum MYB12 (MYB12) mRNA, complete cds http://www.ncbi.nlm.nih.gov/nuccore/171466740.

SEQ ID NO: 2 shows the Solanum lycopersicum mutant 2961 myb12 protein sequence.

SEQ ID NO: 3 shows the Solanum lycopersicum mutant 5505 myb12 protein sequence.

SEQ ID NO: 4 shows the Solanum lycopersicum wild type Myb12 cDNA based on NCBI EU419748 Solanum lycopersicum MYB12 (MYB12) mRNA, complete cds http://www.ncbi.nlm.nih.gov/nuccore/171466740.

SEQ ID NO: 5 shows the Solanum lycopersicum mutant 2961 myb12 cDNA sequence.

SEQ ID NO: 6 shows the Solanum lycopersicum mutant 5505 myb12 cDNA sequence.

SEQ ID NO: 7 shows the Solanum lycopersicum wild type Myb12 genomic DNA of the same source as under SEQ ID NO: 1 and 4.

SEQ ID NO: 8 shows the mutant 5058 myb12 protein sequence, which does not affect fruit epidermis color.

SEQ ID NO: 9 shows mutant 6899 myb12 protein sequence, which does not affect fruit epidermis color.

SEQ ID NO: 10 shows the Solanum lycopersicum wild type, fully functional, CD2 protein sequence as derived from the mRNA based on NCBI NM_001247728 Solanum lycopersicum CD2 (CD2) mRNA, complete cds at world wide web ncbi.nlm.nih under/nuccore/NM_001247728 (http://www.ncbi.nlm.nih.gov/nuccore/NM_001247728).

SEQ ID NO: 11 shows the Solanum lycopersicum mutant 26428_001 cd2 protein sequence.

SEQ ID NO: 12 shows the Solanum lycopersicum wild type Cd2 cDNA based on NCBI NM_001247728 Solanum lycopersicum CD2 (CD2) mRNA, complete cds at world wide web ncbi.nlm.nih under/nuccore/NM_001247728 (http://www.ncbi.nlm.nih.gov/nuccore/NM_001247728).

SEQ ID NO: 13 shows the Solanum lycopersicum mutant 26428_001 cd2 cDNA sequence.

SEQ ID NO: 14 shows the Solanum lycopersicum wild type Cd2 genomic DNA of the same source as under SEQ ID NO: 10 and 12. The corresponding mutant 26428_001 cd2 genomic DNA comprises a thymine (T) at position 7171 instead of a guanine (G) as in the wild type sequence, i.e. a G7171T mutation in SEQ ID NO: 14.

SEQ ID NO: 15 shows the Solanum lycopersicum mutant cd2 protein sequence of Isaacson et al (vide supra); Genbank EMBL ACCESSION GQ222185 version GQ222185.1 GI:255529748 http://www.ncbi.nlm.nih.gov/nuccore/gq222185.

SEQ ID NO: 16 shows the Solanum lycopersicum mutant cd2 cDNA sequence based on Isaacson et al (vide supra); Genbank EMBL ACCESSION GQ222185 version GQ222185.1 GI:255529748 http://www.ncbi.nlm.nih.gov/nuccore/gq222185.

### BRIEF DESCRIPTION OF THE FIGURES

FIG. 1: Picture of tomato fruit peel (epidermis) of normal (wild type) and mutant fruit. Wild type fruits have a yellow/orange compound in the epidermis which is absent in mutant 1 (i.e. mutant 2961, homozygous for a myb12 allele encoding the protein of SEQ ID NO: 2), having a colorless peel. Mutant 2 (i.e. mutant 5505) shows a yellow/orange tomato fruit peel when the mutant myb12 allele is present in heterozygous form and a colorless peel when the mutant myb12 allele (encoding the protein of SEQ ID NO: 3) is in homozygous form. In homozygous form, the fruits are predominantly pink; only around the place where the fruit was connected to the plant, the epidermis does contain some yellow/orange compound while on the rest of the fruit, this compound is absent in the epidermis.

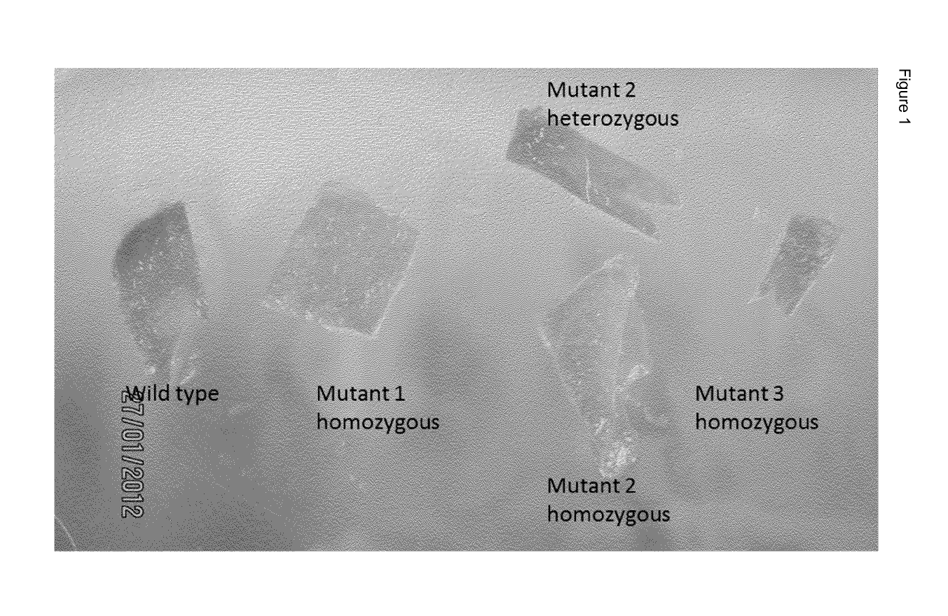

FIG. 2: Table with amino acid sequences of Wt Myb12 protein (SEQ ID NO: 1), mutant 2961 myb12 protein (SEQ ID NO: 2), mutant 5505 myb12 protein (SEQ ID NO: 3) and the Myb12 protein of two other, non-pink, i.e. “normal red” plants, named 5058 (SEQ ID NO: 8) and 6899 (SEQ ID NO: 9).

FIG. 3: Picture of tomato fruits at the Red Ripe stage, of normal and mutant fruit (both for the pink and glossy trait). The top layer shows red tomato fruits (e.g. homozygous to wild type Myb12 allele (Myb12/Myb12) or heterozygous for the wild type Myb12 allele (Myb12/myb12); the lower layer shows pink tomato fruits from plants homozygous for the mutant myb12 allele (myb12/myb12) (as present in mutant 2961); from left to right, the tomato fruits are homozygous for the wild type CD2 allele (CD2/CD2), heterozygous for the mutant type cd2 allele as present in plants of mutant 26428_001 (CD2/cd2), and homozygous for the mutant type cd2 allele as present in plants of mutant 26428_001 (cd/cd2).

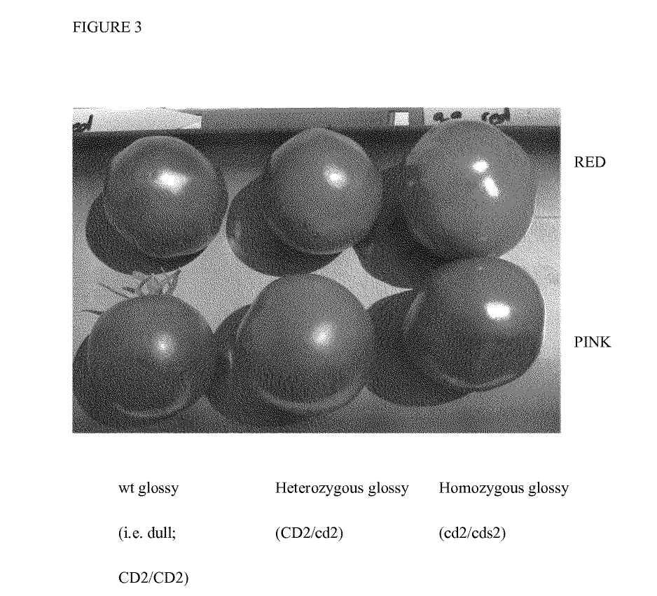

FIG. 4: Wild type and mutant tomato CD2 protein sequence with difference to mutant CD2 protein sequence as submitted by Isaacson et al The Plant Journal, 2009, Vol 60, pp 363-377, under http://www.ncbi.nlm.nih.gov/nuccore/gq222185 and described by Isaacson et al The Plant Journal, 2009, Vol 60, pp 363-377.

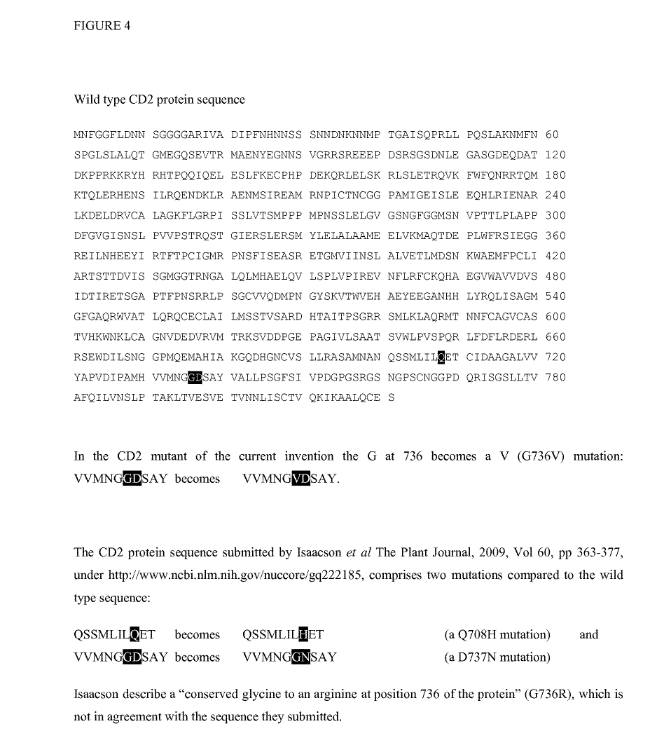

### DETAILED DESCRIPTION OF THE INVENTION

In the broadest sense the invention relates to the combination of alleles causing ‘pink’ fruit color with mutations causing a significant increase in ‘glossiness’ of the fruits and/or significantly increased or decreased amounts of cutin of the fruit cuticles. In one aspect the cuticle thickness is significantly increased and/or decreased, resulting in increased or decreased glossiness. So in one aspect the invention relates to the combination of alleles causing ‘pink’ fruit color with mutations causing a significant increase in ‘glossiness’ of the fruits and/or significantly increased or decreased amounts of cutin of the fruit cuticles and/or a significantly thicker or thinner fruit cuticle layer, respectively (i.e. thicker cuticle with higher cutin amounts and thinner cuticle with lower cutin amounts). Thus any mutant alleles for pink (myb12 mutants and y gene, described herein) are combined with mutant alleles for glossiness and/or cutin accumulation (especially mutant alleles of CD1, CD2 or CD3 genes, described herein). Even when pink and glossy mutants are described in different aspects herein, it is understood that the genetic combination in the genome of a single plant (line or variety), and in the cells of such a single plant (line or variety), is referred to, resulting in pink glossy fruits. Also parent lines are encompassed herein (e.g. inbreds) suitable for producing F1 hybrids which produce pink and glossy fruits. The parent line may be heterozygous for mutant myb12 allele or the y gene, as long as the F1 hybrid is homozygous for myb12 or the y gene to express the pink color.

Thus, in one aspect a cultivated plant of the species Solanum lycopersicum is provided producing pink glossy fruits, comprising a myb12 allele comprising one or more mutations or comprising the y (yellow) gene in homozygous form and comprising a Cuticle Deficiency (cd) allele comprising one or more mutations in homozygous or heterozygous form, said mutant cd-allele resulting in an increased glossiness of the fruits compared to fruits of plants lacking said mutant cd-allele.

The invention discloses a cultivated plant of the species Solanum lycopersicum (capable of) producing pink glossy fruits, comprising a myb12 allele comprising one or more mutations, and comprising a mutation in an allele involved in cuticle development, especially in a CD gene selected from CD1, CD2 and CD3, said mutation resulting in an increased or decreased accumulation of cutin at Red Ripe (RR) stage of at least 15% compared to a wild type plant, i.e. a plant lacking a mutation in an allele involved in cuticle development; and/or in one aspect the cuticle layer is at least a 15% thicker or at least 15% thinner, respectively; and/or said mutation resulting in a significantly increased fruit glossiness compared to the wild type plant, i.e. comprising i.e. a plant lacking a mutation in an allele involved in cuticle development (i.e. having fully functional alleles involved in cuticle development, such as fully functional CD genes).

In one aspect the plant of the invention capable of producing pink glossy fruits comprises a myb12 allele having one or more mutations (referred herein also to as “mutant myb12 allele”), said mutations resulting in production of a mutant myb12 protein. In another aspect the myb12 allele having one or more mutations has a mutation selected from the group consisting of mutation in coding region, mutation in non-coding region, mutation in a promotor of the myb12 allele, and in a gene regulating the expression of the myb12 allele. In another aspect, the mutation resulting in production of a mutant myb12 protein is the mutation causing the y mutant phenotype i.e. due to a mutation in a regulatory gene as known in the art (e.g. Adato et al 2009 PLoS Genetics, Vol 5 issue 12, e1000777).

In still another aspect, the mutation resulting in production of a mutant myb12 protein or lower myb12 protein levels in the plant of the invention capable of producing pink glossy fruits, is a mutation in the myb12 allele. It is understood that said lower myb12 protein level is compared with a plant lacking said myb12 allele comprising one or more mutations.

mutation resulting in production of a mutant myb12 protein. Preferably, the plant lacking said mutation has the same genetic make-up as the plant of the invention except for the myb12 allele. In yet another aspect this mutation in the myb12 allele results in the production of a mutant myb12 protein.

In another aspect the plant of the invention capable of producing glossy pink fruits comprising a myb12 allele comprising one or more mutations resulting in the production of a mutant myb12 protein, wherein said mutant myb12 protein has a G50R amino acid substitution in SEQ ID NO: 1 (i.e. relative to the wild type protein of SEQ ID NO: 1), or in (functional) variants of SEQ ID NO:1 (i.e. relative to functional variants of the wild type protein of SEQ ID NO:1), said variants having at least about 85% amino acid sequence identity to SEQ ID NO: 1; or having at least about 90%, 93%, 95%, 96%, 97%, 98%, or 99% amino acid sequence identity to SEQ ID NO: 1, and in addition to the sequence identity to SEQ ID NO: 1 having said G50R amino acid substitution; or wherein said mutant myb12 protein comprises a deletion of the amino acids 61 to 338 in SEQ ID NO: 1 (i.e. relative to the wild type protein of SEQ ID NO: 1), or in (functional) variants thereof (i.e. relative to functional variants of the wild type protein of SEQ ID NO:1), said variants having at least about 85% amino acid sequence identity to SEQ ID NO: 1; or having at least about 90%, 93%, 95%, 96%, 97%, 98%, or 99% amino acid sequence identity to SEQ ID NO:1, alternatively said variants having at least 95% (e.g. 96%, 97%, 98%, 99%) amino acid sequence identity to amino acids 1 to 60 of SEQ ID NO: 1. In other words, the mutant myb12 protein comprises amino acids 1 to 60 of SEQ ID NO: 1, or amino acids 1 to 60 of a functional variant of SEQ ID NO: 1, said variant having at least about 85%, 90%, 93%, 95%, 96%, 97%, 98%, or 99% amino acid sequence identity to SEQ ID NO: 1. When reference is made to mutant myb12 protein having a G50R amino acid substitution in (functional) variants of SEQ ID NO: 1, such (functional) variants of SEQ ID NO:1 have in addition to the G50R substitution at least about 85% amino acid sequence identity to SEQ ID NO: 1; or have at least about 90%, 93%, 95%, 96%, 97%, 98%, or 99% amino acid sequence identity to SEQ ID NO: 1. In other words, the G50R substitution must be present in the variant of SEQ ID NO: 1, thereby rendering the variant to be a reduced function myb12 protein according to the invention.

It is understood that whenever reference is made to a (functional) variants of SEQ ID NO:1 having at least about 85% amino acid sequence identity to SEQ ID NO: 1 (or any other percentage sequence identity), and having said G50R amino acid substitution, the position of the G to R amino acid substitution may vary a some amino acid positions, e.g. −5, −4, −3, −2, −1, +1, +2, +3, +4, +5 amino acid position due to one or more amino acid deletions or insertions that may occur in the variant of SEQ ID NO: 1. It is further understood that the same rationale applies whenever reference is made to variants of other sequences having a mutation at a specific position.

In another aspect the plant of the invention capable of producing glossy pink fruits comprising a myb12 allele comprising one or more mutations resulting in the production of a lower amount of myb12 protein comprises they (yellow) allele.

Whether a variant of SEQ ID NO: 1 is functional can be tested phenotypically, i.e. by determining if the tomato plant which is homozygous for the allele encoding the variant of SEQ ID NO:1 produces a coloured (yellow-orange) epidermis on tomato fruits at the red-ripe stage of fruit development, in which case it is a functional Myb12 protein.

In one embodiment the invention relates to a cultivated tomato plant of the invention wherein said mutation or mutations result in the fruits of said plant exhibiting a pink appearance at the late orange and/or red stages of fruit development, when the mutant myb12 allele is in homozygous form.

In another embodiment, the invention relates to a cultivated tomato plant capable of producing glossy pink fruits wherein said mutation or mutations result in the fruits of said plant exhibiting a pink appearance at the late orange and/or red stages of fruit development when compared to Solanum lycopersicum being homozygous for the wild type Myb12 allele, when the mutant myb12 allele is in homozygous form or when the y gene is in homozygous form.

In yet another embodiment, the invention relates to a cultivated tomato plant of the invention wherein said mutation or mutations result in the fruits of said plant exhibiting a less colored epidermis than the epidermis of tomato plants homozygous for the wild type Myb12 allele (e.g. encoding the protein of SEQ ID NO: 1), or a colorless epidermis, of the tomato fruit at the late orange and/or red stages of fruit development, when the mutant myb12 allele is in homozygous form.

In an aspect, the invention relates to cultivated tomato plant of the invention (i.e. capable of producing glossy pink fruits) comprising a myb12 allele as found in, and which is derivable from or obtainable from (or derived from or obtained from) seed deposited under accession number NCIMB 42087 or NCIMB 42088 in one or two copies, i.e. in homozygous or heterozygous form. In heterozygous form, the other allele may be a wild type Myb12 allele or another mutant myb12 allele, such as from any one of the other myb12 mutants provided herein, or any other mutant myb12 allele encoding for a loss-of-function myb12 protein or reduced-function myb12 protein as described herein. In heterozygous form, the other allele may, thus, be a reduced-function or a loss-of-function myb12 allele. If both alleles are reduced-function or loss-of-function myb12 alleles, the epidermis will be colorless, or have significantly reduced pigmentation, compared to plants homozygous for the wild type Myb12 allele or compared to plants comprising one copy (heterozygous) for a functional (wild type or variant) Myb12 allele.

In one aspect plants of the invention (i.e. capable of producing glossy pink fruits) are obtainable by crossing a plant of which seeds where deposited under accession number NCIMB 42087 or NCIMB 42088 with another tomato plant; in another aspect, this other plant is a plant producing glossy tomato fruits.

In another aspect plants of the invention (i.e. capable of producing glossy pink fruits) are obtainable from plants of which seeds where deposited under accession number NCIMB 42087 or NCIMB 42088 by crossing a plant grown from the seeds with another tomato plant in another aspect, this other plant is a plant producing glossy tomato fruits.

In another aspect plants of the invention (i.e. capable of producing glossy pink fruits) can be obtained by crossing a plant of which seeds where deposited under accession number NCIMB 42087 or NCIMB 42088 with another tomato plant, in another aspect, this other plant is a plant producing glossy tomato fruits.

The mutant myb12 allele of NCIMB 42087 or of NCIMB 42088, which confers a colorless peel phenotype when in homozygous form, can thus be transferred to any other tomato plant by traditional breeding, to generate pink fruited varieties, when transferring the allele to red-fleshed tomatoes. The mutant alleles can also be transferred to tomato plants producing other flesh-colors, such as yellow, green, orange, etc. There are several genetic loci which determine fruit flesh color (see Sacks and Francis 2001, J. Amer. Soc. Hort. Sci. 126(2): 221-226). However, in one aspect it is combined with tomato plants producing red fruit flesh color, to give the overall pink appearance of the fruit at red-ripe stage.

In yet another aspect plants of the invention (i.e. capable of producing glossy pink fruits) comprise a mutant myb12 allele such as in seeds deposited under accession number NCIMB 42087 or NCIMB 42088.

In still another aspect plants of the invention (i.e. capable of producing glossy pink fruits) are derivable by crossing a plant of which seeds where deposited under accession number NCIMB 42087 or NCIMB 42088 with another tomato plant, in another aspect, this other plant is a plant producing glossy tomato fruits, i.e. comprising a mutant allele of a gene involved in cuticle development, especially comprising a mutant CD-gene selected from the genes CD1, CD2 and CD3. Progeny of the cross can be selected which comprise both a mutant myb12 allele (or the y gene) (as e.g. described above) in homozygous form and which comprise a mutant cd-allele, especially a mutant cd1, cd2 or cd3 allele comprising a mutation in the CD1, CD2 or CD3 protein encoded by the allele, whereby the fruits produced by the plants have a pink and glossy phenotype.

In another aspect plants of the invention (i.e. capable of producing glossy pink fruits) are obtainable from plants of which seeds where deposited under accession number NCIMB 42087 with another tomato plant, in another aspect, this other plant is a plant producing glossy tomato fruits (especially a plant comprising a mutant cd allele in homozygous or heterozygous form, e.g. cd1, cd2 or cd3).

In another aspect plants of the invention (i.e. capable of producing glossy pink fruits) are obtainable from plants of which seeds where deposited under accession number NCIMB 42088 with another tomato plant, in another aspect, this other plant is a plant producing glossy tomato fruits (especially a plant comprising a mutant cd allele in homozygous or heterozygous form, e.g. cd1, cd2 or cd3).

In another aspect plants of the invention (i.e. capable of producing glossy pink fruits) can be derived by crossing a plant of which seeds where deposited under accession number NCIMB 42088 with another tomato plant, in another aspect, this other plant is a plant producing glossy tomato fruits (especially a plant comprising a mutant cd allele in homozygous or heterozygous form, e.g. cd1, cd2 or cd3).

In another aspect plants of the invention (i.e. capable of producing glossy pink fruits) can be derived by crossing a plant of which seeds where deposited under accession number NCIMB 42087 with another tomato plant, in another aspect, this other plant is a plant producing glossy tomato fruits (especially a plant comprising a mutant cd allele in homozygous or heterozygous form, e.g. cd1, cd2 or cd3).

In another aspect, the myb12 allele having one or more mutations is present in homozygous form in the plant of the invention.

In one aspect the myb12 allele having one or more mutations is present in heterozygous form in the plant of the invention.

In one aspect the y gene is in homozygous form.

In one aspect, the invention discloses a cultivated plant of the species Solanum lycopersicum (capable of) producing pink glossy fruits, comprising a myb12 allele comprising one or more mutations in homozygous form (causing a colorless fruit epidermis or an epidermis having significantly reduced pigmentation), and additionally comprising a Cuticle Deficiency (cd) allele comprising one or more mutations in homozygous or heterozygous form, said mutant cd-allele resulting in an increased glossiness of the fruits compared to fruits of plants lacking said mutant cd-allele. In one aspect the mutant cd allele is one of the alleles described elsewhere herein, especially a mutant cd2 allele, such as an allele encoding the protein of SEQ ID NO: 11 or SEQ ID NO: 15. The increased glossiness can be measured by various methods, e.g. visually or quantitatively, for example by measuring reflection of light as described in the examples, using a Gloss Meter. Average fruit glossiness is preferably statistically significantly higher than for fruits of the wild type (lacking the mutant cd-allele/comprising wild type, fully functional CD alleles). Average glossiness is, thus, preferably at least 1.3 times, 1.5 times, 1.7 times, 2 times, 2.5 times, 3.0 times, 3.5 times or more of the average glossiness of the wild type (non-glossy, dull) fruits, such as fruits of varieties M82, Moneymaker, and other varieties.

In one aspect, the invention discloses a cultivated plant of the species Solanum lycopersicum (capable of) producing pink glossy fruits, comprising a myb12 allele comprising one or more mutations in homozygous form (causing a colorless fruit epidermis or an epidermis having significantly reduced pigmentation), and additionally comprising a mutation in an allele involved in cuticle development (such as a mutant allele of a CD gene), said mutation resulting in an increased or decreased accumulation of cutin at Red Ripe (RR) stage of at least 15% (e.g. at least about 20%, 25%, 30%, 35%, 40%, 45%, 50%, 55%, 60%, 65%, 70%, 75%, 80%, 85%, or even at least 90%, 95%, 97%, 98% increase or decrease) compared to a plant lacking the mutation in an allele involved in cuticle development; and/or in another aspect said mutation in an allele involved in cuticle development (such as a mutant allele of a CD gene) results in an at least a 15% thicker or an at least 15% thinner cuticle layer (e.g. at least about 20%, 25%, 30%, 35%, 40%, 45%, 50%, 55%, 60%, 65%, 70%, 75%, 80%, 85%, or even at least 90%, 95%, 97%, 98% thicker or thinner, respectively) compared to a plant lacking the mutation in an allele involved in cuticle development. Thus in one aspect said mutation in an allele involved in cuticle development (such as a mutant allele of a CD gene) results in both a (statistically) significantly increased or amount of average cutin content of the cuticle and a (statistically) significantly increased or decreased average cuticle layer thickness of the fruits at RR stage compared to the fruits of a plant lacking the mutation in an allele involved in cuticle development.

In another aspect, the invention relates to a plant of the invention (i.e. capable of producing glossy pink fruits) wherein the mutation resulting in an increased or decreased accumulation of cutin and/or increased or decreased cuticle layer thickness at Red Ripe (RR) stage of at least 15% (e.g. at least about 20%, 25%, 30%, 35%, 40%, 45%, 50%, 55%, 60%, 65%, 70%, 75%, 80%, 85%, or even at least 90%, 95%, 97%, 98% increase or decrease) is compared to a wild type plant, such as M82 or Moneymaker or TAPA.

In still another aspect the accumulation of cutin and/or cuticle thickness is compared to a plant having the same genetics as the plant of the invention except for the mutation in an allele involved in cuticle development.

In another aspect the invention relates to a plant of the invention i.e. capable of producing glossy pink fruits wherein the amount of cutin and/or the cuticle layer thickness is less than 70% of normal cultivated plants of the species Solanum lycopersicum. In still another aspect the invention relates to a plant of the invention i.e. capable of producing glossy pink fruits wherein the amount of cutin and/or the cuticle layer thickness is less than 90%, 85%, or less than 70% of that of a plant comprising two wild type CD alleles (e.g. CD2/CD2), such as M82 (e.g. normal/wild type plant). For example, the amount of cutin and/or the cuticle layer thickness is less than 65%, e.g. 60%, 55%, 50%, 45%, 40%, 35%, 30%, 25%, 20%, 15%, or even less than 10% or even less than 5% or less than 4% or 3% or 2% of normal cultivated plants of the species Solanum lycopersicum (lacking a mutant cd-allele). In still a further aspect, the amount of cutin and/or the cuticle layer thickness is less than 65%, e.g. 60%, 55%, 50, 45%, 40%, 35%, 30%, 25%, 20%, 15%, or even less than 10% or even less than 5%, less than 4%, 3% or 2% of M82.

In yet another aspect the invention relates to a plant of the invention i.e. capable of producing glossy pink fruits wherein the average amount of cutin is less than 850 μg cm⁻² or less than 700 μg cm⁻², or less than 600 or 500 μg cm⁻² at the Red Ripe (RR) stage. For example the cutin layer thickness is less than 450 μg cm⁻² e.g. less than 400 μg cm⁻², or less than 350 μg cm⁻² or less than 300 μg cm⁻² or less than 250 μg cm⁻² or less than 200 μg cm⁻² or less than 150 μg cm⁻² or even less than 100 μg cm⁻² or less than 50 μg cm⁻² or less than 40 μg cm⁻² or less than 20 μg cm⁻² (at the Red Ripe stage).

The (average) amount of cutin of the fruit cuticles can be measured by measuring cutin monomer levels as described in Isaacson et al. 2009, (supra) page 375 under Cutin monomer and wax analysis.

In yet another aspect the invention relates to a plant of the invention i.e. capable of producing glossy pink fruits wherein the average cuticle layer thickness is less than 11 μm, 10 μm, 8 μm, 6 μm, 4 μm or even less than 2 μm at the Red Ripe (RR). The cuticle layer thickness (or ‘cuticular membrane’) can be measured e.g. by Transmission Electron Microscopy (TEM), Scanning Electron Microspcopy (SEM) or Light Microscopy, as described by Isaacson et al. 2009, (supra) page 374 and 375 under the same titles.

As already mentioned, in one aspect both the average cutin content and the average cuticle layer thickness are affected by the mutant allele, i.e. values for both above are combined in any combination selected from the two lists.

In still another aspect the invention relates to a plant of the invention i.e. capable of producing glossy pink fruits wherein the glossiness level of the fruits at Red Ripe (RR) stage is at least 1.3 times, 1.5 times, 2.0 times, 2.5 times, 3.0 times, 3.5 times as high as the glossiness level of fruits of the same line or wild type plants lacking the mutation in an allele involved in cuticle development (such as a mutant cd1, cd2 or cd3 allele). In one embodiment the glossiness level is compared to a plant of the same line (i.e. having the same genetics) lacking the mutation in an allele involved in cuticle development but producing pink fruits.

In still another aspect the invention relates to a plant of the invention i.e. capable of producing glossy pink fruits wherein the glossiness level of the pink fruits at Red Ripe (RR) stage is at least 1.3 times, 1.5 times, 2.0 times, 2.5 times, 3.0 times, 3.5 times as high as the glossiness level of fruits of the same line or wild type plants lacking the mutation in an allele involved in cuticle development. In one embodiment the glossiness level is compared to a plant of the same line (i.e. having the same genetics) lacking the mutation in an allele involved in cuticle development but producing pink fruits.

Isaacson et al (vide supra) disclose 3 mutant lines (cd1, cd2, and cd3) that have a reduced cuticle layer thickness (see e.g. Discussion line 5 “dramatic reduction in cutin content”) and a highly glossy phenotypes. These mutants were obtained from “Genes that Make Tomatoes” collection (Menda et al, 2004, In silico screening of a saturated mutation library of tomato, Plant Journal 38, pp 861-872). Isaacson mapped the causal mutation to cd2 to tomato chromosome 1. They claim the causal mutation to be a G736R of the protein. This however is not in agreement with the protein sequence they refer to at the end of the paper (i.e. GenBank/EMBL accession number GQ222185). The Genbank protein sequence does not show the G736R mutation, instead it shows two other mutations compared to the wild type protein sequence (as depicted in SEQ ID NO: 10): at position 708 of the wild type cd2 sequence a glutamine (Q or Gln) has been replaced by a histidine (H or His) (i.e. an Q708H mutation); and in addition at position 737 a aspartic acid (D or Asp) has been replaced by an asparagine (N or Asn) (i.e. an D737N mutation).

The present inventors also identified a mutant plant comprising a mutation in the CD2 gene and resulting in significantly glossier fruits than the wild type plants lacking the mutation, however the mutant had a different amino acid substitution than that of Isaacson 2009, namely a G736V amino acid substitution. The G736V mutant was combined with different myb12 mutants (pink mutants), resulting in tomato fruits producing glossy pink fruits.

The invention therefore relates in one aspect to a plant of the invention i.e. capable of producing glossy pink fruits, wherein the mutation in an allele involved in cuticle development is in an allele of the CD1, CD2, or CD3 gene (e.g. as disclosed by Isaacson et al, vide supra, or as described elsewhere herein). As a result of the mutation the encoded CD1, CD2 or CD3 protein comprises one or more amino acid substitutions, insertions or deletions, thereby significantly increasing fruit glossiness and/or significantly increasing or decreasing cutin levels of the fruit cuticle and/or significantly increasing or decreasing cuticle layer thickness; or wherein the mutation in a CD2 allele involved in cuticle development results in a G736V (Glycine to Valine substitution) amino acid substitution in SEQ ID NO: 10 (as shown in SEQ ID NO: 11) or in variants thereof (of SEQ ID NO: 10) having at least 75%, 80%, 85%, 90%, 95% or more amino acid sequence identity to SEQ ID NO: 10 (and which variants have a Valine at amino acid position 736); or wherein the mutation in a CD2 allele involved in cuticle development results in a Q708H (glutamine to histidine) and/or a D737N (aspartic acid to asparagine) amino acid substitution in SEQ ID NO: 10, or in variants of SEQ ID NO: 10 having at least 75%, 80%, 85%, 90%, 95% or more amino acid sequence identity to SEQ ID NO: 10. The amino acid sequence of a SEQ ID NO: 10 having a Q708H and a D737N amino acid substitution is disclosed in SEQ ID NO: 15 and can be derived from plants described by Isaacson et al, 2009 (The plant Journal 60, 363-377), e.g. by crossing line e4393m2e (of the ‘Genes that make tomatoes’ collection from Cornell University) with a wild type tomato plant, lacking the mutant allele. I.e. normal breeding can be used to transfer the mutant cd2 allele to other tomato plants. Alternatively, methods such as TILLING can be used to identify a plant comprising a mutant cd2 allele which is suitable to enhance glossiness of pink fruits (comprising the mutant myb12 allele or y gene).

Similarly, mutant cd1 and cd3 alleles can be obtained from plant lines comprising the mutant cd1 allele (line e4247 ml) or the mutant cd3 allele (line n3056 ml) which are available in the ‘Genes that make tomatoes’ collection from Cornell University. Or alternatively, cd1 and cd2 can be cloned and sequenced, and thereafter mutant alleles (and plants comprising the alleles) which enhance glossiness can be generated de novo using mutagenisis. Also a tomato CD1 sequence is available under GenBank accession AEJ88779.1 (protein) and JF968592 (genomic DNA), so based on this sequence TILLING mutants in the CD1 target gene can be identified which result in enhanced fruit glossiness.

In another aspect, the invention relates to a plant of the invention i.e. capable of producing glossy pink fruits, comprising a mutant cd2 allele wherein the mutation results in the production of (encodes) a cd2 protein comprising a G736V amino acid substitution in SEQ ID NO: 10 or in variants thereof (i.e. of SEQ ID NO: 10) said variants having at least 75% amino acid sequence identity to SEQ ID NO: 10 and having said G736V amino acid substitution. In yet another embodiment said variants have at least 80%, e.g. 85%, 90%, 95%, 96%, 97%, 98%, 99% or at least 99.5% sequence identity to SEQ ID NO: 10 and comprise the 736 Valine and confer enhanced glossiness of the fruits compared to the wild type (functional) variant CD2 protein.

When referring to specific mutations, such as G736V in “variants” of a sequence, it is understood that the amino acid number/position need not be identical, i.e. the Valine of position 736 of SEQ ID NO: 11 may have a different position number in the variant. However the skilled person is easily able to identify whether it is the equivalent amino acid in the variant, e.g. by looking at the stretch of, e.g. 5, amino acids before and after the mutation (e.g. VVMNGVDSAYV) or by aligning the sequences with each other. So when properly aligned pairwise, the skilled person can identify the equivalent amino acid in the variant protein, due to high amino acid sequence identity (at least 80%, e.g. 85%, 90%, 95%, 96%, 97%, 98%, 99% or at least 99.5% identity of the proteins).

In yet another aspect, the invention relates to a plant of the invention i.e. capable of producing glossy pink fruits, comprising a mutant cd2 allele encoding a CD2 protein having a Q708H and/or a D737N amino acid substitutions in SEQ ID NO: 10, or in variants of SEQ ID NO: 10 having at least 75% amino acid sequence identity to SEQ ID NO: 10 and having said Q708H and/or a D737N amino acid substitutions. In yet another embodiment said variants have at least 80%, e.g. 85%, 90%, 95%, 96%, 97%, 98%, 99% or at least 99.5 or 99.9% sequence identity to SEQ ID NO: 10 and confer enhanced glossiness of the fruits compared to the wild type (functional) variant CD2 protein.

In another aspect, the invention relates to a plant of the invention i.e. capable of producing glossy pink fruits, comprising a mutant cd2 allele, wherein the mutant cd2 allele encodes an mRNA according to SEQ ID NO: 13 or variants thereof having 70% nucleic acid sequence identity to SEQ ID NO: 13 and having a thymine at position 2207. In yet another embodiment said variants have at least 75%, 80%, e.g. 85%, 90%, 95%, 96%, 97%, 98%, 99% or at least 99.5 or 99.9% sequence identity to SEQ ID NO: 13 and encode a mutant cd2 protein which confers enhanced glossiness.

In another embodiment the invention relates to a plant of the invention i.e. capable of producing glossy pink fruits, comprising a mutant cd2 allele wherein the mutant cd2 allele encodes an mRNA according to SEQ ID NO: 12 or variants thereof having 70% nucleic acid sequence identity and has G2207T nucleotide substitution. In yet another embodiment said variants have at least 75%, 80%, e.g. 85%, 90%, 95%, 96%, 97%, 98%, 99% or at least 99.5 or 99.9% sequence identity to SEQ ID NO: 12 and encode a mutant cd2 protein which confers enhanced glossiness.

In still another embodiment the invention relates to a plant of the invention i.e. capable of producing glossy pink fruits, wherein the plant comprises a nucleotide sequence encoding a mutant cd2 protein according to SEQ ID No: 11 or SEQ ID NO: 15.

In still another embodiment the invention relates to a plant of the invention i.e. capable of producing glossy pink fruits, wherein the plant comprises a nucleotide sequence encoding a CD2 protein according SEQ ID NO: 10 comprising one or more of the following amino acid substitutions: G736V, D737N and/or Q708H.

In still another embodiment the invention relates to a plant of the invention i.e. capable of producing glossy pink fruits, wherein the plant comprises a genomic cd2 sequence substantially identical to SEQ ID NO: 14, in particular having at least about 70%, 75%, 80%, 85%, 90%, 95%, 98%, 99%, 99.2%, 99.3%, 99.4%, 99.5%, 99.6%, 99.7% sequence identity with SEQ ID NO: 14 and encoding a G736V amino acid substitution in SEQ ID 10. In a further embodiment the genomic cd2 sequence comprises a guanine (g) to thymine (t) (G7171T) mutation at nucleotide position 7171 of SEQ ID NO: 14.

In yet another embodiment the invention relates to a plant of the invention i.e. capable of producing glossy pink fruits, wherein said mutant cd2 allele is in heterozygous form. As it was found that CD2/cd2 plants produced significantly glossier fruits than wild type CD2/CD2 plants, the use of mutant cd alleles in heterozygous form, such as CD2/cd2 (or CD1/cd1; CD3/cd3) is also an embodiment of the invention in combination with a mutant myb12 allele (preferably in homozygous form) or the y gene (preferably in homozygous form), resulting in pink glossy fruits.

In yet another embodiment the invention relates to a plant of the invention i.e. capable of producing glossy pink fruits, wherein said mutant cd2 allele is in homozygous form. As it was found that cd2/cd2 plants produced very glossy fruits, much glossier than fruits of plants comprising the wild type CD2/CD2 allele, the use of mutant cd alleles in homozygous form, such as cd2/cd2 (or cd1/cd1; cd3/cd3) is also an embodiment of the invention in combination with a mutant myb12 allele (preferably in homozygous form) or the y gene (preferably in homozygous form), resulting in pink glossy fruits.

It is noted that pink alleles are in one aspect combined with mutants of only one gene involved in cuticle development selected from the CD1 gene, CD2 gene or CD3 gene, even though stacking of multiple mutant cd genes would be possible as the genes are on different chromosomes. So, either one of the (mutant alleles of the) CD1, CD2 or CD3 gene is combined in a tomato plant with the mutant myb12 alleles or the y gene, while the tomato plant comprises wild type (functional) alleles for the other CD genes (e.g. if a mutant cd2 allele is used in homozygous or heterozygous form, the CD1 and CD3 genes are wild type, functional).

In yet another embodiment the invention relates to a plant of the invention i.e. capable of producing glossy pink fruits, wherein the mutation in the allele involved in cuticle development is a mutation in the mutant cd2 allele as present in seeds deposited under NCIMB 42268. Traditional breeding can be used to combine this allele with a mutant myb12 allele or y gene.

In another embodiment the invention relates to a plant of the invention i.e. capable of producing glossy pink fruits, wherein the mutation in the allele involved in cuticle development is a mutation in the mutant cd2 allele as present in seeds deposited under NCIMB 42269. Traditional breeding can be used to combine this allele with a mutant myb12 allele or y gene.

In still another aspect, the cultivated tomato plant of the invention (i.e. capable of producing glossy pink fruits) is an F1 hybrid. The F1 hybrid preferably comprises two mutant myb12 alleles according to the invention. In a further aspect, the F1 hybrid comprises two mutant cd2 alleles according to the invention. In another aspect, the F1 hybrid comprises two mutant myb12 alleles according to the invention, and one or two mutant cd2 alleles according to the invention. An F1 hybrid is made from two inbred parental lines, which are also an aspect of the invention, as these comprise at least one mutant myb12 allele, or optionally two mutant myb12 alleles (homozygous for myb12). Alternatively, these parental lines comprise at least one mutant myb12 allele and one mutant cd2 allele according to the invention. In one aspect the parental lines comprise two mutant myb12 alleles and two mutant cd2 alleles according to the invention.

The invention also relates to seeds from which a plant according to the invention can be grown.

In another aspect the invention relates to a container comprising seeds from which a plant according to the invention can be grown.

In still another aspect the invention relates to plant parts of a plant of the invention (i.e. capable of producing glossy pink fruits) comprising the myb12 allele comprising the one or more mutations, or comprising the y gene, and additionally comprising a mutation in an allele involved in cuticle development, i.e. comprising a mutant cd allele, such as cd2, which results in significantly enhanced fruit glossiness. As the mutant alleles are combined in the genome of the plant, all vegetative cells of the plant and of plant parts comprise the combination. In addition, some of the reproductive cells (pollen, ovaries) will also retain the combination. So in one aspect, all vegetative cells and plant parts comprising vegetative cells having the mutant alleles as described herein are encompassed, as are reproductive cells which retain the mutant alleles.

In one aspect the invention relates to plant parts of the plant of the invention such as e.g. tomato fruit, seeds, pollen, cells or progeny which comprise the combination of mutant alleles in their genome.

In still another aspect the invention relates to plants and plant parts (e.g. tomato fruit, seeds, pollen, cells or progeny) of a plant of the invention (i.e. capable of producing glossy pink fruits) comprising a myb12 allele having one or more mutations, said myb12 allele being selected from the group consisting of:

a myb12 allele comprising a mutation resulting in production of a mutant myb12 protein, wherein said mutant myb12 protein has a G50R amino acid substitution in SEQ ID NO: 1 or in variants thereof, said variants having at least 85% amino acid sequence identity to SEQ ID NO: 1 (such as e.g. 90%, 95%, 97%, 98%, 99%, 99.5% or 99.8%);
a myb12 allele comprising a mutation resulting in production of a mutant myb12 protein wherein said mutant myb12 protein comprises a deletion of the amino acids 61 to 338 in SEQ ID NO: 1, or in variants thereof, said variants having at least 85% amino acid sequence identity to SEQ ID NO: 1 (such as e.g. 0%, 95%, 97%, 98%, 99%, 99.5% or 99.8%); and the y (yellow) gene;
and wherein said plant or plant parts further comprise a mutation in an allele involved in cuticle development selected from the group consisting of:
a cd2 allele comprising a mutation resulting in the production of a G736V amino acid substitution in SEQ ID NO: 10 or variants thereof having at least 75% amino acid sequence identity to SEQ ID NO: 10 (such as e.g. 80%, 85%, 90%, 95%, 97%, 98%, 99%, 99.5% or 99.8%); and
a cd2 allele comprising a mutation resulting in the production of a Q708H and/or a D737N amino acid substitution in SEQ ID NO: 10 or variants thereof having at least 75% amino acid sequence identity to SEQ ID NO: 10 (such as e.g. 80%, 85%, 90%, 95%, 97%, 98%, 99%, 99.5% or 99.8%).

In yet another aspect the invention relates to plants or plant parts (e.g. tomato fruit, seeds, pollen, cells or progeny) of a plant of the invention (i.e. capable of producing glossy pink fruits) comprising a myb12 allele having one or more mutations resulting in production of a mutant myb12 protein, wherein said mutant myb12 protein has a G50R amino acid substitution in SEQ ID NO: 1 or in variants thereof, said variants having at least 85% amino acid sequence identity to SEQ ID NO: 1, and wherein said plant or plant parts further comprise a mutation in an allele involved in cuticle development selected from the group consisting of:

a cd2 allele comprising a mutation resulting in the production of a G736V amino acid substitution in SEQ ID NO: 10 or variants thereof having at least 75% amino acid sequence identity to SEQ ID NO: 10 (such as e.g. 80%, 85%, 90%, 95%, 97%, 98%, 99%, 99.5% or 99.8%), and
a cd2 allele comprising a mutation resulting in the production of a Q708H and/or a D737N amino acid substitution in SEQ ID NO: 10 or variants thereof having at least 75% amino acid sequence identity to SEQ ID NO: 10 (such as e.g. 80%, 85%, 90%, 95%, 97%, 98%, 99%, 99.5% or 99.8%).

In still another aspect the invention relates to tomato plants and plant parts (e.g. tomato fruit, seeds, pollen, cells or progeny) of a plant of the invention (i.e. capable of producing glossy pink fruits) comprising a myb12 allele having one or more mutations resulting in production of a mutant myb12 protein wherein said mutant myb12 protein comprises a deletion of the amino acids 61 to 338 in SEQ ID NO: 1, or in variants thereof, said variants having at least 75% amino acid sequence identity to SEQ ID NO: 1 (such as e.g. 80%, 85%, 90%, 95%, 97%, 98%, 99%, 99.5% or 99.8%), and wherein said plant parts further comprise a mutation in an allele involved in cuticle development, especially a cd2 allele resulting in the production of a G736V amino acid substitution in SEQ ID NO: 10 or in variants thereof having at least 75% amino acid sequence identity to SEQ ID NO: 10 (such as e.g. 80%, 85%, 90%, 95%, 97%, 98%, 99%, 99.5% or 99.8%); in one aspect a variant having 90% sequence identity to SEQ ID NO: 1 is combined with variants of SEQ ID NO: 10 having at least 75% amino acid sequence identity to SEQ ID NO: 10 (such as e.g. 80%, 85%, 90%, 95%, 97%, 98%, 99%, 99.5% or 99.8%); in another aspect a variant having 95% sequence identity to SEQ ID NO: 1 is combined with variants of SEQ ID NO: 10 having at least 75% amino acid sequence identity to SEQ ID NO: 10 (such as e.g. 80%, 85%, 90%, 95%, 97%, 98%, 99%, 99.5% or 99.8%); in another aspect a variant having 99% sequence identity to SEQ ID NO: 1 is combined with variants of SEQ ID NO: 10 having at least 75% amino acid sequence identity to SEQ ID NO: 10 (such as e.g. 80%, 85%, 90%, 95%, 97%, 98%, 99%, 99.5% or 99.8%). In one aspect the tomato fruit comprising any of the combinations cited above exhibits a pink appearance at the late orange and red stages of fruit development when said myb12 allele is in homozygous form. In one aspect the tomato fruit comprising any of the combination cited above exhibit pink and glossy appearance at the late orange and red stages of fruit development when said myb12 allele is in homozygous form.

In one aspect the invention relates to tomato fruit, seeds, pollen, plant parts, cells or progeny of the plant of the invention comprising the myb12 allele having one or more mutations said mutations resulting in production of a mutant myb12 protein, wherein said mutant myb12 protein has a G50R amino acid substitution in SEQ ID NO: 1 or in a variant thereof, said variant having at least about 85% amino acid sequence identity to SEQ ID NO: 1;

or wherein said mutant myb12 protein comprises a deletion of the amino acids 61 to 338 in SEQ ID NO: 1, or in a variant thereof, said variant having at least 85% amino acid sequence identity to SEQ ID NO: 1.

The presence of one or two copies of a mutant myb12 allele according to the invention in any tomato plant tissue, cells, fruits, pollen, flowers, or other parts of a tomato plant can be determined using standard molecular biology techniques to detect the endogenous allele (genomic DNA), mRNA (cDNA) or protein present. For example, PCR, sequencing, ELISA assays or other techniques may be used.

The presence of one or two copies of a mutant cd2 allele according to the invention in any tomato plant tissue, cells, fruits, pollen, flowers, or other parts of a tomato plant can be determined using standard molecular biology techniques to detect the endogenous allele (genomic DNA), mRNA (cDNA) or protein present. For example, PCR, sequencing, ELISA assays or other techniques may be used.

The invention also relates to tomato fruit of a plant of the invention wherein the tomato fruit exhibit a pink appearance at the late orange and red stages of fruit development and the plant and plant parts are homozygous for a mutant myb12 allele according to the invention. In one aspect the invention relates to tomato fruit of a plant of the invention wherein the tomato fruit exhibit a pink appearance at the late orange and red stages of fruit development compared to Solanum lycopersicum being homozygous for the wild type Myb12 allele, e.g. an allele encoding the protein of SEQ ID NO:1.

The invention also relates to tomato fruit of a plant of the invention wherein the tomato fruit exhibit a glossy pink appearance at the late orange and red stages of fruit development and the plant and plant parts are homozygous for a mutant myb12 allele according to the invention. In one aspect the invention relates to tomato fruit of a plant of the invention wherein the tomato fruit exhibit a glossy pink appearance at the late orange and red stages of fruit development compared to Solanum lycopersicum being homozygous for the wild type Myb12 allele, e.g. an allele encoding the protein of SEQ ID NO:1 and being homozygous for the wild type CD2 allele, e.g. an allele encoding the protein of SEQ ID NO:10.

In still another aspect the invention relates to food or food products comprising or consisting of fruits or parts of said fruit from plants of the invention. Again, the presence of one or two copies of the mutant alleles, such as mutant cd2 or myb12 alleles, of the invention in the food or food products can be detected by standard molecular biology techniques, especially if the food or food product comprises or consists of fresh fruit tissue; depending on the type of tissue it may be difficult to still see the pink and glossy phenotype of fruits of the plant of the invention. In highly processed food products, such as tomato pastes, soups or sauces, it may be difficult to detect the mutant myb12 allele, or fragments thereof (genomic DNA fragments of the myb12 allele), or the mutant myb12 protein, as these may have been destroyed during the processing. In these products, analysis needs to be carried out at an earlier stage.

In another aspect the invention relates to compositions comprising fruit or parts of fruit from plants of the invention. Also a vegetative propagation of plants according to the invention are an aspect encompassed herein. Likewise harvested fruits and fruit parts, either for fresh consumption or for processing or in processed form are encompassed. Fruits may be graded, sized and/or packaged. Fruits may be sliced or diced or further processed.

It is noted that the mutant alleles of the invention can be transferred into any type of cultivated tomato, i.e. producing fruits of various shapes (round, oblong, elongated, pear, etc.) and size (cherry, micro, mini, beefsteak, grape, slicing or globe, plum, pear, etc.). The fruits may be bi-loculate or multi-loculate types. Thus, any such type can produce pink glossy fruits according to the invention. The pink and glossy characteristics can also be combined with the intense fruit phenotype as described in WO2013135726.

The invention also relates to a method for producing a hybrid Solanum lycopersicum plant, said method comprising:

(a) obtaining a first Solanum lycopersicum plant of the invention (e.g. from any one of claims 1-17) or from seed from which a plant of the invention can be grown (e.g. according to claim 18); and
(b) crossing said first Solanum lycopersicum plant with a second Solanum lycopersicum plant to obtain hybrid seeds,
wherein said hybrid Solanum lycopersicum plant grown from said hybrid seeds comprises an myb12 allele having one or more mutations wherein said mutations result in production of a mutant myb12 protein, wherein said mutant myb12 protein has a G50R amino acid substitution in SEQ ID NO: 1 or in variants thereof having at least 85% amino acid sequence identity to SEQ ID NO: 1;
or wherein said mutant myb12 protein comprises a deletion of the amino acids 61 to 338 of SEQ ID NO: 1, or of variants thereof, said variants having at least 85% amino acid sequence identity to SEQ ID NO: 1; or wherein the plant comprises the y (yellow) gene.

In one aspect the plants grown from the seeds produced in step b) also comprise a mutation in an allele involved in cuticle development, such as a cd2 allele comprising a mutation selected from the group consisting of: a mutation resulting in the production of a G736V amino acid substitution in SEQ ID NO: 10 or in variants thereof having at least 75% amino acid sequence identity to SEQ ID NO: 10, and a mutation resulting in the production of a Q708H and/or a D737N amino acid substitution in SEQ ID NO: 10 or in variants thereof having at least 75% amino acid sequence identity to SEQ ID NO: 10.

In one aspect the plants grown from the seeds produced in step b) comprise a mutation in an allele involved in cuticle development, such as a cd2 allele, resulting in the production of a G736V amino acid substitution in SEQ ID NO: 10 or in variants thereof having at least 75% amino acid sequence identity to SEQ ID NO: 10; and in addition these plants have an myb12 allele having one or more mutations wherein said mutations result in production of a mutant myb12 protein, wherein said mutant myb12 protein has a G50R amino acid substitution in SEQ ID NO: 1 or in variants thereof having at least 85% amino acid sequence identity to SEQ ID NO: 1; or wherein said mutant myb12 protein comprises a deletion of the amino acids 61 to 338 of SEQ ID NO: 1, or of variants thereof, said variants having at least 85% amino acid sequence identity to SEQ ID NO: 1; or wherein the plant comprises they (yellow) gene.

In one aspect also the Solanum lycopersicum plant is a plant according to the invention, i.e. comprises at least one mutant cd2 and myb12 allele according to the invention. The resulting F1 hybrid seeds, and plants grown from said seeds, comprise at least one, preferably two mutant cd2 alleles and one, preferably two, mutant myb12 alleles, preferably two identical cd2 alleles and two identical myb12 alleles. The F1 hybrid seeds (and plants grown therefrom) are, thus, in one aspect homozygous for a myb12 allele of the invention and also homozygous for a cd2 allele of the invention.

In still another embodiment the mutant myb12 allele is derived from and/or generated in a cultivated tomato (e.g. a breeding line, variety or heirloom variety) or a wild relative of tomato. Such a human-induced mutation may, for example, be induced using targeted mutagenesis as described in EP1963505. Mutant myb12 alleles generated in wild relatives of tomato are then easily transferred into cultivated tomato by breeding. Similarly, mutant cd2 alleles may be derived from and/or generated in a cultivated tomato (e.g. a breeding line, variety or heirloom variety) or a wild relative of tomato. Such a human-induced mutation may, for example, be induced using targeted mutagenesis as described in EP1963505. Mutant myb12 alleles generated in wild relatives of tomato are then easily transferred into cultivated tomato by breeding. The mutant myb12 allele present in NCIMB 42087 (encoding a myb12 protein of SEQ ID NO: 2) has for example been combined with the mutant cd2 allele as present in NCIMB42269 (encoding a cd2 protein of SEQ ID NO: 11) in a single tomato plant that was deposited under NCIMB 42268.

In another aspect, the invention relates to a tomato plant of the invention having less colored epidermis and/or colorless epidermis and/or pink tomato fruit at the late orange or red stage of fruit development, when compared to wild type (Myb12/Myb12) plants, due to said plants comprising an endogenous myb12 allele, in homozygous form, encoding a loss-of-function myb12 protein or reduced-function myb12 protein, said myb12 protein having substantial sequence identity to SEQ. ID NO: 2 or to SEQ. ID NO: 3 or being 100% identical to the protein of SEQ ID NO: 2 or SEQ ID NO: 3.

In another aspect, the invention relates to a tomato plant of the invention having less colored epidermis and/or colorless epidermis and/or pink tomato fruit at the late orange or red stage (e.g red ripe) of fruit development, when compared to wild type (Myb12/Myb12) plants, due to said plants comprising an endogenous myb12 allele, in homozygous form, encoding a loss-of-function myb12 protein or reduced-function myb12 protein, said myb12 protein having substantial sequence identity to SEQ. ID NO: 2 or to SEQ. ID NO: 3 or being 100% identical to the protein of SEQ ID NO: 2 or SEQ ID NO: 3, wherein the plant further comprises an endogenous cd2 allele, in homozygous or heterozygous form, encoding a CD2 protein of SEQ ID NO: 10 comprising one or more amino acid substitutions selected from: G736V, D737N and Q708H, and produces fruits which are significantly more glossy at the red stage (RR stage) of fruit development when compared to fruits of wild type (CD2/CD2) plants; and/or which fruits comprise a significantly higher or lower amount of cutin at the red stage (RR stage) of fruit development when compared to fruits of wild type (CD2/CD2) plants and/or which fruits comprise a significantly thicker or thinner cuticle layer at the red stage (RR stage) of fruit development when compared to fruits of wild type (CD2/CD2) plants.

In another embodiment the invention relates to an isolated protein having substantial sequence identity to SEQ. ID NO: 2 or to SEQ. ID NO: 3 or 100% sequence identity to SEQ. ID NO: 2 or to SEQ. ID NO: 3. In still a further embodiment, the invention relates to an isolated nucleic acid sequence encoding a protein having substantial sequence identity to SEQ. ID NO: 2 or to SEQ. ID NO: 3 or 100% sequence identity to SEQ. ID NO: 2 or to SEQ. ID NO: 3.

In another embodiment the invention relates to an isolated protein having substantial sequence identity to SEQ. ID NO: 11 or 100% sequence identity to SEQ. ID NO: 11. In still a further embodiment, the invention relates to an isolated nucleic acid sequence encoding a protein having substantial sequence identity to SEQ. ID NO: 11 or 100% sequence identity to SEQ. ID NO: 11.

In an even further embodiment, the invention relates to an isolated nucleic acid sequence, DNA or RNA, having substantial sequence identity to SEQ. ID NO: 5 or to SEQ. ID NO: 6 or having 100% sequence identity to SEQ. ID NO: 5 or to SEQ. ID NO: 6; or to an isolated nucleic acid sequence which is being transcribed into a nucleic acid sequence having substantial sequence identity to SEQ. ID NO: 5 or to SEQ. ID NO: 6 or having 100% sequence identity to SEQ. ID NO: 5 or to SEQ. ID NO: 6.

In an even further embodiment, the invention relates to an isolated nucleic acid sequence, DNA or RNA, having substantial sequence identity to SEQ. ID NO: 13 or having 100% sequence identity to SEQ. ID NO: 13; or to an isolated nucleic acid sequence which is being transcribed into a nucleic acid sequence having substantial sequence identity to SEQ. ID NO: 13 or having 100% sequence identity to SEQ. ID NO: 13.

In still another aspect of the invention tomato plants are provided that have the same or similar epidermis and/or peel color at the red-ripe stage of fruit development as fruits of the tomato plants of the invention, of which representative seeds were deposited by Nunhems B. V. and accepted for deposit on 5 Dec. 2012 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32(1)). Seeds were given the following deposit numbers: NCIMB 42087 (mutant 2961) or NCIMB 42088 (mutant 5505).

In further aspect of the invention tomato plants are provided that have the same or similar glossiness at the red-ripe stage of fruit development, and/or same or similar cutin content, and/or same or similar cuticle layer thickness, as fruits of the tomato plants of the invention, of which representative seeds were deposited by Nunhems B. V under NCIMB 42268 (mutant 8.17) and NCIMB42269 (mutant 26428.001).

In further aspect of the invention tomato plants are provided that have the same or similar glossiness at the red-ripe stage of fruit development, and/or same or similar cutin content, and/or same or similar cuticle layer thickness, as fruits of the F1 tomato plants obtained from crossing a plant grown from seeds deposited under NCIMB 42268 (mutant 8.17; cd2/cd2) or under NCIMB42269 (mutant 26428.001; cd2/cd2) with another tomato plant lacking the cd2 mutant (being wild type for the CD2 gene, CD2/CD2).

In still another aspect of the invention tomato plants are provided that have the same or similar glossiness (and/or cutin content and/or cuticle layer thickness) and the same or similar epidermis and/or peel color at the red-ripe stage of fruit development as fruits of the tomato plants of the invention, of which representative seeds were deposited by Nunhems B. V under NCIMB 42268 (mutant 8.17; myb12/myb12 and cd2/cd2); or as fruits of F1 plants obtained by crossing NCIMB42268 with a wild type tomato plant (Myb12/Myb12 and CD2/CD2).

“Same or similar” means that the characteristic (e.g. color; glossiness; cutin content; cuticle thickness) does not differ in a statistically significant way from the characteristic of the plant described and/or deposited; in other words, there is no statistically significant difference found between the plants (or plant lines or varieties) compared regarding the characteristic (e.g. in a one-way ANOVA the P-value is above 0.05, indicating that there is no significant difference).

According to a further aspect the invention provides a cell culture or a tissue culture of a tomato plant of the invention. The cell culture or tissue culture comprises regenerable cells. Such cells or tissues can be derived from leaves, pollen, embryos, cotyledon, hypocotyls, meristematic cells, roots, root tips, anthers, flowers, seeds or stems of tomato plants according to the invention. In another embodiment, the cell culture or tissue culture does not comprise regenerable cells. In one aspect non-propagating cells of the invention are provided and a cell culture or tissue culture comprising or consisting of non-propagating cells of the invention.

An aspect of the invention is a method of producing a tomato plant of the invention comprising the steps of:
a. obtaining plant material, preferably seeds, of a tomato plant;
b. treating said plant material with a mutagen to create mutagenized plant material, e.g. mutagenized seeds;
c. analyzing said mutagenized plant material, e.g. the mutagenized seeds or progeny thereof obtained by selfing, and
   identifying a plant having at least one mutation in at least one myb12 allele having substantial sequence identity to SEQ ID NO: 7 or in a functional variant thereof; and/or
   identifying a plant having at least one mutation in at least one cd2 allele having substantial sequence identity to SEQ ID NO: 14 or in a functional variant thereof; or
   identifying a plant having at least one mutation in at least one myb12 allele having substantial sequence identity to SEQ ID NO: 7 or in a functional variant thereof and having at least one mutation in at least one cd2 allele having substantial sequence identity to SEQ ID NO: 14 or in a functional variant thereof. The method may optionally further comprise crossing the plant comprising said at least one myb12 allele, or progeny thereof produced by selfing, with said plant comprising said at least one cd2 allele, or with progeny thereof produced by selfing, to obtain a plant comprising both said myb12 and said cd2 allele.

The method may further comprise analyzing the color or glossiness of tomato fruits of the selected plant or progeny of the plant and selecting a plant of which the fruits have pink or pinkish color. In one aspect the mutation is selected from a mutation resulting in an amino acid substitution selected from the group consisting of G50R in SEQ ID NO: 1, or in variants thereof having at least 85% amino acid sequence identity to SEQ ID NO: 1; or wherein said mutant myb12 protein comprises a deletion of the amino acids 61 to 338 of SEQ ID NO: 1, or in variants thereof, said variants having at least 85% amino acid sequence identity to SEQ ID NO: 1.

In a further aspect, the mutation is selected from a mutation causing a change in the cDNA selected from the group consisting of G148C, and T182A in SEQ ID NO: 4.

In this method, in step c) the plant material may be identified which comprises a cd2 allele having at least one mutation in at least one cd2 allele having substantial sequence identity to SEQ ID NO: 14 or in a functional variant thereof. This at least one mutation in one cd2 allele is in one aspect a mutation resulting in a G736V and/or a D737N and/or a Q708H amino acid substitution in SEQ ID NO: 10, or in variants thereof having at least 85% amino acid sequence identity to SEQ ID NO: 10. In yet another embodiment, this at least one mutation in one cd2 allele comprises a G7171T mutation in SEQ ID NO: 14.

In this method, the plant material of step a) is preferably selected from the group consisting of seeds, pollen, plant cells, or plant tissue of a tomato plant line or cultivar. Plant seeds being more preferred. In another aspect, the mutagen used in this method is ethyl methanesulfonate. In step b) and step c) the mutagenized plant material is preferably a mutant population, such as a tomato TILLING population.

Thus, in one aspect a method for producing a tomato plant comprising a mutant myb12 allele is provided comprising the steps of:
a) providing a tomato TILLING population,
b) screening said TILLING population for mutants in the myb12 gene, and
c) selecting from the mutant plants of b) those plants (or progeny of those plants) of which the fruits produce a colorless epidermis or reduced color epidermis compared to wild type (Myb12/Myb12) fruits.

In another aspect a method for producing a tomato plant comprising a mutant cd2 allele is provided comprising the steps of:
i) providing a tomato TILLING population,
ii) screening said TILLING population for mutants in the cd2 gene, and
iii) selecting from the mutant plants of b) those plants (or progeny of those plants) of which the fruits more glossy compared to wild type (CD2/CD2) fruits.

Thus, in another aspect a method for producing a tomato plant according to the invention is provided comprising a mutant myb12 and cd2 allele is provided comprising the steps of crossing a plant selected in step c) (or progeny thereof produced by selfing) with a plant selected in step iii) (or with progeny thereof produced by selfing), and selfing the progeny plants and select progeny that produce pink glossy fruits.

Mutant plants (M1) are preferably selfed one or more times to generate for example M2 populations or preferably M3 or M4 populations for phenotyping in step c). In M2 populations the mutant allele is present in a ratio of 1 (homozygous for mutant allele):2 (heterozygous for mutant allele):1 (homozygous for wild type allele).

In yet a further aspect the invention relates to a method for producing a hybrid Solanum lycopersicum plant, said method comprising:
(a) obtaining a first Solanum lycopersicum plant of the current invention or from a seed from which a plant of the invention can be grown; and
(b) crossing said first Solanum lycopersicum plant with a second Solanum lycopersicum plant to obtain hybrid seeds,
   wherein said hybrid Solanum lycopersicum plant comprises an myb12 allele having one or more mutations wherein said mutations result in production of a mutant myb12 protein which has a G50R amino acid substitution in SEQ ID NO: 1 or in variants thereof, said variants having at least 85% amino acid sequence identity to SEQ ID NO: 1;
   or wherein said mutant myb12 protein comprises a deletion of the amino acids 61 to 338 of SEQ ID NO: 1, or of amino acids 61 to 338 (or amino acids 61 to the end of the protein) in variants of SEQ ID NO: 1, said variants having at least 85% amino acid sequence identity to SEQ ID NO: 1;
   and in addition,
   wherein the plant comprises a mutation in an allele involved in cuticle development, especially a cd2 allele selected from the group consisting of: a cd2 allele comprising a mutation resulting in the production of a G736V amino acid substitution in SEQ ID NO: 10 or variants thereof having at least 75% amino acid sequence identity to SEQ ID NO: 10, and cd2 allele comprising a mutation resulting in the production of a Q708H and/or a D737N amino acid substitution in SEQ ID NO: 10 or variants thereof having at least 75% amino acid sequence identity to SEQ ID NO: 10.

Plants and plant parts (e.g. fruits, cells, etc.) of the invention can be homozygous or heterozygous for the mutant myb12 allele.

Preferably, the plants according to the invention, which comprise one or more mutant myb12 alleles, and which produce a mutant myb12 protein having a G50R amino acid substitution in SEQ ID NO: 1 or in variants thereof having at least 85% amino acid sequence similarity to SEQ ID NO: 1;

or wherein said mutant myb12 protein comprises a deletion of the amino acids 61 to 338 in SEQ ID NO: 1, or in variants thereof, said variants having at least 85% amino acid sequence identity to SEQ ID NO: 1, do not produce fewer fruits than the wild type plants. Thus, fruit number per plant is preferably not reduced.

Other putative MYB12 genes/proteins can be identified in silico, e.g. by identifying nucleic acid or protein sequences in existing nucleic acid or protein database (e.g. GENBANK, SWISSPROT, TrEMBL) and using standard sequence analysis software, such as sequence similarity search tools (BLASTN, BLASTP, BLASTX, TBLAST, FASTA, etc.).

In one embodiment loss-of-function myb12 protein or reduced-function mutant myb12 proteins (including variants or orthologs, such as myb12 proteins of wild tomato relatives) are provided and plants and plant parts comprising one or more myb12 alleles in their genome, which encode loss-of-function myb12 protein or reduced-function mutants, whereby the reduced-function confers pink tomato fruit (in combination of the homozygous myb12 mutant with red fruit flesh) and/or less colored epidermis and/or colorless epidermis, when the mutant allele is in homozygous form, compared to Solanum lycopersicum being homozygous for the wild type Myb12 allele.

In another embodiment mutant proteins are provided having at least about 85% amino acid sequence identity to SEQ ID NO: 1; or having at least about 90%, 93%, 95%, 96%, 97%, 98%, or 99%, or 100% amino acid sequence identity to SEQ ID NO: 1. In another embodiment fragments of such mutant proteins are provided comprising 20, 25, 30, 35, 40, 45, 50, 55, 60, 70, 80, 90, 100, 120, or 150 contiguous amino acids of SEQ ID NO:1 or of the sequences having at least about 90%, 93%, 95%, 96%, 97%, 98%, or 99%, or 100% amino acid sequence identity to SEQ ID NO: 1, including the G50R amino acid substitution in SEQ ID NO: 1. In still another embodiment, nucleic acid sequences encoding such proteins or protein fragments are provided.

In yet another embodiment, the use is provided of the mutant protein or variant thereof, or fragment thereof, as herein defined, in a tomato plant in order to obtain a colorless epidermis of the tomato fruit at the late orange and/or red stages of fruit development. This use is also provided of a nucleic acid encoding such a protein or protein fragment.

Any type of mutation may lead to a reduction in function of the encoded Myb12 protein, e.g. insertion, deletion and/or replacement of one or more nucleotides in the genomic DNA which comprises the cDNA (SEQ ID NO: 4, or variants thereof). However, not all mutations do cause a colorless epidermis as is illustrated in the Examples enclosed herein. In a preferred embodiment an myb12 nucleic acid sequence, encoding a loss-of-function myb12 protein or reduced-function myb12 protein due to one or more mutation(s), is provided, said myb12 protein causing pink tomato fruit (in combination of the homozygous myb12 mutant with red fruit flesh) and/or less colored epidermis and/or colorless epidermis e.g. when compared to Solanum lycopersicum being homozygous for the wild type Myb12 allele.

Similarly, cd nucleic acid sequence, encoding a loss-of-function CD protein or reduced-function CD protein due to one or more mutation(s) (e.g. mutant cd1, cd2 or cd3 protein), is provided, said cd protein causing enhanced glossiness of fruits at red stage, and/or significantly higher or lower cutin levels and/or a significantly thicker or thinner cuticle layer when compared to Solanum lycopersicum being homozygous for the wild type CD allele.

The in vivo loss-of-function myb12 protein or reduced-function of such proteins can be tested as described herein, by determining the effect this mutant allele, in homozygous form, has on the color of the epidermis of late orange or red-ripe stage tomato fruit or by determining the effect of the mutation on the color of the tomato fruits at late orange or red-ripe stage tomato fruit; when the homozygous mutant allele is combined with red fruit flesh the fruit color will become pink. Plants comprising a nucleic acid sequence encoding such mutant loss-of-function myb12 protein or reduced-function proteins and having less-colored epidermis and/or colorless epidermis and/or pink tomato fruit at late orange and/or red ripe stage optionally when compared to Solanum lycopersicum being homozygous for the wild type Myb12 allele can for example be generated de novo using e.g. mutagenesis and identified by TILLING, as known in the art.

The in vivo loss-of-function or reduced function of CD proteins can be tested as described herein, by determining the effect this mutant allele, in homozygous or heterozygous form, has on the glossiness, cutin content and/or cuticle layer thickness at red ripe stage of the fruits.

Also transgenic methods can be used to test in vivo functionality of a mutant myb12 allele or cd allele encoding a mutant myb12 protein or cd protein. A mutant allele can be operably linked to a plant promoter and the chimeric gene can be introduced into a tomato plant by transformation. Regenerated plants (or progeny, e.g. obtained by selfing), can be tested for epidermis color and/or tomato fruit color at late orange and/or red ripe stage. For example a tomato plant comprising a non-functional myb12 allele or cd allele can be transformed to test the functionality of a transgenic myb12 allele or cd allele.

TILLING (Targeting Induced Local Lesions IN Genomes) is a general reverse genetics technique that uses traditional chemical mutagenesis methods to create libraries of mutagenized individuals that are later subjected to high throughput screens for the discovery of mutations. TILLING combines chemical mutagenesis with mutation screens of pooled PCR products, resulting in the isolation of missense and non-sense mutant alleles of the targeted genes. Thus, TILLING uses traditional chemical mutagenesis (e.g. EMS or MNU mutagenesis) or other mutagenesis methods (e.g. radiation such as UV) followed by high-throughput screening for mutations in specific target genes, such as Myb12 according to the invention. S1 nucleases, such as CEL1 or ENDO1, are used to cleave heteroduplexes of mutant and wildtype target DNA and detection of cleavage products using e.g. electrophoresis such as a LI-COR gel analyzer system, see e.g. Henikoff et al. Plant Physiology 2004, 135: 630-636. TILLING has been applied in many plant species, such as tomato. (see http://tilling.ucdavis.edu/index.php/Tomato_Tilling), rice (Till et al. 2007, BMC Plant Biol 7: 19), Arabidopsis (Till et al. 2006, Methods Mol Biol 323: 127-35), Brassica, maize (Till et al. 2004, BMC Plant Biol 4: 12), etc.

In one embodiment of the invention (cDNA or genomic) nucleic acid sequences encoding such mutant myb12 or cd proteins comprise one or more non-sense and/or missense mutations, e.g. transitions (replacement of purine with another purine (A←→G) or pyrimidine with another pyrimidine (C←→T)) or transversions (replacement of purine with pyrimidine, or vice versa (C/T←→A/G). In one embodiment the non-sense and/or missense mutation(s) is/are in the nucleotide sequence encoding any of the Myb12 exons or CD exons, or an essentially similar domain of a variant Myb12 protein or CD protein, i.e. in a domain comprising at least 80%, 90%, 95%, 98%, 99% amino acid sequence identity to amino acids of SEQ ID NO: 1 (Myb12) or SEQ ID NO: 10 (CD2) or to a variant thereof.

In one embodiment an myb12 nucleotide sequence comprising one or more non-sense and/or missense mutations in one of the exon-encoding sequence are provided, as well as a plant comprising such a mutant allele resulting in pink tomato fruit and/or less colored epidermis and/or colorless epidermis optionally when compared to Solanum lycopersicum being homozygous for the wild type Myb12 allele.

In one embodiment an cd2 nucleotide sequence comprising one or more non-sense and/or missense mutations in one of the exon-encoding sequence are provided, as well as a plant comprising such a mutant allele resulting in glossy or significantly glossier tomato fruit when compared to Solanum lycopersicum being homozygous for the wild type CD2 allele.

In a specific embodiment of the invention tomato plants and plant parts (fruits, seeds, etc.) comprising a mutant loss-of-function or reduced-function myb12 allele and/or cd2 allele according to the invention are provided.

Also provided are nucleic acid sequences (genomic DNA, cDNA, RNA) encoding loss-of-function myb12 protein or reduced-function myb12 proteins, such as for example myb12 depicted in SEQ ID NO: 2, or 3 or variants thereof as defined above (including any chimeric or hybrid proteins or mutated proteins or truncated proteins). Due to the degeneracy of the genetic code various nucleic acid sequences may encode the same amino acid sequence. The nucleic acid sequences provided include naturally occurring, artificial or synthetic nucleic acid sequences. A nucleic acid sequence encoding Myb12 is provided for in SEQ ID NO: 4 (NCBI EU419748 Solanum lycopersicum MYB12 (MYB12) mRNA, complete cds http://www.ncbi.nlm.nih.gov/nuccore/171466740).

Also provided are nucleic acid sequences (genomic DNA, cDNA, RNA) encoding loss-of-function cd protein (especially cd2) or reduced-function cd proteins (especially cd2), such as for example cd2 depicted in SEQ ID NO: 11 or 15 or variants thereof as defined above (including any chimeric or hybrid proteins or mutated proteins or truncated proteins).

It is understood that when sequences are depicted as DNA sequences while RNA is referred to, the actual base sequence of the RNA molecule is identical with the difference that thymine (T) is replace by uracil (U). When referring herein to nucleotide sequences (e.g DNA or RNA) italics are used, e.g. myb12 allele, while when referring to proteins, no italics are used, e.g. myb12 protein. Mutants are in small letters (e.g myb12 allele or myb12 protein), while wild type/functional forms start with a capital letter (Myb12 allele or Myb12 protein).

Also provided are nucleic acid sequences (genomic DNA, cDNA, RNA) encoding mutant myb12 proteins, i.e. loss-of-function myb12 protein or reduced function myb12 proteins, as described above, and plants and plant parts comprising such mutant sequences. For example, myb12 nucleic acid sequences comprising one or more non-sense and/or missense mutations in the wild type Myb12 coding sequence, rendering the encoded protein having a loss-of-function or reduced function in vivo. Also sequences with other mutations are provided, such as splice-site mutants, i.e. mutations in the genomic myb12 sequence leading to aberrant splicing of the pre-mRNA, and/or frame-shift mutations, and/or insertions (e.g. transposon insertions) and/or deletions of one or more nucleic acids.

It is clear that many methods can be used to identify, synthesise or isolate variants or fragments of myb12 or cd nucleic acid sequences, such as nucleic acid hybridization, PCR technology, in silico analysis and nucleic acid synthesis, and the like. Variants of SEQ ID NO: 4 (or SEQ ID NO: 10), may either encode wild type, functional Myb12 proteins (or CD2 proteins), or they may encode loss-of-function myb12 protein (or CD2 proteins) or reduced-function mutant alleles of any of these, as for example generated e.g. by mutagenesis by methods such as TILLING, or other methods.

A plant of the invention can be used in a conventional plant breeding scheme to produce more plants with the same characteristics or to introduce the mutated myb12 or cd2 allele into other plant lines or varieties of the same or related plant species.

Also transgenic plants can be made using the mutant myb12 or cd2 nucleotide sequences of the invention using known plant transformation and regeneration techniques in the art. An “elite event” can be selected, which is a transformation event having the chimeric gene (comprising a promoter operably linked to a nucleotide sequence encoding a loss-of-function myb12 or cd protein or reduced-function myb12 or cd protein) inserted in a particular location in the genome, which results in good expression of the desired phenotype.

The plants of the invention as described above are homozygous for the mutant myb12 allele, or heterozygous. Similarly, the plants of the invention described above are homozygous for the mutant cd allele (cd1, cd2 or cd3), e.g. the cd2 allele, or heterozygous. To generate plants comprising the mutant allele in homozygous form, selfing can be used. The mutant myb12 and or cd alleles (e.g. cd2 allele) according to the invention can be transferred to any other tomato plant by traditional breeding techniques, such as crossing, selfing, backcrossing, etc. Thus any type of tomato having comprising at least one mutant myb12 and or cd (e.g. cd2) allele according to the invention can be generated. Any S. lycopersicum may be generated and/or identified having at least one mutant myb12 and or cd (e.g. cd2) allele in its genome and producing a myb12 (or cd protein, e.g. cd2 protein, respectively) having loss-of-function myb12 protein (or cd protein, e.g. cd2 protein) or reduced activity compared to wild type Myb12 (or CD, e.g. CD2) protein. The tomato plant may, thus, be any cultivated tomato, any commercial variety, any breeding line or other, it may be determinate or indeterminate, open pollinated or hybrid, producing fruit flesh of any color, fruits of any shape and size. The mutant allele generated and/or identified in a particular tomato plant, or in a sexually compatible relative of tomato, may be easily transferred into any other tomato plant by breeding (crossing with a plant comprising the mutant allele and then selecting progeny comprising the mutant allele).

The presence or absence of a mutant myb12 allele or cd allele (e.g. cd2 allele) according to the invention in any tomato plant or plant part and/or the inheritance of the allele to progeny plants can be determined phenotypically and/or using molecular tools (e.g. detecting the presence or absence of the myb12 or cd nucleotide sequence or myb12 or cd protein using direct or indirect methods).

The mutant allele is in one embodiment generated or identified in a cultivated plant, but may also be generated and/or identified in a wild plant or non-cultivated plant and then transferred into an cultivated plant using e.g. crossing and selection (optionally using interspecific crosses with e.g. embryo rescue to transfer the mutant allele). Thus, a mutant myb12 allele or cd allele may be generated (human induced mutation using mutagenesis techniques to mutagenize the target myb12 gene or cd gene or variant thereof) and/or identified (spontaneous or natural allelic variation) in Solanum lycopersicum or in other Solanum species include for example wild relatives of tomato, such as S. cheesmanii, S. chilense, S. habrochaites (L. hirsutum), S. chmielewskii, S. lycopersicum×S. peruvianum, S. glandulosum, S. hirsutum, S. minutum, S. parviflorum, S. pennellii, S. peruvianum, S. peruvianum var. humifusum and S. pimpinellifolium, and then transferred into a cultivated Solanum plant, e.g. Solanum lycopersicum by traditional breeding techniques. The term “traditional breeding techniques” encompasses herein crossing, selfing, selection, double haploid production, embryo rescue, protoplast fusion, transfer via bridge species, etc. as known to the breeder, i.e. methods other than genetic modification by which alleles can be transferred.

In another embodiment, the plant comprising the mutant myb12 allele and/or mutant cd allele (e.g. tomato) is crossed with another plant of the same species or of a closely related species, to generate a hybrid plant (hybrid seed) comprising the mutant myb12 allele and/or cd allele. Such a hybrid plant is also an embodiment of the invention.

In one embodiment F1 hybrid tomato seeds (i.e. seeds from which F1 hybrid tomato plants can be grown) are provided, comprising at least one mutant myb12 allele and/or at least one mutant cd allele according to the invention, preferably two myb12 alleles and/or one or two cd allele (e.g. cd2). F1 hybrid seeds, also referred to as hybrid seeds, are seeds harvested from a cross between two inbred tomato parent plants. Such an F1 hybrid may comprise one or two mutant myb12 alleles according to the invention and/or one or two mutant cd alleles according to the invention (e.g. mutant cd2). Such an F1 hybrid comprising two mutant myb12 alleles according to the invention may comprise two copies of the same myb12 allele or two different myb12 alleles according to the invention. Thus, in one embodiment a plant according to the invention is used as a parent plant to produce an F1 hybrid. An F1 hybrid comprising two mutant cd alleles according to the invention may comprise two copies of the same cd allele (e.g. cd2/cd2 both encoding the protein of SEQ ID NO: 11) or two different cd alleles according to the invention (e.g. one cd2 encoding the protein of SEQ ID NO: 11 and one cd2 encoding the protein of SEQ ID NO: 15). Thus, in one embodiment a plant according to the invention is used as a parent plant to produce an F1 hybrid.

Also a method for transferring a mutant myb12 allele or cd allele (e.g. cd2) to another plant is provided, comprising providing a tomato plant comprising a mutant myb12 allele and/or cd allele (e.g. cd2) in its genome, crossing said plant with another tomato plant and obtaining the seeds of said cross. Optionally plants obtained from these seeds may be further selfed and/or crossed and progeny selected comprising the mutant allele(s) and/or selected phenotypically for the presence of the mutant allele(s). E.g. selecting plants producing fruits exhibiting a less colored or a colorless epidermis of the tomato fruit, or pink tomato fruit at the late orange and/or red stages of fruit development will be a selection for the mutant myb12 allele (being in homozygous form). Similarly a mutant cd allele such as the cd2 allele can be transferred and selected for genotypically and/or phenotypically.

As mentioned, it is understood that other mutagenesis and/or selection methods may equally be used to generate mutant plants according to the invention. Seeds may for example be radiated or chemically treated to generate mutant populations. Also direct gene sequencing of myb12 or cd (e.g. cd2) may be used to screen mutagenized plant populations for mutant alleles. For example KeyPoint screening is a sequence based method which can be used to identify plants comprising mutant myb12 or cd alleles (Rigola et al. PloS One, March 2009, Vol 4(3):e4761).

Thus, non-transgenic mutant tomato plants which produce lower levels of wild type Myb12 protein in fruits are provided, or which completely lack wild type Myb12 protein in fruits, and which produce loss-of-function myb12 protein or reduced-function myb12 protein in fruits due to one or more mutations in one or more endogenous myb12 alleles, are provided. These mutants may be generated by mutagenesis methods, such as TILLING or variants thereof, or by any other method. Myb12 alleles encoding loss-of-function Myb12 protein or reduced-functional Myb12 protein may be isolated and sequenced or may be transferred to other plants by traditional breeding methods.

Likewise, non-transgenic mutant tomato plants which produce lower levels of wild type CD protein (e.g. CD2 protein) in fruits are provided, or which completely lack wild type CD protein (e.g. CD2 protein) in fruits, and which produce loss-of-function cd protein (e.g. cd2 protein) or reduced-function cd protein (e.g. cd2 protein) in fruits due to one or more mutations in one or more endogenous cd (e.g. cd2) allele, are provided. These mutants may be generated by mutagenesis methods, such as TILLING or variants thereof, or by any other method. CD alleles encoding loss-of-function CD protein (e.g. CD1, CD2 or CD3) or reduced-functional CD protein (e.g. CD1, CD2 or CD3) may be isolated and sequenced or may be transferred to other plants by traditional breeding methods.

Especially non-transgenic mutant tomato plants which produce lower levels of wild type Myb12 protein in fruits are provided, or which completely lack wild type Myb12 protein in fruits, and which produce loss-of-function myb12 protein or reduced-function myb12 protein in fruits due to one or more mutations in one or more endogenous myb12 alleles, and which additionally produce lower levels of wild type CD protein (e.g. CD2 protein) in fruits are provided, or which completely lack wild type CD protein (e.g. CD2 protein) in fruits, and which produce loss-of-function cd protein (e.g. cd2 protein) or reduced-function cd protein (e.g. cd2 protein) in fruits due to one or more mutations in one or more endogenous cd (e.g. cd2) allele, are provided.

Any part of the plant, or of the progeny thereof, is provided, including harvested fruit, harvested tissues or organs, seeds, pollen, flowers, ovaries, etc. comprising a mutant myb12 allele and/or mutant cd allele according to the invention in the genome. Also plant cell cultures or plant tissue cultures comprising in their genome a mutant myb12 allele and/or a mutant cd allele are provided. Preferably, the plant cell cultures or plant tissue cultures can be regenerated into whole plants comprising a mutant myb12 allele and/or mutant cd allele in its genome. Also double haploid plants (and seeds from which double haploid plants can be grown), generated by chromosome doubling of haploid cells comprising an myb12 mutant allele and/or cd mutant allele, and hybrid plants (and seeds from which hybrid plants can be grown) comprising a mutant myb12 and/or cd allele in their genome are encompassed herein, whereby in one aspect the double haploid plants and hybrid plants comprising the mutant myb12 allele exhibit a less colored or colorless epidermis, of the tomato fruit at the late orange and/or red stages of fruit development when compared to Solanum lycopersicum being homozygous for the wild type Myb12 allele, and/or whereby in one aspect the double haploid plants and hybrid plants comprising the mutant cd allele exhibit significantly glossier tomato fruit at the red stages of fruit development when compared to Solanum lycopersicum being homozygous for the wild type CD allele.

A plant part can be propagating or non-propagating, for example a non-propagating plant cell in particular a non-propagating plant cell comprising in its genome the mutant myb12 allele of the invention as disclosed herein is provided. In one embodiment the invention relates to a non-propagating plant cell comprising a the mutant myb12 allele of the invention as disclosed herein and comprising a mutation in an allele involved in cuticle development as disclosed herein. In a further embodiment, the invention relates to a non-propagating plant cell comprising a the mutant myb12 allele of the invention as disclosed herein and comprising a mutant cd2 allele of the invention.

The invention further relates to an endogenous myb12 protein having at least one human-induced non-transgenic mutation selected from G50R of SEQ ID NO: 1 or wherein said mutant myb12 protein comprises a deletion of the amino acids 61 to 338 of SEQ ID NO: 1, or in variants thereof, said variants having at least 85% amino acid sequence identity to SEQ ID NO: 1; or an endogenous myb12 allele encoding such protein.

Preferably, the mutant plants also have good other agronomic characteristics, i.e. they do not have reduced fruit numbers and/or reduced fruit quality compared to wild type plants. In a preferred embodiment the plant is a tomato plant and the fruit is a tomato fruit, such as a processing tomato, fresh market tomato of any shape or size or flesh color. Thus, also harvested products of plants or plant parts comprising one or two mutant myb12 alleles and/or one or two mutant cd alleles are provided. This includes downstream processed products, such as tomato paste, ketchup, tomato juice, cut tomato fruit, canned fruit, dried fruit, peeled fruit, etc. The products can be identified by comprising the mutant allele in their genomic DNA.

In one aspect a plant according to the invention (i.e. producing glossy pink fruits) is provided which comprises the genetics for the pink and glossy trait as in a plant deposited under NCIMB 42268.

In another aspect a plant according to the invention (i.e. producing glossy pink fruits) is provided which comprises the genetics for the glossy trait as in a plant deposited under NCIMB 42268 or NCIMB 42269.

In another aspect a plant according to the invention (i.e. producing glossy pink fruits) is provided which comprises the genetics for the pink trait as in a plant deposited under NCIMB 42087 or NCIMB 42088.

In another aspect a plant according to the invention (i.e. producing glossy pink fruits) is provided which comprises the genetics for the pink trait as in a plant deposited under NCIMB 42087 or NCIMB 42088 and comprises the genetics for the glossiness trait as in a plant deposited under NCIMB 42268.

In another aspect a plant according to the invention (i.e. producing glossy pink fruits) is provided which comprises the genetics for the pink trait as in a plant deposited under NCIMB 42087 or NCIMB 42088 and comprises the genetics for the glossiness trait as in a plant deposited under NCIMB 42269.

In still another aspect the invention relates to a plant (or plant parts) of the invention (i.e. producing glossy pink fruits) said plant, or plant parts comprising a cd-allele comprising a mutation resulting in the production of a G736V amino acid substitution in SEQ ID NO: 10 or in variants of SEQ ID NO: 10 having at least 75% amino acid sequence identity to SEQ ID NO: 10 and having the G736V substitution.

In yet another aspect the invention relates to a tomato plant (or plant parts such as fruit, seeds, pollen, cells) of the invention comprising a myb12 allele having one or more mutations resulting in the production of a mutant myb12 protein wherein said mutant myb12 protein comprises a deletion of the amino acids 61 to 338 in SEQ ID NO: 1, or in variants of SEQ ID NO: 1, said variants having at least 95% amino acid sequence identity to amino acids ito 60 SEQ ID NO: 1.

In one aspect the invention relates to a plant (or plant parts) of the invention (i.e. producing glossy pink fruits) said plant, or plant parts comprising a cd-allele comprising a mutation resulting in the production of a G736V amino acid substitution in SEQ ID NO: 10 or in variants of SEQ ID NO: 10 having at least 75% amino acid sequence identity to SEQ ID NO: 10 and having the G736V substitution; and said plant (or part thereof) comprising a myb12 allele having one or more mutations resulting in the production of a mutant myb12 protein wherein said mutant myb12 protein comprises a deletion of the amino acids 61 to 338 in SEQ ID NO: 1, or in variants of SEQ ID NO: 1, said variants having at least 95% amino acid sequence identity to amino acids ito 60 SEQ ID NO: 1.

In one aspect the invention relates to a plant (or plant parts) of the invention (i.e. producing glossy pink fruits) said plant, or plant parts comprising a cd-allele comprising a mutation resulting in the production of a G736V amino acid substitution in SEQ ID NO: 10 and said plant (or part thereof) comprising a myb12 allele having one or more mutations resulting in the production of a mutant myb12 protein wherein said mutant myb12 protein comprises a deletion of the amino acids 61 to 338 in SEQ ID NO: 1.

In one aspect the invention relates to a plant (or plant parts) of the invention (i.e. producing glossy pink fruits) said plant, or plant parts comprising a myb12 allele having one or more mutations resulting in the production of a mutant myb12 protein wherein said mutant myb12 protein comprises a deletion of the amino acids 61 to 338 in SEQ ID NO: 1; and wherein said plant parts further comprise a cd-allele comprising a mutation resulting in the production of a G736V and/or Q708H and/or a D737N amino acid substitution in SEQ ID NO: 10 or in variants of SEQ ID NO: 10 having at least 75% amino acid sequence identity to SEQ ID NO: 10.

The invention further relates to the following embodiments. It is understood that in these embodiments a non-propagating plant cell is a plant cell which is unable to maintain its life by synthesizing carbohydrate and protein from the inorganic substance, such as water, carbon dioxide and mineral salt and so on through photosynthesis.
1. A non-propagating cell of a cultivated plant of the species Solanum lycopersicum said plant being capable of producing pink glossy fruits, comprising a myb12 allele comprising one or more mutations or comprising the y (yellow) gene in homozygous form; and comprising a Cuticle Deficiency (CD) allele comprising one or more mutations in homozygous or heterozygous form, said mutant cd-allele resulting in an increased glossiness of the fruits compared to fruits of plants lacking said mutant cd-allele.
2. The non-propagating plant cell of embodiment 1 wherein the myb12 allele comprising one or more mutations has a mutation selected from the group consisting of mutation in coding region, mutation in non-coding region, mutation in a promotor of the myb12 allele, and in a gene regulating the expression of the myb12 allele.
3. The non-propagating plant cell of embodiment 1 or 2 wherein the myb12 allele comprising one or more mutations results in production of a mutant myb12 protein or lower myb12 protein levels, wherein said lower myb12 protein level is compared with a plant lacking said myb12 allele comprising one or more mutations.
4. The non-propagating plant cell of embodiment 4 wherein said mutant myb12 protein has a Glycine 50 to Arginine (G50R) amino acid substitution in SEQ ID NO: 1; or wherein said mutant myb12 protein consists of SEQ ID NO: 1 and further comprises said G50R amino acid substitution; or wherein said mutant myb12 protein consists of SEQ ID NO: 1 and further comprises said G50R amino acid substitution and up to 8 (e.g. up to 1, 2, 3, or 4) amino acid substitutions or deletions; or wherein said mutant myb12 protein consists of SEQ ID NO: 1 and further comprises said G50R amino acid substitution and up to 8 (e.g. up to 1, 2, 3, or 4) amino acid substitutions or deletions and further consists of an optional sequence of 1-10 (e.g. up to 1, 2, 3, 4, 5, 6, 7, 8, 9 or 10) amino acid residues at the N and/or C terminal of SEQ ID NO: 1; or
   the non-propagating plant cell of embodiment 3 wherein said mutant myb12 protein comprises a deletion of the amino acids 61 to 338 in SEQ ID NO: 1, or in variants thereof, said variants having at least 95% amino acid sequence identity to amino acids 1 to 60 of SEQ ID NO: 1; or
   the plant cell of embodiment 3 wherein the plant comprises the y (yellow) gene.
5. The non-propagating plant cell of anyone of embodiments 1 to 4 wherein the myb12 allele comprising one or more mutations hybridizes under stringent hybridization conditions to SEQ ID NO: 7 and further comprises a guanine (G) to cytosine (C) mutation at nucleotide 1271 (G1271C); or wherein the myb12 allele hybridizes under stringent hybridization conditions to SEQ ID NO: 4 and further comprises a guanine (G) to cytosine (C) mutation at nucleotide 148 (G148C); or
   the non-propagating plant cell of anyone of embodiments 1 to 4 wherein the myb12 allele comprising one or more mutations hybridizes under stringent hybridization conditions to SEQ ID NO: 7 and further comprises a thymine (T) to an adenine (A) mutation at nucleotide position 1305 (T1305); or wherein the myb12 allele hybridizes under stringent hybridization conditions to SEQ ID NO: 4 and further comprises a thymine (T) to an adenine (A) mutation at nucleotide position 182 (T182A).
6. The non-propagating plant cell according to any one of embodiments 1 to 5, comprising the myb12 allele as found in, and which is derivable from or obtainable from (or derived from or obtained from) seed deposited under Accession No. NCIMB 42087 or NCIMB 42088.
7. The non-propagating plant cell according to any one of embodiments 1 to 6, wherein said mutant cd allele is an allele of a gene selected from the group of the CD1 gene, the CD2 gene and the CD3 gene.
8. The non-propagating plant cell according to any one of embodiments 1 to 7 wherein the cd-allele comprising one or more mutations results in production of a mutant cd protein.
9. The non-propagating plant cell according to any one of embodiments 1 to 8 wherein the cd-allele comprising one or more mutations is a cd2 allele encoding a mutant cd2 protein comprising one or more mutations in SEQ ID NO: 10.
10. The non-propagating plant cell according to any one of embodiments 1 to 9, wherein the cutin content and/or cuticle layer thickness is less than 70% of normal cultivated plants of the species Solanum lycopersicum. Preferably, a normal cultivated plants of the species Solanum lycopersicum is a Solanum lycopersicum plant lacking the mutant cd-allele and further comprising the same genetic make-up as the plant cell of the invention.
11. The non-propagating plant cell according to any one of embodiments 1 to 10, wherein the mutant cd-allele is a cd2 allele encoding a protein consisting of SEQ ID NO: 10 and said protein further comprising a G736V amino acid substitution in SEQ ID NO: 10; or wherein the mutant cd-allele is a cd2 allele encoding a functional variant of SEQ ID NO: 10 said variant comprising a G736V amino acid substitution in SEQ ID NO: 10, said variant further comprising up to 16 (e.g. up to 1, 2, 3, 4, 5, 6, 7 or 8) amino acid substitutions or deletions; or wherein the mutant cd-allele is a cd2 allele encoding a functional variant of SEQ ID NO: 10 said variant comprising a G736V amino acid substitution in SEQ ID NO: 10, said variant further comprising up to 16 (e.g. up to 1, 2, 3, 4, 5, 6, 7 or 8) amino acid substitutions or deletions and said variant further consisting of an optional sequence of 1-10 (e.g. 1, 2, 3, 4, 5, 6, 7, 8, 9 or 10) amino acid residues at the N and/or C terminal of SEQ ID NO: 10; or
   wherein the mutant cd-allele is a cd2 allele encoding a protein consisting of SEQ ID NO: 10 and said protein further comprising a Q708H and/or a D737N amino acid substitution in SEQ ID NO: 10; or
   wherein the mutant cd-allele is a cd2 allele encoding a functional variant of SEQ ID NO: 10 said variant comprising a Q708H and/or a D737N amino acid substitution in SEQ ID NO: 10, said variant further comprising up to 16 (e.g. up to 1, 2, 3, 4, 5, 6, 7 or 8) amino acid substitutions or deletions; or
   wherein the mutant cd-allele is a cd2 allele encoding a functional variant of SEQ ID NO: 10 said variant comprising a Q708H and/or a D737N amino acid substitution in SEQ ID NO: 10, said variant further comprising up to 16 (e.g. up to 1, 2, 3, 4, 5, 6, 7 or 8) amino acid substitutions or deletions and said variant further consisting of an optional sequence of 1-10 (e.g. 1, 2, 3, 4, 5, 6, 7, 8, 9 or 10) amino acid residues at the N and/or C terminal of SEQ ID NO: 10.
12. The non-propagating plant cell according to any one of embodiments 1 to 11, wherein the non-propagating plant cell comprises a nucleic acid sequence encoding an mRNA according to SEQ ID NO: 13 or a variant of SEQ ID NO: 13 having at least 90% (e.g. 91, 92, 93, 94, 95, 96, 97, 98, or 99%) nucleic acid sequence identity to SEQ ID NO: 13 and having a thymine at position 2207; or wherein the plant comprises a nucleotide sequence encoding a protein according to SEQ ID NO: 11; or wherein the plant comprises a genomic cd2 sequence having at least 90% (e.g. 91, 92, 93, 94, 95, 96, 97, 98, 99, 99.2%, 99.3%, 99.4%, 99.5%, 99.6%, 99.7% sequence identity with SEQ ID NO: 14 and encoding a mutant CD2 protein comprising one or more of the following amino acid substitutions: G736V, D737N and/or Q708H.
13. The non-propagating plant cell according to any one of embodiments 1 to 12, wherein the mutant cd-allele is a cd2 allele encoding a protein consisting of SEQ ID NO: 10 and said protein further comprising a G736V amino acid substitution in SEQ ID NO: 10; or wherein the mutant cd-allele is a cd2 allele encoding a functional variant of SEQ ID NO: 10 said variant comprising a G736V amino acid substitution in SEQ ID NO: 10, said variant further comprising up to 8 (e.g. up to 1, 2, 3 or 4) amino acid substitutions or deletions; or wherein the mutant cd-allele is a cd2 allele encoding a functional variant of SEQ ID NO: 10 said variant comprising a G736V amino acid substitution in SEQ ID NO: 10, said variant further comprising up to 8 (e.g. up to 1, 2, 3 or 4) amino acid substitutions or deletions and said variant further consisting of an optional sequence of 1-10 (e.g. 1, 2, 3, 4, 5, 6, 7, 8, 9 or 10) amino acid residues at the N and/or C terminal of SEQ ID NO: 10.
14. The non-propagating plant cell according to any one of embodiments 1 to 13, wherein the mutant cd-allele hybridizes under stringent hybridization conditions to SEQ ID NO: 14 and further comprises a guanine (G) to thymine (T) mutation at nucleotide 7171 (G7171T); or wherein the myb12 allele hybridizes under stringent hybridization conditions to SEQ ID NO: 12 and further comprises a guanine (G) to thymine (T) mutation at nucleotide 2207 (T2207G). 15. The non-propagating plant cell according to any one of embodiments 1 to 14, wherein the cd allele comprising one or more mutations is the cd2 allele as present in seeds deposited under NCIMB 42268 or NCIMB 42269.
16. A mutant cd-allele wherein the mutant cd-allele hybridizes under stringent hybridization conditions to SEQ ID NO: 14 and further comprises a guanine (G) to thymine (T) mutation at nucleotide 7171 (G7171T); or wherein the myb12 allele hybridizes under stringent hybridization conditions to SEQ ID NO: 12 and further comprises a guanine (G) to thymine (T) mutation at nucleotide 2207 (T2207G).
17. Use of the mutant cd-allele of embodiment 16 in plant breeding or in the identification of plants.
18. A mutant myb12 allele wherein the mutant myb12 allele hybridizes under stringent hybridization conditions to SEQ ID NO: 7 and further comprises a guanine (G) to cytosine (C) mutation at nucleotide 1271 (G1271C); or wherein the myb12 allele hybridizes under stringent hybridization conditions to SEQ ID NO: 4 and further comprises a guanine (G) to cytosine (C) mutation at nucleotide 148 (G148C); or
   the wherein the myb12 allele hybridizes under stringent hybridization conditions to SEQ ID NO: 7 and further comprises a thymine (T) to an adenine (A) mutation at nucleotide position 1305 (T1305); or wherein the myb12 allele hybridizes under stringent hybridization conditions to SEQ ID NO: 4 and further comprises a thymine (T) to an adenine (A) mutation at nucleotide position 182 (T182A).
19. Use of the mutant myb12-allele of embodiment 18 in plant breeding or in the identification of plants.
20. A mutant myb12 protein consisting of SEQ ID NO: 1 and further comprises a G50R amino acid substitution; or a mutant myb12 protein consists of SEQ ID NO: 1 and further comprising said G50R amino acid substitution and up to 8 (e.g. up to 1, 2, 3, or 4) amino acid substitutions or deletions; or a mutant myb12 protein consisting of SEQ ID NO: 1 and further comprises said G50R amino acid substitution and up to 8 (e.g. up to 1, 2, 3, or 4) amino acid substitutions or deletions and further consisting of an optional sequence of 1-10 (e.g. up to 1, 2, 3, 4, 5, 6, 7, 8, 9 or 10) amino acid residues at the N and/or C terminal of SEQ ID NO: 1; or
   a mutant myb12 protein consisting of amino acids 1 to 60 of SEQ ID NO: 1, or
   a mutant myb12 protein consisting of amino acids 1 to 60 of SEQ ID NO: 1 and further consisting of 1 amino acid substitution or deletion; or
   a mutant myb12 protein consisting of amino acids 1 to 60 of SEQ ID NO: 1 and further consisting of 1 amino acid substitution or deletion and further consisting of an optional sequence of 1-10 (e.g. up to 1, 2, 3, 4, 5, 6, 7, 8, 9 or 10) amino acid residues at the N and/or C terminal of SEQ ID NO: 1.
21. A mutant cd2 protein consisting of SEQ ID NO: 10 and said protein further comprising a G736V amino acid substitution in SEQ ID NO: 10; or a functional variant of SEQ ID NO: 10 said variant comprising a G736V amino acid substitution in SEQ ID NO: 10, said variant further comprising up to 16 (e.g. up to 1, 2, 3, 4, 5, 6, 7 or 8) amino acid substitutions or deletions; or
   a functional variant of SEQ ID NO: 10 said variant comprising a G736V amino acid substitution in SEQ ID NO: 10, said variant further comprising up to 16 (e.g. up to 1, 2, 3, 4, 5, 6, 7 or 8) amino acid substitutions or deletions and said variant further consisting of an optional sequence of 1-10 (e.g. 1, 2, 3, 4, 5, 6, 7, 8, 9 or 10) amino acid residues at the N and/or C terminal of SEQ ID NO: 10; or
   a protein consisting of SEQ ID NO: 10 and said protein further comprising a Q708H and/or a D737N amino acid substitution in SEQ ID NO: 10; or
   a functional variant of SEQ ID NO: 10 said variant comprising a Q708H and/or a D737N amino acid substitution in SEQ ID NO: 10, said variant further comprising up to 16 (e.g. up to 1, 2, 3, 4, 5, 6, 7 or 8) amino acid substitutions or deletions; or
   a functional variant of SEQ ID NO: 10 said variant comprising a Q708H and/or a D737N amino acid substitution in SEQ ID NO: 10, said variant further comprising up to 16 (e.g. up to 1, 2, 3, 4, 5, 6, 7 or 8) amino acid substitutions or deletions and said variant further consisting of an optional sequence of 1-10 (e.g. 1, 2, 3, 4, 5, 6, 7, 8, 9 or 10) amino acid residues at the N and/or C terminal of SEQ ID NO: 10.
22. A plant selection method, said method comprising the step of identifying the mutant allele of embodiment 16 or 18 in said plant material or comprising the step of identifying the mutant protein of embodiment 20 or 21.
23. The non-propagating plant cell according to any one of embodiments 1 to 14 wherein the mutant myb12 allele is the allele as present in seeds deposited under accession number NCIMB 42087 or NCIMB 42088; or
   the non-propagating plant cell according to any one of embodiments 1 to 14 wherein the mutant cd-allele is the allele as present in seeds deposited under accession number NCIMB 42268 or NCIMB 42269; or
   the non-propagating plant cell according to any one of embodiments 1 to 14 wherein the mutant myb12 allele is the allele as present in seeds deposited under accession number NCIMB 42087 or NCIMB 42088 and wherein the mutant cd-allele is the allele as present in seeds deposited under accession number NCIMB 42268; or
   the non-propagating plant cell according to any one of embodiments 1 to 14 wherein the mutant myb12 allele is the allele as present in seeds deposited under accession number NCIMB 42087 or NCIMB 42088 and wherein the mutant cd-allele is the allele as present in seeds deposited under accession number NCIMB 42269.
24. A method of producing a Solanum lycopersicum plant that exhibits pink glossy fruits, comprising the steps of:
   a. providing a recipient Solanum lycopersicum plant or a part thereof;
   b. providing a first donor Solanum lycopersicum plant comprising a myb12 allele comprising one or more mutations or comprising the y (yellow) gene in homozygous form as defined in any of the embodiments or aspect of the invention in this document;
   c. providing a second donor Solanum lycopersicum plant comprising a Cuticle Deficiency (CD) allele comprising one or more mutations in homozygous or heterozygous form, said mutant cd-allele resulting in an increased glossiness of the fruits compared to fruits of plants lacking said mutant cd-allele; as defined in any of the embodiments or aspect of the invention in this document;
   d. crossing the recipient plant and the first donor plant;
   e. selecting progeny plants that exhibit pink fruits;
   f. crossing the progeny plants of step e with the second donor plant;
   g. selecting progeny plants that exhibit pink glossy fruits and comprise the Cuticle Deficiency (CD) allele comprising one or more mutations.
25. A method of producing a Solanum lycopersicum plant that exhibits pink glossy fruits, comprising the steps of:
   a. providing a recipient Solanum lycopersicum plant or a part thereof;
   b. providing a first donor Solanum lycopersicum plant comprising a myb12 allele comprising one or more mutations or comprising the y (yellow) gene in homozygous form as defined in any of the embodiments or aspect of the invention in this document;
   c. providing a second donor Solanum lycopersicum plant comprising a Cuticle Deficiency (CD) allele comprising one or more mutations in homozygous or heterozygous form, said mutant cd-allele resulting in an increased glossiness of the fruits compared to fruits of plants lacking said mutant cd-allele; as defined in any of the embodiments or aspect of the invention in this document;
   d. crossing the recipient plant and the second donor plant;
   e. selecting progeny plants that exhibit glossy fruits and comprise Cuticle Deficiency (CD) allele comprising one or more mutations;
   f. crossing the progeny plants of step e with the first donor plant;
   g. selecting progeny plants that exhibit pink glossy fruits.
26. The method of embodiment 24 or 25 wherein the cuticle deficiency (cd) allele comprising one or more mutations is the cd allele as defined in embodiment 13 or 16.
27. The method of embodiment 24 or 25 wherein the myb12 allele comprising one or more mutations is the myb12 allele as defined in embodiment 5.
28. The method of embodiment 24 or 25 wherein the cuticle deficiency (cd) allele comprising one or more mutations is the cd allele as defined in embodiment 13 or 16 and wherein the myb12 allele comprising one or more mutations is the myb12 allele as defined in embodiment 5.
29. The method of embodiment 28 wherein the cuticle deficiency (cd) allele comprising one or more mutations is the cd allele as defined in embodiment 16 and wherein the myb12 allele comprising one or more mutations is the myb12 allele as defined in embodiment 5.

It is understood that whenever reference is made to an allele as present in seeds deposited under a particular accession number this also encompasses an allele as found in, or which is derivable from or obtainable from or derived from or obtained from, or as found in, or which is derivable from or obtainable from or derived from or obtained from in said particular accession number.

### Seed Deposits

A representative sample of seeds of two (2) tomato TILLING mutants (myb12 mutants) according to Example 1, were deposited by Nunhems B. V. and accepted for deposit on 5 Dec. 2012 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32(1)). Seeds were given the following deposit numbers: NCIMB 42087 (mutant 2961) and NCIMB 42088 (mutant 5505).

A representative sample of seeds of two (2) lines comprising a tomato mutant (cd2 mutant) according to Example 3, were deposited by Nunhems B. V. and accepted for deposit on 4 Jul. 2014 at the NCIMB Ltd. (Ferguson Building, Craibstone Estate, Bucksburn Aberdeen, Scotland AB21 9YA, UK) according to the Budapest Treaty, under the Expert Solution (EPC 2000, Rule 32(1)). Seeds were given the following deposit numbers: NCIMB 42268 (mutant 8.17) and NCIMB 42269 (mutant 26428.001), both having the same glossiness mutation (cd2/cd2). Mutant 8.17 also being homozygous for the Myb12 mutant allele as present in mutant 2961, and produces glossy pink fruits. In addition seeds of line 7.9 which is homozygous for both wild type pink (red) alleles and glossy (dull) alleles) were deposited (NCIMB 42267) and accepted for deposit on 4 Jul. 2014 at the NCIMB Ltd.

The Applicant requests that samples of the biological material and any material derived therefrom be only released to a designated Expert in accordance with Rule 32(1) EPC or related legislation of countries or treaties having similar rules and regulation, until the mention of the grant of the patent, or for 20 years from the date of filing if the application is refused, abandoned, withdrawn or deemed to be withdrawn.

Access to the deposit will be available during the pendency of this application to persons determined by the Director of the U.S. Patent Office to be entitled thereto upon request. Subject to 37 C.F.R. §1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

### EXAMPLES

### General Methods

PCR amplification products were directly sequenced by a service company (BaseClear, The Netherlands, http://www.baseclear.com/) using the same primers as were used for the amplification. The obtained sequences were aligned using a computer program (CLC Bio Main Work Bench, Denmark, www.clcbio.com) to identify the nucleotide changes.

### Materials

Water used for analyses and mutagenis is tap water filtered in an Milli-Q water Integral system, Milli-Q type Reference A+ supplied with a Q-gard T2 Cartridge and a Quantum TEX Cartridge. Water resistance is >=18 MOhm.

Ethyl Methanesulfonate (EMS) (pure) was obtained from Sigma, product number M0880.

Measurement of Tomato Ripening and of Epidermis Color and/or Tomato Fruit Color

Tomato ripening can be measured by various methods known in the art like for example making periodically visual assessments of fruits and/or measurement of fruit firmness or softening, measurement of lycopene contents in the tomato fruits, ethylene production by the fruits, color of the fruits or any alternative method or combination of methods. Fruit firmness can for example be measured by evaluating resistance to deformation in units of for example 0.1 mm as measured with a penetrometer fitted with a suitable probe (e.g. a probe of 3 mm) (Mutschler et al, 1992, Hortscience 27 pp 352-355) (Martinez et al 1995 Acta Horticulturae 412 pp 463-469). Alternative methods exist in the art, such as use of a texturometer (Bui et al. 2010; International Journal of Food Properties, Volume 13, Issue 4 pp 830 846).

Fruit color can be classified by the U.S. standards for grades of fresh tomato (U.S. Dept of Agriculture, 1973, US standards for grades of fresh tomatoes, U.S. Dept Agr. Agr. Mktg. Serv., Washington D.C.), measuring the color with a chromometer (Mutschler et al, 1992, Hortscience 27 pp 352-355) or by comparing the color to a color chart like the Royal Horticultural Society (RHS) Color Chart (www.rhs.org.uk).

Alternatively, external color of tomato fruit can be measured by a chromometer resulting in three parameters: lightness, and chromaticity coordinates on a green to red scale and on a blue to yellow scale (Liu et al, 2003, Plant Biotechnology Journal 1, pp 195-207).

Lycopene content can be determined according to the reduced volumes of organic solvents method of Fish et al. A quantitative assay for lycopene that utilizes reduced volumes of organic solvents. Fish et al. J. Food Compos. Anal. 2002, 15, 309-317. This method can be used to determine lycopene content measured directly on intact tomato fruit while simultaneously estimating the basic physicochemical characteristics: color, firmness, soluble solids, acidity, and pH (Clement et al, J. Agric. Food Chem. 2008, 56, 9813-9818).

Flavonoid content can be determined according to the protocol provided in Ballester et al 2010 (vide supra) or Slimestad et al (Slimestad et al 2008, J. Agric. Food Chem. Vol 56, pp 2436-2441). Or, alternatively, flavonoids can be determined as aglycones or as their glycosides by preparing hydrolyzed and nonhydrolyzed extracts, respectively. Hydrolyzed extracts can be prepared and analyzed by HPLC with photodiode detection (25% acetonitrile in 0.1% trifluoroacetic acid). Dose-response curves of quercetin, naringenin, and kaempferol (0 to 20_g/mL) can be established to quantify these compounds in the hydrolyzed extracts. Nonhydrolyzed extracts can be prepared in 75% aqueous methanol with 10 min of sonication. Subsequent HPLC of the flavonoid species extracted can be done with a gradient of 5 to 50% acetonitrile in 0.1% trifluoroacetic acid. Absorbance spectra and retention times of eluting peaks can be compared with those of commercially available flavonoid standards as described in detail by Bovy et al (Bovy et al 2002, The Plant Cell vol 14 pp 2509-2526).

Fruit peel or epidermis was carefully separated from the rest of the tomato fruit (i.e. flesh of tomato fruit) using a scalpel. Color of fruit peel or epidermis was classified visually.

### Example 1

### Mutagenesis

A highly homozygous inbred line used in commercial processing tomato breeding was used for mutagenesis treatment with the following protocol. After seed imbibition on damp Whatman® paper for 24h, 20,000 seeds, divided in 8 batches of 2500 respectively, were soaked in 100 ml of ultrapure water and ethyl methanesulfonate (EMS) at a concentration of 1% in conical flasks. The flasks were gently shaken for 16h at room temperature. Finally, EMS was rinsed out under flowing water. Following EMS treatment, seeds were directly sown in the greenhouse. Out of the 60% of the seeds that germinated, 10600 plantlets were transplanted in the field. From these 10600 plantlets, 1790 were either sterile or died before producing fruit. For each remaining M1 mutant plant one fruits was harvested and its seeds isolated. The obtained population, named M2 population, is composed of 8810 seeds lots each representing one M2 family. Of these, 585 families were excluded from the population due to low seed set.

DNA was extracted from a pool of 10 seeds originating from each M2 seed lot. Per mutant line, 10 seeds were pooled in a Micronic® deepwell tube; http://www.micronic.com from a 96 deep-well plate, 2 stainless balls were added to each tube. The tubes and seeds were frozen in liquid nitrogen for 1 minute and seeds were immediately ground to a fine powder in a Deepwell shaker (Vaskon 96 grinder, Belgium; http://www.vaskon.com) for 2 minutes at 16.8 Hz (80% of the maximum speed). 300 μl Agowa® Lysis buffer P from the AGOWA® Plant DNA Isolation Kit http://www.agowa.de was added to the sample plate and the powder was suspended in solution by shaking 1 minute at 16.8 Hz in the Deepwell shaker. Plates were centrifuged for 10 minutes at 4000 rpm. 75 μl of the supernatant was pipetted out to a 96 Kingfisher plate using a Janus MDT® (Perkin Elmer, USA; http://www.perkinelmer.com) platform (96 head). The following steps were performed using a Perkin Elmer Janus® liquid handler robot and a 96 Kingfisher® (Thermo labsystems, Finland; http://www.thermo.com). The supernatant containing the DNA was diluted with binding buffer (150 μl) and magnetic beads (20 μl). Once DNA was bound to the beads, two successive washing steps were carried out (Wash buffer 1: Agowa wash buffer 1⅓, ethanol ⅓, isopropanol ⅓; Wash buffer 2: 70% ethanol, 30% Agowa wash buffer 2) and finally eluted in elution buffer (100 μl MQ, 0.025 μl Tween).

Grinding ten S. lycopersicum seeds produced enough DNA to saturate the magnetic beads, thus highly homogenous and comparable DNA concentrations of all samples were obtained. Comparing with lambda DNA references, a concentration of 30 ng/l for each sample was estimated. Two times diluted DNA was 4 fold flat pooled. 2 μl pooled DNA was used in multiplex PCRs for mutation detection analysis.

Primers used to amplify gene fragments for HRM were designed using a computer program (Primer3, http://primer3.sourceforge.net/). The length of the amplification product was limited between 200 and 400 base pairs. Quality of the primers was determined by a test PCR reaction that should yield a single product.

Polymerase Chain Reaction (PCR) to amplify gene fragments. 10 ng of genomic DNA was mixed with 4 μl reaction buffer (5×Reaction Buffer), 2 μl 10×LC dye ((LCGreen+dye, Idaho Technology Inc., UT, USA), 5 pmole of forward and reverse primers each, 4 nmole dNTPs (Life Technologies, NY, USA) and 1 unit DNA polymerase (Hot Start II DNA Polymerase) in a total volume of 10 μl. Reaction conditions were: 30s 98° C., then 40 cycles of 10s. 98° C., 15s 60° C., 25s of 72° C. and finally 60s at 72° C.

High Resolution Melt curve analysis (HRM) has been proven to be sensitive and high-throughput methods in human and plant genetics. HRM is a non-enzymatic screening technique. During the PCR amplification dye (LCGreen+ dye, Idaho Technology Inc., UT, USA) molecules intercalate between each annealed base pair of the double stranded DNA molecule. When captured in the molecule, the dye emits fluorescence at 510 nm after excitation at 470 nm. A camera in a fluorescence detector (LightScanner, Idaho Technology Inc., UT, USA) records the fluorescence intensity while the DNA sample is progressively heated. At a temperature dependent on the sequence specific stability of the DNA helices, the double stranded PCR product starts to melt, releasing the dye. The release of dye results in decreased fluorescence that is recorded as a melting curve by the fluorescence detector. Pools containing a mutation form hetero duplexes in the post-PCR fragment mix. These are identified as differential melting temperature curves in comparison to homo duplexes.

The presence of the particular mutation in individual plants was confirmed repeating the HRM analysis on DNA from the individual M2 seed lots of the identified corresponding DNA pool. When the presence of the mutation, based on the HRM profile, was confirmed in one of the four individual M2 family DNA samples, the PCR fragments were sequenced to identify the mutation in the gene.

Once the mutation was known the effect of such an mutation was predicted using a computer program CODDLe (for Choosing codons to Optimize Discovery of Deleterious Lesions, http://www.proweb.org/coddle/) that identifies the region(s) of a user-selected gene and of its coding sequence where the anticipated point mutations are most likely to result in deleterious effects on the gene's function.

Seeds from M2 families that contain mutations with predicted effect on protein activity were sown for phenotypic analysis of the plants.

Homozygous mutants were selected or obtained after selfing and subsequent selection. The effect of the mutation on the corresponding protein and phenotype of the plant was determined.

Seeds containing the different identified mutations were germinated and plants were grown in pots with soil the greenhouse with 16/8 light dark regime and 18° C. night and 22-25° C. day temperature. For each genotype 5 plants were raised. The second, third and fourth inflorescence were used for the analysis. The inflorescences were pruned leaving six flowers per inflorescence that were allowed to set fruit by self-pollination. The dates of fruit set of the first and sixth flower was recorded as was the date of breaker and red stage of the first and sixth fruit. At breaker of the sixth fruit the truss was harvested and stored in an open box in the greenhouse. Condition of the fruits was recorded during the whole ripening period.

At later stages fruit condition was determined based on visual assessment of the fruits and the date when the oldest fruit became ‘bad’ was recorded and further fruit deterioration was recorded (indicated by further fruit softness assessed by pinching the fruits, and visual assessment of dehydration/water loss, breaking of the skin and fungal growth).

The following mutants were identified: mutant 2961, mutant 5505, mutant 5058, mutant 6899, and seeds of the first two mutants were deposited at the NCIMB under the Accession numbers given above. The plants comprising variant Myb12 proteins 5058 and 6899 did not show a colorless peel phenotype and are therefore considered functional variants of Myb12.

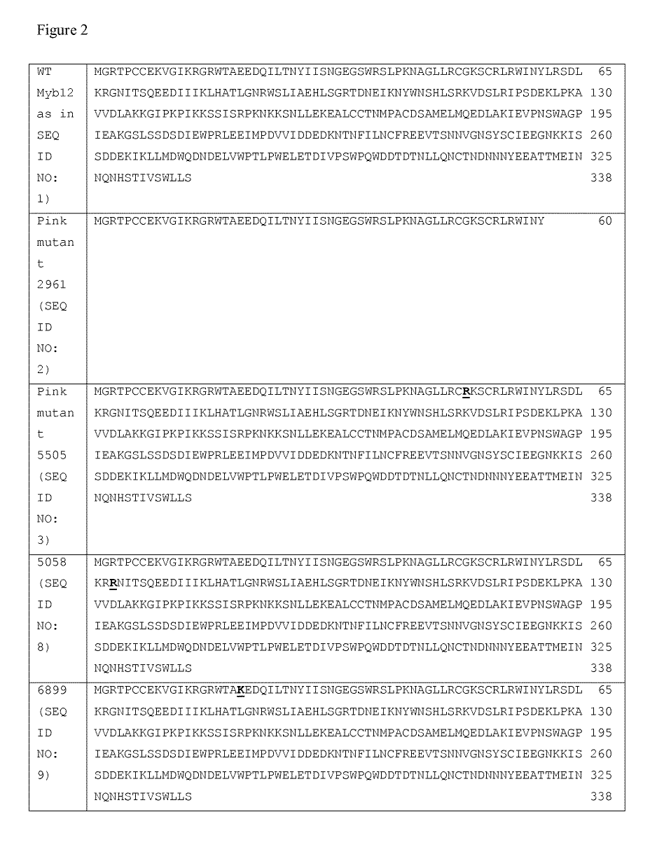

The mutations in the nucleotide sequence compared to the cDNA of wild type Myb12 (as depicted in SEQ ID NO: 4), and its effect on the protein sequence of each mutant has been described above (mutant 2961 and 5505) and is also illustrated in FIG. 2. The protein sequence of mutants 5058 and 6899 is depicted in FIG. 2.

The observed T182A mutation in mutant 2961 and the G148C mutation in mutant 5505 are remarkable in the sense that both mutations are less commonly seen in EMS mutants. EMS normally causes an ethylation of guanine leading to ethylguanine which causes pairing errors of ethylguanine with thymine. This results in G to A and C to T mutations (Krieg (1963) Genetics 48 pp 561-580).

Plants comprising mutations in the target sequence, such as the above mutant plants or plants derived therefrom (e.g. by selfing or crossing) and comprising the mutant myb12 allele, show a normal vegetative growth of all plant parts when compared to wild-type plants except for tomato fruit color of mutant 2961 and 5505. The other two mutants (5058 and 6899) have normal tomato fruit color when compared to wild type. The plants comprising mutations in the target sequence were screened phenotypically for their fruit color.

Plants comprising mutations in the target cd2 sequence, such as the above mutant plants or plants derived therefrom (e.g. by selfing or crossing) and comprising the mutant cd2 allele, show a normal vegetative growth of all plant parts when compared to wild-type plants except for tomato fruit glossiness of mutant 2961 and 5505. The other two mutants (5058 and 6899) have normal tomato fruit color when compared to wild type. The plants comprising mutations in the target sequence were screened phenotypically for their fruit color.

### Example 2

### Fruit Color Determination of Tomato Fruits.

Seeds containing the different mutations were germinated and plants were grown in pots with soil the greenhouse with 16/8 light dark regime and 18° C. night and 22-25° C. day temperature. For each genotype 5 plants were raised. The second, third and fourth inflorescence were used for the analysis. The inflorescences were pruned, leaving six flowers per inflorescence that were allowed to set fruit by self-pollination. The dates of fruit set of the first and sixth flower was recorded as was the date of breaker and red stage of the first and sixth fruit. At red stage of the 4^{th} fruit the truss was harvested and stored in an open box in the greenhouse. Condition of the fruits was recorded during the whole ripening.

Color of the fruit and epidermis was determined visually at the late orange and red stage. Color of fruit and epidermis can for example be characterized by mapping the color to a color code of the color chart of the Royal Horticultural Society (RHS) http://www.rhs.org.uk/Plants/RHS-Publications/RHS-colour-charts.

Fruit of mutant 2961 (mutant 1 in FIG. 2, homozygous) had, at the red-ripe stage, a pink phenotype and a colorless and transparent epidermis. Fruit of mutant 5505 (mutant 2 in FIG. 2, homozygous) also had, at the red-ripe stage, a pink phenotype and a less colored/colorless and transparent epidermis. After some days in red stage, fruits of mutant 5505 (homozygous for the myb12 mutation) developed a small amount of the flavonoid present in the epidermis (i.e. skin) mainly on the shoulders of the fruit. Besides the pink fruit phenotype, both mutants did not have any other apparent pleiotropic effects to the plant.

Fruit of mutant 5505 (mutant 2 heterozygous in FIG. 2, heterozygous), heterozygous for the myb12 mutation (i.e. Myb12/myb12) had, at the red-ripe stage, a red fruit phenotype and an orange-colored epidermis.

Two other mutants were identified (5058 and 6899) in the population of Experiment 1 as described above. Tomato fruits of 5058 were red in the red stage of fruit development. Tomato fruits of mutant 6899 (mutant 3 in FIG. 2, homozygous) also had red fruits. The amino acid substitutions G68R and E20K do, therefore, not seem to affect protein function and these two mutants can be considered as being functional variants of Myb12.

All four (4) myb12 mutants identified comprise a mutation in the myb12 protein as shown in FIG. 2 and the mutated myb12 protein is produced in all of them. However, the effect of the mutation on the function of the myb12 protein differs: only two (2) of them have a pink phenotype which is caused by aberrant myb12 protein function. It thus appears that not all myb12 mutants result in a colorless peel and pink tomato fruit. The pink tomatoes of the invention comprise a particular genetical set-up which resulted in the colorless peel phenotype.

### Example 3

### Fruit Glossiness Determination of Tomato Fruits.

Fruits from various in-house Nunhems' proprietary breeding lines were scored for fruit glossiness (visual scoring, data not shown). One line with high fruit glossiness and having red fruits was selected for crossing with tomato plants capable of producing pink tomatoes. Seeds of this material (mutant 26428.001) were deposited under NCIMB 42269.

Trait inheritance studies revealed that the two traits (pink color and glossiness) were each monogenic, i.e. caused by single genes. Using breeding techniques such as crossing, selfing and backcrossing, initially the two traits could not be combined, suggesting the traits to be located on the same chromosome (chromosome 1) and due to the low recombination frequency, it was assumed that the two alleles for these traits (pink fruit color and glossiness) are located close to each other and might not be recombinable. Eventually the inventors succeeded in obtaining the desired recombination and were able to produce tomato plants homozygous and heterozygous for the mutant myb12 allele and homozygous and heterozygous for the cd2 allele. Seeds of plants being homozygous for the myb12 allele and homozygous for the mutant cd2 allele (mutant 8.17; encoding mutant cd2 protein of SEQ ID NO: 11) were deposited under NCIMB 42268. Plants of the invention being homozygous or heterozygous for any or both of the pink and glossy mutation showed a normal growing behavior and normal plant characteristics (except for fruit color and glossiness), i.e. no negative plant characteristics were present due to the TILLING background of the pink mutant.

Seeds containing the different mutations were germinated and plants were grown in pots with soil the greenhouse with 16/8 light dark regime and 18° C. night and 22-25° C. day temperature. For each genotype 5 plants were raised. The second, third and fourth inflorescence were used for the analysis. The inflorescences were pruned, leaving six flowers per inflorescence that were allowed to set fruit by self-pollination. The dates of fruit set of the first and sixth flower was recorded as was the date of breaker and red stage of the first and sixth fruit. At red stage of the 4^{th} fruit the truss was harvested and stored in an open box in the greenhouse. Condition of the fruits was recorded during the whole ripening.

Fruit glossiness was determined visually at the mature green, orange and red stage (red ripe, or RR). Fruit glossiness was scored on a relative scale ranging from:
+ e.g. for pink fruits (homozygous for mutant pink allele, myb12/myb12), homozygous for wild type Glossy trait (i.e. dull or not glossy due to CD2/CD2); to
++++++ e.g. for red fruits (heterozygous for mutant pink allele, Myb12/myb12, or homozygous for wild type Myb12 allele (Myb12/Myb12), homozygous for mutant type Glossy trait (e.g. cd2/cd2).

Additionally, the degree of glossiness was quantified (at red ripe stage) using a GlossMeter (ETB-0686 Gloss Meter, Graigar, Guangdong China). Using this Gloss Meter specular reflection was measured. The light intensity was registered over a pre-defined reflection angle of 60 degrees. The measurement results of the Gloss Meter were related to the amount of reflected light from a black glass standard (which comes with the ETB-0686 Gloss Meter) with a defined refractive index. The measurement value for this defined standard was equal to 100 μloss units. To prevent the influence of contaminating light (i.e. from the surrounding), the measurements were performed in a dark room. Glossiness of the fruit is not equally distributed all over the fruit. Therefore, glossiness was determined per fruit by measuring light reflection at 4 positions on the pericarp right between the pedicel and blossom end. Per genotype 4 fruits were measured and the average value of these so-obtained 16 measurements was taken as relative value of glossiness. Results of these glossiness measurements are shown in Table 1 below.

Fruits of wild type (7.9) show a normal red tomato colour and reflection at red ripe stage. Fruits of mutant 7.67 (homozygous pink, heterozygous glossy allele from NCIMB 42269 were pink and slightly more glossy than wild type. Fruits of mutant 7.22 (homozygous myb12 allele from NCIMB 42087; homozygous glossy mutant from NCIMB 42269) had pink very glossy fruits.

Conventional breeding with the glossy mutant as deposited under NCIMB 42269 (producing a cd2 protein as in SEQ ID NO: 11) showed that depending on the plant line, plant fruits showed an intermediate phenotype effect on glossiness, i.e. plants heterozygous for the cd2 allele as present in NCIMB 42269 had a glossiness that was higher than wild type plants but lower than fruits of plants homozygous for the mutant cd2 allele.

TABLE 1
Glossiness measurement as determined
using the ETB-0686 Gloss Meter

| | | | Glossiness |
|---|---|---|---|
| | | | [relative to |
| | | | black glass |
| Mutant | Genotype | Phenotype | standard = 100] |
| 7.9 | Wild type for pink | Normal red, | 2.5 |
| (NCIMB | (Myb12/Myb12) and for | normal | |
| 42267) | glossy (CD2/CD2) | glossiness | |
| 7.67 | Homoz myb12 allele from | Pink and | 3.3 |
| | NCIMB 42087 | slightly | |
| | (myb12/myb12); | glossy | |
| | heterozygous glossy mutant | | |
| | from NCIMB 42269 | | |
| | (CD2/cd2) | | |
| 7.22 | Homoz myb12 allele from | Pink and very | 9.9 |
| | NCIMB 42087 | glossy | |
| | (myb12/myb12); | | |
| | homozygous glossy mutant | | |
| | from NCIMB 42269 | | |
| | (cd2/cd2) | | |

1. A cultivated plant of the species Solanum lycopersicum that produces pink glossy fruits, comprising a myb12 allele comprising one or more mutations or comprising they (yellow) gene in homozygous form; and comprising mutant cd allele comprising one or more mutations in homozygous or heterozygous form, said mutant cd allele resulting in an increased glossiness of the fruits compared to fruits of plants lacking said mutant cd allele.

2. The plant of claim 1, wherein the myb12 allele comprising one or more mutations in a coding region, non-coding region, promotor of the myb12 allele, and/or gene regulating the expression of the myb12 allele.

3. The plant of claim 1, wherein the myb12 allele comprising one or more mutations results in production of a mutant myb12 protein or lower myb12 protein levels, wherein said lower myb12 protein level is compared with a plant lacking said myb12 allele comprising one or more mutations.

4. The plant of claim 3, wherein said mutant myb12 protein has a Glycine 50 to Arginine (G50R) amino acid substitution in SEQ ID NO: 1 or in variants thereof, said variants having at least 85% amino acid sequence identity to SEQ ID NO: 1 and having said G50R amino acid substitution; or wherein said mutant myb12 protein comprises a deletion of the amino acids 61 to 338 in SEQ ID NO: 1, or in variants thereof, said variants having at least 95% amino acid sequence identity to amino acids 1 to 60 of SEQ ID NO: 1; or wherein the plant comprises they (yellow) gene.

5. The plant of claim 1, wherein the fruits of said plant comprise a colorless epidermis of the tomato fruit at the late orange and/or red stages of fruit development and wherein the amount of cutin of the fruit cuticle is increased or decreased by at least 15% compared to a plant lacking said mutant cd allele.

6. The plant according to claim 1, wherein the fruits of said plant exhibiting a pink appearance at the late orange and/or red stages of fruit development when said myb12 allele or y gene is in homozygous form.

7. The plant according to claim 1, wherein said mutant cd allele is an allele of the CD1 gene, the CD2 gene or the CD3 gene.

8. The plant according to claim 1, wherein the cd allele comprising one or more mutations results in production of a mutant cd protein.

9. The plant according to claim 1, wherein the cd allele comprising one or more mutations is a cd2 allele encoding a mutant cd2 protein comprising one or more mutations in SEQ ID NO: 10.

10. The plant according to claim 1, wherein the cutin content and/or cuticle layer thickness is less than 70% of normal cultivated plants of the species Solanum lycopersicum.

11. The plant according to claim 1, wherein the cutin content of the tomato fruit is less than 500 μg cm⁻² at the Red Ripe (RR) stage and/or wherein the cuticle layer thickness of the tomato fruit is less than 8 μm, or less than 6 μm at the Red Ripe (RR) stage.

12. The plant according to claim 1, wherein the glossiness level of the fruits at the red ripe (RR) stage is at least twice as high as the glossiness level of fruits of the same line or wild type plants.

13. The plant according to claim 1, wherein the mutant cd allele is a cd2 allele encoding a G736V amino acid substitution in SEQ ID NO: 10 or in a functional variant of SEQ ID NO: 10 said variant having at least 75% amino acid sequence identity to SEQ ID NO: 10; or the mutant cd allele is a cd2 allele encoding a Q708H and/or a D737N amino acid substitution in SEQ ID NO: 10 or in a functional variant of SEQ ID NO: 10 said variant having at least 75% amino acid sequence identity to SEQ ID NO: 10.

14. The plant according to claim 1, wherein the mutant cd allele is a cd2 allele encoding a Glycine to Valine amino acid substitution at position 736 (G736V) of SEQ ID NO: 10 or in a functional variant of SEQ ID NO: 10 said variant having at least 85% amino acid sequence identity to SEQ ID NO: 10 and comprising said G736V amino acid substitution.

15. The plant according to claim 1, wherein the tomato plant comprises a nucleic acid sequence encoding an mRNA according to SEQ ID NO: 13 or a variant of SEQ ID NO: 13 having 70% nucleic acid sequence identity to SEQ ID NO: 13 and having a thymine at position 2207; or wherein the plant comprises a nucleotide sequence encoding a protein according to SEQ ID NO: 11; or wherein the plant comprises a genomic cd2 sequence having at least 70%, 75%, 80%, 85%, 90%, 95%, 98%, 99%, 99.2%, 99.3%, 99.4%, 99.5%, 99.6%, 99.7% sequence identity with SEQ ID NO: 14 and encoding a mutant CD2 protein comprising one or more of the following amino acid substitutions: G736V, D737N and/or Q708H.

16. The plant according to claim 1, wherein the plant is an F1 hybrid plant.

17. Seeds from which a plant according to claim 1 can be grown.

18. Tomato plant parts or progeny of the plant of claim 1 comprising a myb12 allele having one or more mutations, said myb12 allele is:
a) a mutation resulting in production of a mutant myb12 protein, wherein said mutant myb12 protein has a G50R amino acid substitution in SEQ ID NO: 1 or in variants of SEQ ID NO: 1, said variants having at least 85% amino acid sequence identity to SEQ ID NO: 1; and/or
b) a mutation resulting in production of a mutant myb12 protein wherein said mutant myb12 protein comprises a deletion of the amino acids 61 to 338 in SEQ ID NO: 1, or in variants of SEQ ID NO: 1, said variants having at least 85% amino acid sequence identity to SEQ ID NO: 1; and the y (yellow) gene;
and wherein said plant parts further comprise a cd allele comprising a mutation resulting in the production of a G736V and/or Q708H and/or a D737N amino acid substitution in SEQ ID NO: 10 or in variants of SEQ ID NO: 10 having at least 75% amino acid sequence identity to SEQ ID NO: 10.

19. A method for producing a Solanum lycopersicum plant, said method comprising:
(a) crossing a first Solanum lycopersicum plant of claim 1 with a second Solanum lycopersicum plant to obtain seeds; and
(b) growing said seeds;
wherein said Solanum lycopersicum plant grown from the seeds comprises a myb12 allele having one or more mutations wherein said mutations result in production of a mutant myb12 protein, wherein said mutant myb12 protein has a G50R amino acid substitution in SEQ ID NO: 1 or in variants of SEQ ID NO: 1 said variants having at least 85% amino acid sequence identity to SEQ ID NO: 1; or wherein said mutant myb12 protein comprises a deletion of the amino acids 61 to 338 in SEQ ID NO: 1, or in variants of SEQ ID NO: 1, said variants having at least 85% amino acid sequence identity to SEQ ID NO: 1; or wherein the plant comprises they (yellow) gene.

20. The plant according to claim 1, wherein the mutant myb12 allele is the allele as found in, and which is derivable from or obtainable from or derived from or obtained from, or as present in seeds deposited under accession number NCIMB 42087 or NCIMB 42088; or wherein the mutant cd allele is the allele as present in seeds deposited under accession number NCIMB 42268 or NCIMB 42269; or wherein the mutant myb12 allele is the allele as present in seeds deposited under accession number NCIMB 42087 or NCIMB 42088 and wherein the mutant cd allele is the allele as present in seeds deposited under accession number NCIMB 42268; or wherein the mutant myb12 allele is the allele as present in seeds deposited under accession number NCIMB 42087 or NCIMB 42088 and wherein the mutant cd allele is the allele as present in seeds deposited under accession number NCIMB 42269.
